(19) United States
(12) Patent Application Publication
(10) Pub. No.: US 2025/0179137 A1
(43) Pub. Date: Jun. 5, 2025

(54) IL18 RECEPTOR AGONISTS AND METHODS OF USE THEREOF

(71) Applicant: Regeneron Pharmaceuticals, Inc., Tarrytown, NY (US)

(72) Inventors: Jiaxi WU, Pleasantville, NY (US); Aaron CHANG, White Plains, NY (US); Olivia ANTAO, New York, NY (US); Eva-Maria WEICK, New York, NY (US); Amanda DORVILLIERS, New York, NY (US); Erica ULLMAN, Yorktown Heights, NY (US)

(73) Assignee: Regeneron Pharmaceuticals, Inc., Tarrytown, NY (US)

(21) Appl. No.: 18/968,791

(22) Filed: Dec. 4, 2024

Related U.S. Application Data
(60) Provisional application No. 63/606,305, filed on Dec. 5, 2023.

(51) Int. Cl. C07K 14/54; A61P 35/00; C07K 14/715; C07K 16/28

(52) U.S. Cl. CPC C07K 14/54; A61P 35/00; C07K 14/7155; C07K 16/2818; C07K 2317/55

(57) ABSTRACT

The present disclosure relates to IL18 receptor agonists with improved therapeutic profiles.

### 1. CROSS-REFERENCE TO RELATED APPLICATIONS

This application claims the priority benefit of U.S. provisional application No. 63/606,305, filed on Dec. 5, 2023, the contents of which are incorporated herein in their entirety by reference thereto.

### 2. SEQUENCE LISTING

The instant application contains a Sequence Listing which has been submitted electronically and is hereby incorporated by reference in its entirety. Said copy, created on Dec. 4, 2024, is named RGN-038US_SL.xml and is 131,555 bytes in size.

### 3. BACKGROUND

Interleukin 18 (IL-18 or IL18) belongs to the IL-1 superfamily and was first discovered as an interferon gamma (IFN-γ)-inducing factor. This proinflammatory cytokine plays a critical role in inflammation and host defense against infections and is secreted by various types of cells and enhances IFN-γ production in type-1 helper T (Th1) cells and natural killer (NK) cells following activation of NK cell cytotoxicity. IL18 also enhances tumor rejection by its ability to augment the cytotoxic activity of NK and T cells in vivo. The contribution of IL18 signaling to cytokine production and immune response renders IL18 a promising cytokine-treatment due to its broad effect in activating the immune system, such as for the treatment of various cancers.

Despite IL18's potential for novel treatments, clinical development of IL18 has been limited. Unchecked increases in IL18 levels can lead to severe autoinflammatory, neurological, and metabolic reactions. In addition, clinical development of IL18-based therapeutics has been curtailed by a general lack of efficacy, at least in part due to negative regulation of IL18 by a secreted decoy receptor called IL18 binding protein (IL-18BP or IL18BP). IL18BP binds IL18 with extremely high affinity, is upregulated upon IL18 administration, and is prevalent in the tumor microenvironment of human tumors, reducing the available amount of IL18. Furthermore, the inherent instability of IL18 presents challenges in producing, at scale, a molecule having minimal protein aggregation with high expression levels and yield.

Thus, there remains a need for IL18 receptor agonists that will overcome these and other limitations associated with IL18-based treatments.

### 4. SUMMARY

The present disclosure relates to IL18 receptor agonists. In certain aspects, IL18 receptor agonists of the disclosure address the drawbacks of IL18 therapeutic production and efficacy and are characterized by improved expression and/or therapeutic profiles.

The IL18 receptor agonists disclosed herein comprise an IL18 moiety that is masked by an IL18Rα moiety. In certain embodiments, the IL18 receptor agonists comprise, on two polypeptide chains, an IL18 moiety and an IL18Rα moiety. The polypeptide chains of the IL18 receptor agonists are dimerized through a first Fc domain and a second Fc domain to form an Fc region, where both the IL18 moiety and the IL18Rα moiety are N-terminal to the first or second Fc domain. In some embodiments, the IL18 moiety and the IL18Rα moiety are on the same polypeptide chain. In other embodiments, the IL18 moiety and the IL18Rα moiety are on separate polypeptide chains.

The IL18 receptor agonists may further comprise one or more targeting moieties. In such cases, the targeting moiety may be a tumor antigen targeting moiety, as described in Section 6.5. The targeting moiety may be N-terminal or C-terminal to the first or second Fc domain.

In certain aspects, disclosed is an IL18 receptor agonist comprising: a first polypeptide chain comprising, in N- to C-terminal orientation, an IL18 moiety and a first Fc domain; and a second polypeptide chain comprising, in N- to C-terminal orientation, an IL18Rα moiety and a second Fc domain. The IL18 receptor agonist may further comprise a targeting moiety (or a component thereof, e.g., VH of a Fab) C-terminal to the first Fc domain and/or the second Fc domain.

In some aspects, disclosed is an IL18 receptor agonist comprising: a first polypeptide chain comprising, in N- to C-terminal orientation, an IL18 moiety, an IL18Rα moiety, and a first Fc domain; and a second polypeptide chain comprising a second Fc domain. The IL18 receptor agonist may further comprise a targeting moiety (or a component thereof, e.g., VH of a Fab) N-terminal to the second Fc domain.

Exemplary IL18 moieties that can be used in the IL18 receptor agonists of the disclosure are described in Section 6.3.

Exemplary IL18Rα moieties that can be used in the IL18 receptor agonists of the disclosure are described in Section 6.4.

Targeting moieties that can be used in the IL18 receptor agonists of the disclosure are described in Section 6.5 and targeting moiety formats are disclosed in Section 6.5.1.

Fc domains that can be incorporated into the IL18 receptor agonists of the disclosure are described in Section 6.6.

Exemplary IL18 receptor agonists of the disclosure are described in Section 6.2 and numbered embodiments 1 to 157.

The disclosure further provides nucleic acids encoding the IL18 receptor agonists of the disclosure. The nucleic acids encoding the IL18 receptor agonists can be a single nucleic acid (e.g., a vector encoding all polypeptide chains of an IL18 receptor agonist) or a plurality of nucleic acids (e.g., two or more vectors encoding the different polypeptide chains of an IL18 receptor agonist). The disclosure further provides host cells and cell lines engineered to express the nucleic acids and IL18 receptor agonists of the disclosure. The disclosure further provides methods of producing an IL18 receptor agonists of the disclosure. Exemplary nucleic acids, host cells, and cell lines, and methods of producing an IL18 receptor agonists are described in Section 6.8 and specific embodiments 158 to 161.

The disclosure further provides pharmaceutical compositions comprising the IL18 receptor agonist of the disclosure. Exemplary pharmaceutical compositions are described in Section 6.9 and numbered embodiment 165.

Further provided herein are methods of using the IL18 receptor agonist and the pharmaceutical compositions of the disclosure, e.g., for treating cancer, including combination treatment methods. Exemplary methods are described in Section 6.10 and numbered embodiments 162 to 164 and 166 to 180.

### 5. BRIEF DESCRIPTION OF THE FIGURES

FIGS. 1A-1D show the structures of exemplary IL18 receptor agonists. FIG. 1A shows domains and components of exemplary IL18 receptor agonists. FIG. 1B displays an exemplary cis-masked IL18 receptor agonist comprising two polypeptides, each comprising, in the N- to C-terminal configuration [IL18 moiety-IL18Rα moiety-Fc domain]. FIG. 1C displays an exemplary cis-masked IL18 receptor agonist comprising two polypeptides, wherein the first polypeptide comprising in the N- to C-terminal configuration [IL18 moiety-IL18Rα moiety-Fc domain], and the second polypeptide comprising an Fc domain. FIG. 1D displays an exemplary trans-masked IL18 receptor agonist comprising a first polypeptide having the N- to C-terminal configuration [IL18 moiety-Fc domain] and a second polypeptide having the N to C-terminal configuration [IL18Rα moiety-Fc domain]. Triangles in FIGS. 1C-1D represent knob-in-hole mutations that facilitate Fc heterodimerization. Although knob-in-hole mutations are depicted in FIGS. 1C-1D, any other approach that facilitates Fc heterodimerization can be additionally or alternatively incorporated into the IL18 receptor agonists described herein, e.g., as described in Section 6.6.2.

FIGS. 2A-2C show the structures of exemplary targeted IL18 receptor agonists. FIG. 2A shows domains and components of exemplary targeted IL18 receptor agonists. FIG. 2B shows an exemplary cis-masked targeted IL18 receptor agonist comprising a first polypeptide having the N- to C-terminal configuration [IL18 moiety-IL18Rα moiety-Fc domain] and a second polypeptide having the N- to C-terminal configuration [targeting moiety (or component, e.g., VH-CH1 of a Fab)-Fc domain]. FIG. 2C shows an exemplary trans-masked targeted IL18 receptor agonist comprising a first polypeptide having the N- to C-terminal configuration [IL18 moiety-Fc domain-targeting moiety (or component, e.g., VH-CH1 of a Fab)] and a second polypeptide having the N- to C-terminal configuration [IL18Rα moiety-Fc domain-targeting moiety (or component, e.g., VH-CH1 of a Fab)]. Triangles in FIGS. 2B-2C represent optional knob-in-hole mutations that, in some cases, may be used to facilitate Fc heterodimerization. Although the targeting moieties in FIGS. 2B and 2C are illustrated as Fabs, the targeting moieties can be in other formats, e.g., scFvs or other formats described in Section 6.5.

FIG. 3 shows a graph displaying the NFκB activity in EL4/NFκB-Luc cells in response to Fc-fused IL18 constructs.

FIGS. 4A-4B show size exclusion chromatography (SEC) traces of purification of two exemplary IL18 receptor agonists. FIG. 4A shows a representative SEC graph of purification of a trans-masked targeted IL18 receptor agonist. FIG. 4B shows a representative SEC graph of purification of a cis-masked targeted IL18 receptor agonist.

FIG. 5 is a graph that shows binding to hPD1 expressed on HEK293T hPD1 cells.

FIGS. 6A-6D show graphs displaying NFκB activity. FIGS. 6A and 6B are graphs that show NFκB activity in EL4/NFκB-Luc/mPD1^{KO} cells, in the absence and presence of mIL18BP, respectively. FIGS. 6C and 6D are graphs that show NFκB activity in EL4/NFκB-Luc/mPD1^{KO} hPD1^{OE} cells, in the absence and presence of mIL18BP, respectively.

FIGS. 7A-7H are graphs showing percent changes in body weight over time. FIG. 7A shows the percent weight change averages for all of the treatment groups. FIG. 7B shows percent weight changes in individual mice that were administered mIL18(CS2)×Fc. FIG. 7C shows percent weight changes in individual mice that were administered the trans-masked isotype control construct, (mIL18×Rα)-Iso. FIG. 7D shows the percent weight changes in individual mice that were administered the trans-masked PD1-targeted construct, (mIL18×Rα)-PD1. FIG. 7E shows percent weight changes in individual mice that were administered the trans-masked isotype control construct, (mIL18(DR)×Rα)-Iso. FIG. 7F shows the percent weight changes in individual mice that were administered the trans-masked PD1-targeted construct, (mIL18(DR)×Rα)-PD1. FIG. 7G shows percent weight changes in individual mice that were administered the cis-masked isotype control construct, mIL18-Rα×Iso. FIG. 7H shows percent weight changes in individual mice that were administered the cis-masked PD1-targeted construct, mIL18-Rα×PD1.

FIGS. 8A-8C are bar graphs that show cytokine release 1 day after administration of the shown molecules. FIG. 8A is a graph showing IFNγ release, FIG. 8B is a graph showing IL5 release, and FIG. 8C is a graph showing IL6 release.

FIGS. 9A-9F are bar graphs that show blood immune cell profiles 6 days after administration of the shown molecules. FIG. 9A is a graph that shows White Blood Cell (WBC) counts, FIG. 9B is a graph that shows NK cell counts, FIG. 9C is a graph that shows total T cell counts, FIG. 9D is a graph that shows regulatory T cell (Treg) counts, FIG. 9E is a graph that shows CD8⁺ T cell counts, and FIG. 9F is a graph that shows PD1⁺Ki67⁺CD8⁺ T cell counts in the blood samples.

FIGS. 10A-10I are graphs displaying the evaluation of anti-tumor efficacy of IL18 receptor agonists. FIG. 10A shows the averages of tumor volume changes associated with administration of the shown molecules. FIG. 10B shows probability of survival of mice after administration of the shown molecules. FIG. 10C shows percent tumor volume changes in individual mice that were administered mIL18(CS2)×Fc. FIG. 10D shows tumor volume changes in individual mice that were administered the trans-masked non-targeted construct, (mIL18×Rα)-Iso. FIG. 10E shows tumor volume changes in individual mice that were administered the trans-masked PD1-targeted construct, (mIL18×Rα)-PD1. FIG. 10F shows tumor volume changes in individual mice that were administered the trans-masked non-targeted construct, (mIL18(DR)×Rα)-Iso. FIG. 10G shows tumor volume changes in individual mice that were administered the trans-masked PD1-targeted construct, (mIL18(DR)×Rα)-PD1. FIG. 10H shows tumor volume changes in individual mice that were administered the cis-masked non-targeted construct, mIL18-Rα×Iso. FIG. 10I shows tumor volume changes in individual mice that were administered the cis-masked PD1-targeted construct, mIL18-Rα×PD1.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antigen Binding Domain or ABD: The term “antigen binding domain” or “ABD” as used herein refers to the portion of a targeting moiety that is capable of specific, non-covalent, and reversible binding to a target molecule.

Associated: The term “associated” in the context of an IL18 receptor agonist or a component thereof (e.g., an IL18 moiety; an IL18Rα moiety; a targeting moiety such as an antibody or antigen-binding fragment) refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional IL18 receptor agonist. Examples of associations that might be present in an IL18 receptor agonist of the disclosure include (but are not limited to) associations between Fc domains in an Fc region (homodimeric or heterodimeric), associations between VH and VL regions in a Fab or scFv, associations between CH1 and CL in a Fab, and associations between CH3 and CH3 in a domain substituted Fab.

Bivalent: The term “bivalent” as used herein in reference to an IL18 moiety, an IL18Rα moiety, and/or a targeting moiety in an IL18 receptor agonist means an IL18 receptor agonist that has two IL18 moieties, two IL18Rα moieties, and/or two targeting moieties, respectively. Typically, IL18 receptor agonists that are bivalent for an IL18 moiety, IL18Rα moiety, and/or a targeting moiety are dimeric (either homodimeric or heterodimeric).

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, CDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABM definition and the IMGT definition. See, e.g., Kabat, 1991, “Sequences of Proteins of Immunological Interest,” National Institutes of Health, Bethesda, Md. (Kabat numbering scheme); A1-Lazikani et al., 1997, J. Mol. Biol. 273:927-948 (Chothia numbering scheme); Martin et al., 1989, Proc. Natl. Acad. Sci. USA 86:9268-9272 (ABM numbering scheme); and Lefranc et al., 2003, Dev. Comp. Immunol. 27:55-77 (IMGT numbering scheme). Public databases are also available for identifying CDR sequences within an antibody.

EC50: The term “EC50” refers to the half maximal effective concentration of a molecule (such as an IL18 receptor agonist) which induces a response halfway between the baseline and maximum after a specified exposure time. The EC50 essentially represents the concentration of an antibody or IL18 receptor agonist where 50% of its maximal effect is observed. In certain embodiments, the EC50 value equals the concentration of an IL18 receptor agonist that gives half-maximal binding in a fluorescence-assisted cell sorting (FACS) assay as described in Section 8.1.3. In other embodiments, the EC50 value equals the concentration of an IL18 receptor agonist that gives half-maximal relative light unit (RLU) levels in a luciferase activity assay as described in Section 8.1.4.

Epitope: An epitope, or antigenic determinant, is a portion of an antigen (e.g., target molecule) recognized by an antibody or other antigen-binding moiety as described herein. An epitope can be linear or conformational.

Fab: The term “Fab” in the context of a targeting moiety of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey, or human cell line, for example HKB11 cells, PER.C6 cells, HEK cells or CHO cells.

IL18 Receptor Agonist: The term “IL18 receptor agonist” as used herein refers to a molecule comprising an IL18 moiety and which has IL18 activity. The IL18 activity can be greater than, lower than, or about equal to the activity of wild type or recombinant IL18 (e.g., human or murine IL18) in one or more in vitro or in vivo biological assays, for example the luciferase activity assay described in Section 8.1.4.

IL18 Moiety: The term “IL18 moiety” refers to a polypeptide comprising an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a mammalian, e.g., human or murine, wild type IL18 amino acid sequence.

IL18Rα Moiety: The term “IL18Rα moiety” refers to a polypeptide comprising an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL18 binding portion of a mammalian, e.g., human or murine, IL18 receptor subunit alpha (IL18Rα). In some embodiments, the IL18 binding portion of IL18Rα comprises or consists of the extracellular domain of the receptor subunit. In some embodiments, the IL18 binding portion of IL18Rα is domains D1-D3 of human IL18Rα. In some embodiments, the IL18 binding portion of IL18Rα is domains D2-D3 of human IL18Rα. In some embodiments, the IL18 binding portion of IL18Rα is domain D3 of human IL18Rα.

Monovalent: The term “monovalent” as used herein in reference to an IL18 moiety, IL18Rα moiety, and/or a targeting moiety in an IL18 receptor agonist means an IL18 receptor agonist that has only a single IL18 moiety, IL18Rα moiety, and/or targeting moiety, respectively.

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Single Chain Fv or scFv: The term “single chain Fv” or “scFv” as used herein refers to a polypeptide chain comprising the VH and VL domains of antibody, where these domains are present in a single polypeptide chain.

Specifically (or selectively) binds: The term “specifically (or selectively) binds” as used herein means that a targeting moiety, e.g., an antibody, or antigen binding domain (“ABD”) thereof, forms a complex with a target molecule that is relatively stable under physiologic conditions. Specific binding can be characterized by a K_{D} of about 5×10⁻²M or less (e.g., less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment, e.g., an IL18 receptor agonist or a component targeting moiety, to a target molecule are well known in the art and include, for example, equilibrium dialysis, surface plasmon resonance (e.g., Biacore assays), fluorescent-activated cell sorting (FACS) binding assays and the like. An IL18 receptor agonist of the disclosure comprising a targeting moiety or an ABD thereof that specifically binds a target molecule from one species can, however, have cross-reactivity to the target molecule from one or more other species.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Target Molecule: The term “target molecule” as used herein refers to any biological molecule (e.g., protein, carbohydrate, lipid, or combination thereof) that can be specifically bound by a targeting moiety in an IL18 receptor agonist of the disclosure.

Targeting Moiety: The term “targeting moiety” as used herein refers to any molecule or binding portion thereof (e.g., an immunoglobulin or an antigen binding fragment) that can bind to a biological molecule (e.g., protein, carbohydrate, lipid, or combination thereof) at a site to which an IL18 receptor agonist of the disclosure is to be localized, for example on tumor cells or on lymphocytes in the tumor microenvironment. The targeting moiety can also have a functional activity in addition to localizing an IL18 receptor agonist to a particular site. For example, a targeting moiety that is an anti-PD1 antibody or an antigen binding portion thereof can also exhibit anti-tumor activity or enhance the anti-tumor activity by an IL18 mutein by inhibiting PD1 signaling.

Treat, Treatment, Treating: As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more IL18 receptor agonists of the disclosure. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Universal Light Chain: The term “universal light chain” as used herein in the context of a targeting moiety refers to a light chain polypeptide capable of pairing with the heavy chain region of the targeting moiety and also capable of pairing with other heavy chain regions. Universal light chains are also known as “common light chains.”

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 6.2. IL18 Receptor Agonists

The present disclosure provides IL18 receptor agonists comprising an IL18 moiety and an IL18Rα moiety, arranged so that the IL18Rα moiety diminishes (“masks”) the activity of the IL18 moiety. Without wishing to be bound by theory, the inclusion of an IL18Rα moiety capable of masking the IL18 moiety is understood to result in a molecule having reduced systemic toxicity when administered to a subject relative to IL18-containing molecules lacking such a masking component. Typically, the IL18 receptor agonists are dimeric and comprise two Fc domains that associate to form an Fc region, N-terminal to which are the IL18 moiety and the IL18Rα moiety.

In various embodiments, the IL18 receptor agonists are (a) monovalent or bivalent for the IL18 moiety, (b) monovalent or bivalent for the IL18Rα moiety, and/or (b) monovalent or bivalent for a targeting moiety (if present).

Accordingly, the IL18 receptor agonists of the disclosure generally comprise: (a) a first Fc domain and a second Fc domain capable of associating to form an Fc region; (b) an IL18 moiety; and (c) an IL18Rα moiety. In some embodiments, the IL18 moiety is on the same polypeptide as the IL18Rα moiety, while in other embodiments the IL18 moiety and IL18Rα moiety are on different polypeptides. Examples of suitable IL18 moieties are described in Section 6.3 and examples of suitable IL18Rα moieties are described in Section 6.4.

The IL18 receptor agonist can include one or more linker sequences connecting the various components of the molecule, for example connecting any two of an IL18 moiety, an IL18Rα moiety, an Fc domain, and a targeting moiety (or component thereof). Exemplary linkers are described in Section 6.7.

Below are some illustrative configurations of IL18 receptor agonists of the present disclosure, in an N- to C-terminal orientation:

(1) Configuration 1: A homodimer comprising two polypeptides, each comprising: IL18 moiety-optional linker-IL18Rα moiety-optional linker-Fc domain.

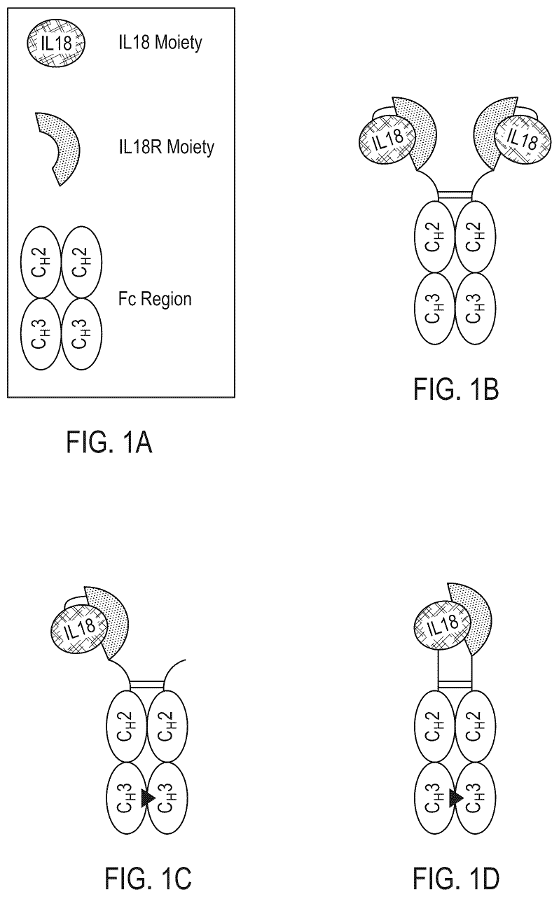

An exemplary Configuration 1 IL18 receptor agonist is depicted in FIG. 1B.

(2) Configuration 2: A heterodimer comprising two polypeptides, A and B:
Polypeptide A: IL18 moiety-optional linker-IL18Rα moiety-optional linker-Fc domain; and
Polypeptide B: Fc domain.

An exemplary Configuration 2 IL18 receptor agonist is depicted in FIG. 1C.

(3) Configuration 3: A heterodimer comprising two polypeptides, A and B:
Polypeptide A: IL18 moiety-optional linker-Fc domain; and
Polypeptide B: IL18Rα moiety-optional linker-Fc domain.

An exemplary Configuration 3 IL18 receptor agonist is depicted in FIG. 1D.

(4) Configuration 4: A heterodimer comprising two polypeptides, A and B:
Polypeptide A: IL18 moiety-IL18Rα moiety-Fc domain; and
Polypeptide B: Targeting moiety-optional linker-Fc domain.

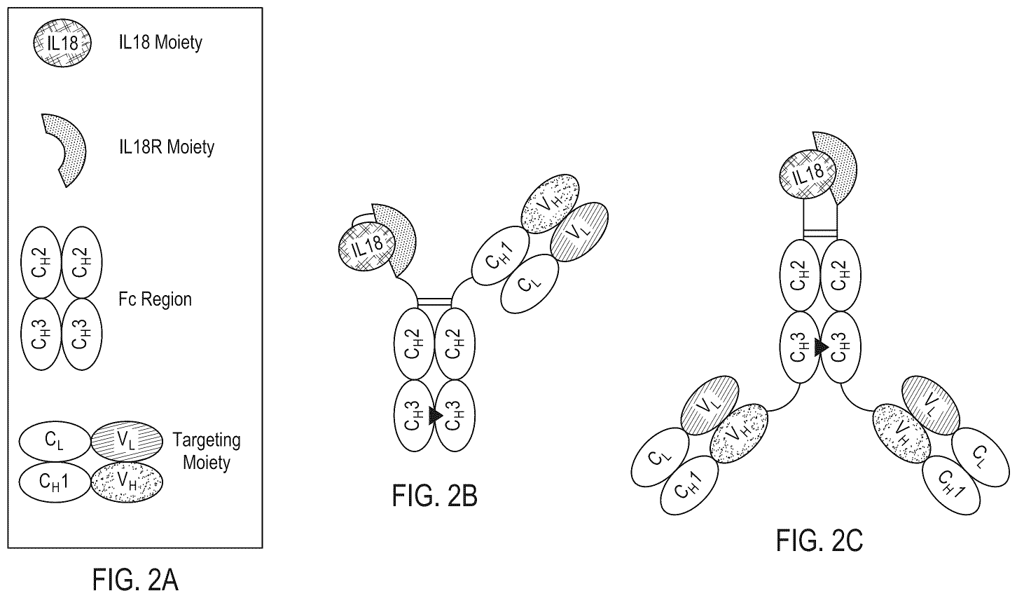

An exemplary Configuration 4 IL18 receptor agonist is depicted in FIG. 2B.

(5) Configuration 5: A heterodimer comprising two polypeptides, A and B:
Polypeptide A: IL18 moiety-optional linker-Fc domain-optional linker-targeting moiety; and
Polypeptide B: IL18Rα moiety-optional linker-Fc domain-optional linker-targeting moiety.

An exemplary Configuration 5 IL18 receptor agonist is depicted in FIG. 2C.

In the IL18 receptor agonists of the disclosure, when the targeting moiety is a Fab, the Fab can be composed of two polypeptide chains, one polypeptide chain bearing the heavy chain variable region and the other polypeptide chain bearing the light chain variable region. Alternatively, an scFv can be used as a targeting moiety, in which the heavy and light chain variable regions of the targeting moiety are fused to one another in a single polypeptide. Additional targeting moiety formats are also contemplated herein and include those described in section 6.5.1.

The IL18 receptor agonists of the disclosure and/or the IL18 moieties in the IL18 receptor agonists of the disclosure can have amino acid modifications that result in a reduction of binding affinity to an IL18 binding protein (IL18BP) as compared to wild type IL18. Overall, the IL18 receptor agonists of the disclosure and/or the IL18 moieties in the IL18 receptor agonists of the disclosure can have normal or attenuated binding (i.e., reduced affinity) to IL18BP (e.g., by up to 10-fold, by up to 50-fold, by up to 100-fold, by up to-200 fold, by up to 500-fold, by up to 1,000-fold, by up to 2,000-fold or by up to 5,000-fold). In some embodiments, binding is attenuated by 100- to 5,000-fold, by 200- to 2,000-fold, by 500- to 2,000-fold or by 500- to 1,000-fold. In some embodiments, the IL18BP is human IL18BP (Uniprot identifier O95998).

In certain embodiments, the IL18 receptor agonists and IL18 moieties of the disclosure have one or more amino acid substitutions in in the IL18 amino acid sequence that reduce binding to IL18BP, for example as disclosed in Section 6.3. For example, in some embodiments, an IL18 moiety can have up to 100-fold to 1,000-fold attenuated binding to human IL18BP as compared to wild-type human IL18. Exemplary amino acid substitutions are disclosed in Section 6.3.

Free IL18 has a relatively short serum half-life, and, without being bound by theory, it is believed that the inclusion of an Fc domain improves serum stability and the pharmacokinetic profile of an IL18 receptor agonist. Thus, the Fc domain can be a dual-purpose domain, conferring homodimerization or heterodimerization capability to the IL18 receptor agonist and conferring stabilization properties.

Exemplary targeting moieties are described in Section 6.5 and include an antigen binding domain (e.g., a scFv or Fab) that binds to a tumor antigen.

In some embodiments, the IL18 receptor agonist of the disclosure is composed of two monomers, optionally in association with one or more additional polypeptide chains (e.g., in association with a polypeptide chain comprising the light chain of a Fab targeting moiety). The monomers can be identical, thereby forming a homodimer, or different, thereby forming a heterodimer. The Fc domains that enable multimerization of each monomer of an IL18 receptor agonist can be configured to dimerize together. Exemplary Fc domains are described in Section 6.6.

In some embodiments, an IL18 receptor agonist can include one or more linker sequences connecting the various components of its one or more polypeptide chains, for example (1) an IL18 moiety and an IL18Rα moiety, when present on the same polypeptide chain, (2) an IL18 moiety an Fc domain, (3) an IL18Rα moiety and an Fc domain, (4) an Fc domain and a targeting moiety or component thereof (e.g., an scFv or a heavy chain of a Fab), or (5) any combination of the foregoing. Exemplary linkers are described in Section 6.7.

In certain aspects, an IL18 receptor agonist of the disclosure has a therapeutic index of greater than 1, and preferably greater than 2, and even more preferably greater than 10. In particular embodiments, the therapeutic index is at least about 10, at least about 20, at least about 100, or at least about 200.

Further details of the components of the IL18 receptor agonists of the disclosure are presented below.

### 6.3. The IL18 Moiety

The present disclosure provides IL18 receptor agonists with IL18 moieties and IL18Rα moieties. The IL18 moiety of an IL18 receptor agonist of the disclosure can comprise a wild type or variant IL18 moiety.

Human IL18 lacks a signal peptide and is synthesized in cells as a biologically inactive precursor (proIL18) of 193 amino acids. This precursor is stored in the cytosol until it is processed intracellularly by caspase 1 in the NLRP3 inflammasome into its biologically active mature form. Once released, IL18 is negatively regulated by a decoy receptor called IL18 binding protein (IL18BP), which is a secreted antagonist that binds IL18 with extremely high affinity (KD<1 nM).

In some embodiments, an IL18 moiety comprises or consists of an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL18Ra binding portion of a mammalian, e.g., human or murine IL18. In some embodiments, the mammalian IL18 is full-length human IL18. In other embodiments, the mammalian IL18 is mature human IL18. Human IL18 has the Uniprot identifier Q14116 (uniprot.org/uniprot/Q14116).

Full-length human IL18 has the following amino acid sequence:

(SEQ ID NO: 1)
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMFTVQNED

Mature human IL18 has the following amino acid sequence:

(SEQ ID NO: 2)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED

In some embodiments, an IL18 moiety comprises or consists of an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity to mature human IL18 (SEQ ID NO:2). In some embodiments, the IL18 moiety is mature human IL18 (SEQ ID NO:2).

In some embodiments, the mammalian IL18 is full-length murine IL18, e.g., mouse IL18. In other embodiments, the mammalian IL18 is mature murine IL18, e.g., mouse IL18. The sequence of full-length mouse IL18 has the Uniprot identifier P70380 (uniprot.org/uniprot/P70380).

Full-length mouse IL18 has the following amino acid sequence:

(SEQ ID NO: 3)
MAAMSEDSCVNFKEMMFIDNTLYFIPEENGDLESDNFGRLHCTTAVIRN
INDQVLFVDKRQPVFEDMTDIDQSASEPQTRLIIYMYKDSEVRGLAVTL
SVKDSKMSTLSCKNKIISFEEMDPPENIDDIQSDLIFFQKRVPGHNKME
FESSLYEGHFLACQKEDDAFKLILKKKDENGDKSVMFTLINLHQS

Mature mouse IL18 has the following amino acid sequence:

(SEQ ID NO: 4)
NFGRLHCTTAVIRNINDQVLFVDKRQPVFEDMTDIDQSASEPQTRLIIY
MYKDSEVRGLAVTLSVKDSKMSTLSCKNKIISFEEMDPPENIDDIQSDL
IFFQKRVPGHNKMEFESSLYEGHFLACQKEDDAFKLILKKKDENGDKSV
MFTLINLHQS

In certain embodiments, the IL18 moiety comprises one or more amino acid substitutions that reduce binding to IL18BP. For example, in some embodiments, the IL18 moiety can have up to 1,000-fold attenuated binding to human IL18BP as compared to wild type human IL18. In some embodiments, the IL18 moiety can have up to 100-fold, up to 50-fold, up to 25-fold, up to 20-fold, up to 15-fold, up to 10-fold, or up to 5-fold attenuated binding to human IL18BP as compared to wild type human IL18.

In some embodiments, the IL18 moiety comprises one amino acid substation. In some other embodiments, the IL18 moiety comprises two or more amino acid substitutions.

Exemplary amino acid substitutions include, but are not limited to, substitutions at Y1, L5, E6, S7, K8, S10, V11, N14, L15, D17, Q18, D23, R27, P28, L29, E31, M33, T34, D35, S36, D37, C38, R39, D40, N41, R44, I46, I49, S50, M51, K53, D54, S55, Q56, P57, G59, M60, A61, V62, T63, S65, K67, C68, E69, I71, C76, E77, I80, I81, N87, P88, D90, K93, T95, K96, S97, Q103, S105, H109, D110, N111, M113, S119, A126, C127, D132, L136, L138, K139, E141, L144, D146, R147, 1149, M150, N155, V153, E156, and D157, as compared to wildtype mature human IL18 (SEQ ID NO:2). An IL18 moiety of an IL18 receptor agonist disclosed herein may comprise any one, two, three, four, five, six, or more substitutions at any of the foregoing positions.

Exemplary amino acid substitutions at residue Y1 of mature human IL18 include Y1F, Y1R, and Y1H.

Exemplary amino acid substitutions at residue L5 of mature human IL18 include L5H, L51, and L5Y.

Exemplary amino acid substitutions at residue E6 of mature human IL18 include E6A and E6Q.

Exemplary amino acid substitutions at residue S7 of mature human IL18 include S7C and S7P.

Exemplary amino acid substitutions at residue K8 of mature human IL18 include K8Q, K8R, K8E, and K8Y.

An exemplary amino acid substitution at residue S10 of mature human IL18 is S10C.

An exemplary amino acid substitution at residue V11 of mature human IL18 is V11I.

Exemplary amino acid substitutions at residue N14 of mature human IL18 include N14C and N14W.

An exemplary amino acid substitution at residue L15 of mature human IL18 is L15C.

An exemplary amino acid substitution at residue D17 of mature human IL18 is D17N.

An exemplary amino acid substitution at residue Q18 of mature human IL18 is Q18L.

Exemplary amino acid substitutions at residue D23 of mature human IL18 include D23S and D23N.

An exemplary amino acid substitution at residue R27 of mature human IL18 is R27Q.

An exemplary amino acid substitution at residue P28 of mature human IL18 is P28C.

An exemplary amino acid substitution at residue L29 of mature human IL18 is L29V.

An exemplary amino acid substitution at residue E31 of mature human IL18 is E31Q.

An exemplary amino acid substitution at residue M33 of mature human IL18 is M33C.

An exemplary amino acid substitution at residue T34 of mature human IL18 is T34P.

Exemplary amino acid substitutions at residue D35 of mature human IL18 include D35E and D35N.

Exemplary amino acid substitutions at residue S36 of mature human IL18 include S36D and S36N.

An exemplary amino acid substitution at residue D37 of mature human IL18 is D37N.

Exemplary amino acid substitutions at residue C38 of mature human IL18 include C38S, C38Q, C38R, C38E, C38L, C38I, C38V, C38K, and C38D.

Exemplary amino acid substitutions at residue R39 of mature human IL18 include R39S and R39T.

An exemplary amino acid substitution at residue D40 of mature human IL18 is D40N.

An exemplary amino acid substitution at residue N41 of mature human IL18 is N41Q.

An exemplary amino acid substitution at residue R44 of mature human IL18 is R44Q.

An exemplary amino acid substitution at residue I46 of mature human IL18 is I46V.

An exemplary amino acid substitution at residue I49 of mature human IL18 is I49C.

Exemplary amino acid substitutions at residue S50 of mature human IL18 include S50C and S50Y.

Exemplary amino acid substitutions at residue M51 of mature human IL18 include M51T, M51K, M51D, M51N, M51E, M51R, M511, M51Q, M51L, M51H, M51F, and M51Y.

Exemplary amino acid substitutions at residue K53 of mature human IL18 include K53A, K53D, K53E, K53G, K53H, K531, K53L, K53M, K53N, K53Q, K53R, K53S, K53T, K53V, K53Y, and K53F.

An exemplary amino acid substitution at residue D54 of mature human IL18 is D54C.

Exemplary amino acid substitutions at residue S55 of mature human IL18 include S55N, S55Q, S55D, S55E, S55T, S55K, and S55R.

Exemplary amino acid substitutions at residue Q56 of mature human IL18 include Q56E, Q56A, Q56R, Q56V, Q56G, Q56K, Q56L, and Q561.

Exemplary amino acid substitutions at residue P57 of mature human IL18 include P57A, P57E, P57T, P57V, P57Q, P57D, P57Y, P57N, P57L, P57G, and P57K.

Exemplary amino acid substitutions at residue G59 of mature human IL18 include G59T and G59A.

Exemplary amino acid substitutions at residue M60 of mature human IL18 include M60I, M60L, M60K, M60Y, M60F, M60Q and M60R.

An exemplary amino acid substitution at residue A61 of mature human IL18 is A61C.

An exemplary amino acid substitution at residue V62 of mature human IL18 is V62C.

An exemplary amino acid substitution at residue T63 of mature human IL18 is T63C.

An exemplary amino acid substitution at residue S65 of mature human IL18 is S65C.

An exemplary amino acid substitution at residue K67 of mature human IL18 is K67Q.

Exemplary amino acid substitutions at residue C68 of mature human IL18 include C68S, C68I, C68F, C68Y, C68D, C68N, C68E, C68Q, and C68K.

An exemplary amino acid substitution at residue E69 of mature human IL18 is E69K.

An exemplary amino acid substitution at residue I71 of mature human IL18 is I71M.

Exemplary amino acid substitutions at residue C68 of mature human IL18 include C76S, C76E, and C76K.

Exemplary amino acid substitutions at residue E77 of mature human IL18 include E77D and E77K.

An exemplary amino acid substitution at residue I80 of mature human IL18 is I80T.

Exemplary amino acid substitutions at residue I81 of mature human IL18 include I81V and I81L.

An exemplary amino acid substitution at residue N87 of mature human IL18 is M87S.

An exemplary amino acid substitution at residue P88 of mature human IL18 is P88C.

An exemplary amino acid substitution at residue D90 of mature human IL18 is D90E.

Exemplary amino acid substitutions at residue K93 of mature human IL18 include K93D and K93N.

An exemplary amino acid substitution at residue T95 of mature human IL18 is T95E.

Exemplary amino acid substitutions at residue K96 of mature human IL18 include K96G and K96Q.

An exemplary amino acid substitution at residue S97 of mature human IL18 is S97N.

Exemplary amino acid substitutions at residue Q103 of mature human IL18 include Q103C, Q103E, Q103I, Q103L, Q103K, Q103P, Q103A, and Q103R.

Exemplary amino acid substitutions at residue S105 of mature human IL18 include S105R, S105D, S105K, S105N, and S105A.

Exemplary amino acid substitutions at residue H109 of mature human IL18 include H109W and H109Y.

Exemplary amino acid substitutions at residue D110 of mature human IL18 include D110N, D110Q, D110R, D110H, D110K, D110E, D110S, and D110G.

Exemplary amino acid substitutions at residue N111 of mature human IL18 include N111H, N111Y, N111D, N111R, N111S, N111G, N111Q, N111T, and N111E.

Exemplary amino acid substitutions at residue M113 of mature human IL18 include M113V, M113R, M113T, M113K, and M1131.

An exemplary amino acid substitution at residue S119 of mature human IL18 is S119L.

An exemplary amino acid substitution at residue A126 of mature human IL18 is A126C.

Exemplary amino acid substitutions at residue C127 of mature human IL18 include C127S, C127W, C127Y, C127F, C127D, C127E, and C127K.

Exemplary amino acid substitutions at residue D132 of mature human IL18 include D132Q and D132E.

An exemplary amino acid substitution at residue L136 of mature human IL18 is L136C.

An exemplary amino acid substitution at residue L138 of mature human IL18 is L138C.

An exemplary amino acid substitution at residue K139 of mature human IL18 is K139C.

Exemplary amino acid substitutions at residue E141 of mature human IL18 include E141K and E141Q.

An exemplary amino acid substitution at residue L144 of mature human IL18 is L144N.

Exemplary amino acid substitutions at residue D146 of mature human IL18 include D146F, D146L, and D146Y.

Exemplary amino acid substitutions at residue D146 of mature human IL18 include R147C and R147K.

An exemplary amino acid substitution at residue I149 of mature human IL18 is I149V.

Exemplary amino acid substitutions at residue M150 of mature human IL18 include M150F and M150T.

Exemplary amino acid substitutions at residue V153 of mature human IL18 include V153I, V153T, and V153A.

Exemplary amino acid substitutions at residue N155 of mature human IL18 include N155C, N155K, and N155H.

An exemplary amino acid substitution at residue E156 of mature human IL18 is E156Q.

Exemplary amino acid substitutions at residue D157 of mature human IL18 include D157A, D157S, and D157N.

An IL18 moiety of an IL18 receptor agonist disclosed herein may comprise any one, two, three, four, five, six, or more of the foregoing amino acid substitutions.

In some embodiments, the IL18 variant includes a 4CS substitution (C38S/C68S/C76S/C127S substitutions) and one or more of additional substitutions including S38C, S38E, S38L, S38Q, S38R, S38V, S38K, S38D, S68C, S68D, S68E, S68F, S68I, S68N, S68Q, S68Y, S68K, S76C, S76E, S76K, S127C, S127D, S127F, S127W, S127K, and S127Y.

The IL18 variants described above, as well as others having reduced binding to IL18BP, are recognized in the art and are described in, for example, US Patent Publication Nos. US 2021/0015891, US 2023/0277625, and US 2023/0146665A1, incorporated herein by reference in their entirety.

Where the IL18 moiety comprises an amino acid sequence having less than 100% identity to mature human IL18 (SEQ ID NO:2), in some embodiments the IL18 moiety comprises no more than one, two, three, four, fixe, or six amino acid substitutions relative to mature human IL18 (SEQ ID NO:2), including any substitution or combination of substitutions described above. In some embodiments, the IL18 moiety comprises or consists of an amino acid sequence having exactly one amino acid substitution relative to mature human IL18 (SEQ ID NO:2). In some embodiments, the IL18 moiety comprises or consists of an amino acid sequence having exactly two amino acid substitutions relative to mature human IL18 (SEQ ID NO:2). In some embodiments, the IL18 moiety comprises or consists of an amino acid sequence having exactly three amino acid substitutions relative to mature human IL18 (SEQ ID NO:2). In some embodiments, the IL18 moiety comprises or consists of an amino acid sequence having exactly four amino acid substitutions relative to mature human IL18 (SEQ ID NO:2). In some embodiments, the IL18 moiety comprises or consists of an amino acid sequence having exactly five amino acid substitutions relative to mature human IL18 (SEQ ID NO:2). In some embodiments, the IL18 moiety comprises or consists of an amino acid sequence having exactly six amino acid substitutions relative to mature human IL18 (SEQ ID NO:2).

### 6.4. The IL18Rα Moiety

IL18 receptor (IL18R) is expressed in immune cells (e.g., T cells and NK cells) and non-immune cells (e.g., neurons and epithelial cells). IL18R is composed of 2 subunits: IL18R subunit alpha (IL18Rα) and IL18R subunit beta (IL18Rβ). The IL18Rα and IL18Rβ subunits are constitutive members of the IL-1R family, and their cytoplasmic domains contain a Toll/IL-1 receptor (TIR) domain, which is shared by the toll-like receptors (TLRs).

IL18 binds to the IL18Rα subunit of IL18R. When triggered by IL18, IL18Rα forms a high affinity heterodimer with IL18Rβ that facilitates downstream signal transduction.

The present disclosure provides IL18 receptor agonists with one or more IL18Rα moieties capable of binding an IL18 moiety. In some aspects, the binding of an IL18Rα moiety to an IL18 moiety attenuates IL18 activity on a target cell.

The extracellular portion of IL18Rα comprises three Ig-like domains, referred to as D1, D2, and D3, wherein D1 is most distal and D3 is most proximal to the plasma membrane. IL18 interacts with IL18Rα by binding the pocket located between D1/D2 and D3 domains.

IL18 receptor agonists of the disclosure include one or more IL18Rα moieties. Each of the one or more IL18Rα moieties is capable of binding an IL18 moiety of the disclosure. An IL18Rα moiety of the disclosure comprises an amino acid sequence having at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL18 binding portion of a mammalian, e.g., human or murine, IL18Rα. In some embodiments, the IL18 binding portion of IL18Rα is the extracellular domain of IL18Rα, or an IL18-binding portion thereof (e.g., the D3 domain of IL18Rα, the D2-D3 domains of IL18Rα, or the D1-D3 domains of IL18Rα).

Human IL18Rα has the Uniprot identifier Q13478. Murine IL18Rα has the Uniprot identifier Q61098.

The sequence of the mature, human IL18Rα extracellular domain (corresponding to amino acids 19-312 of human IL18Rα) is as follows, with the D1 domain in bold, the D2 domain in italics, and the D3 domain underlined:

(SEQ ID NO: 5)
AESCTSRPHITVVEGEPFYLKHCSCSLAHEIETTTKSWYKSSGSQEHVE
LNPRSSSRIALHDCVLEFWPVELNDTGSYFFQMKNYTQKWKLNVIRRNK
HSCFTERQVTSKIVEVKKFFQITCENSYYQTLVNSTSLYKNCKKLLLEN
NKNPTIKKNAEFEDQGYYSCVHFLHHNGKLFNITKTFNITIVEDRSNIV
PVLLGPKLNHVAVELGKNVRLNCSALLNEEDVIYWMFGEENGSDPNIHE
EKEMRIMTPEGKWHASKVLRIENIGESNLNVLYNCTVASTGGTDTKSFI
LVRKADMADIPGHVFTR

The sequences of the D1, D2, and D3 domains of human IL18Ra are:

D1:
(SEQ ID NO: 6)
AESCTSRPHITVVEGEPFYLKHCSCSLAHEIETTTKSWYKSSGSQEHVE
LNPRSSSRIALHDCVLEFWPVELNDTGSYFFQMKNYTQKWKLNVIRR
D2:
(SEQ ID NO: 7)
NKHSCFTERQVTSKIVEVKKFFQITCENSYYQTLVNSTSLYKNCKKLLL
ENNKNPTIKKNAEFEDQGYYSCVHFLHHNGKLFNITKTFNITIVED
D3:
(SEQ ID NO: 8)
RSNIVPVLLGPKLNHVAVELGKNVRLNCSALLNEEDVIYWMFGEENGSD
PNIHEEKEMRIMTPEGKWHASKVLRIENIGESNLNVLYNCTVASTGGTD
TKSFILVRK

In some embodiments, the IL18 binding portion of IL18Rα comprises or consists of the D1, D2, and D3 domains of IL18Rα. In some embodiments, the IL18 binding portion of IL18Rα comprises or consists of the D2 and D3 domains of IL18Rα. In some embodiments, the IL18 binding portion of IL18Rα comprises or consists of the D3 domain of IL18Rα.

In some embodiments, the IL18Rα moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D1 of human IL18Rα (SEQ ID NO:6).

In some embodiments, the IL18Rα moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D2 of human IL18Rα (SEQ ID NO:7), optionally further comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D1 of human IL18Rα (SEQ ID NO:6).

In some embodiments, the IL18Rα moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D3 of human IL18Rα (SEQ ID NO:8), optionally further comprising (a) an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D2 of human IL18Rα (SEQ ID NO:7) and/or (b) an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to D1 of human IL18Rα (SEQ ID NO:6).

The sequence of the region of human IL18Ra containing the D2 and D3 domains is: KKFFQITCENSYYQTLVNSTSLYKNCKKLLLENNKNPTIKKNAEFEDQGYYSCVHFLHHNGKL FNITKTFNITIVEDRSNIVPVLLGPKLNHVAVELGKNVRLNCSALLNEEDVIYWMFGEENGSDP NIHEEKEMRIMTPEGKWHASKVLRIENIGESNLNVLYNCTVASTGGTDTKSFILVRK (SEQ ID NO: 10). In some embodiments, the IL18Rα moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to SEQ ID NO:10.

The sequence of the region of human IL18Ra containing the D1, D2, and D3 domains is: AESCTSRPHITVVEGEPFYLKHCSCSLAHEIETTTKSWYKSSGSQEHVELNPRSSSRIALHDC VLEFWPVELNDTGSYFFQMKNYTQKWKLNVIRRNKHSCFTERQVTSKIVEVKKFFQITCENS YYQTLVNSTSLYKNCKKLLLENNKNPTIKKNAEFEDQGYYSCVHFLHHNGKLFNITKTFNITIV EDRSNIVPVLLGPKLNHVAVELGKNVRLNCSALLNEEDVIYWMFGEENGSDPNIHEEKEMRI MTPEGKWHASKVLRIENIGESNLNVLYNCTVASTGGTDTKSFILVRK (SEQ ID NO:9). In some embodiments, the IL18Rα moiety comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or 100% identity, to SEQ ID NO:9.

The sequence of the mature, murine IL18Rα extracellular domain (corresponding to amino acids 20-312 of murine IL18Rα) is as follows, with the D1 domain in bold, the D2 domain in italics, and the D3 domain underlined:

(SEQ ID NO: 11)
KSCIHRSQIHVVEGEPFYLKPCGISAPVHRNETATMRWFKGSASHEYRE
LNNRSSPRVTFHDHTLEFWPVEMEDEGTYISQVGNDRRNWTLNVTKRNK
HSCFSDKLVTSRDVEVNKSLHITCKNPNYEELIQDTWLYKNCKEISKTP
RILKDAEFGDEGYYSCVFSVHHNGTRYNITKTVNITVIEGRSKVTPAIL
GPKCEKVGVELGKDVELNCSASLNKDDLFYWSIRKEDSSDPNVQEDRKE
TTTWISEGKLHASKILRFQKITENYLNVLYNCTVANEEAIDTKSFVLVR
KEIPDIPGHVFTG

In certain aspects, the IL18Rα moiety can comprise or consist of an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity, or 100% sequence identity, to amino acids 33 to 312 of full-length human IL18Rα (i.e., Uniprot identifier Q13478).

In some embodiments, the IL18Rα moiety comprises or consists of an amino acid sequence having at least about 90%, at least about 95%, at least about 96%, at least about 97%, at least about 98%, at least about 99% or 100% sequence identity to amino acids 33 to 312 of full-length human IL18Rα.

The IL18Rα moiety containing IL18 receptor agonists of the disclosure can have IL18 and IL18Rα moieties on the same polypeptide or on two different polypeptides. In some embodiments, one or more IL18Rα moieties are located N- or C-terminal to the IL18 moiety when located on the same polypeptide. In further embodiments, the IL18Rα moiety-containing IL18 receptor agonists of the disclosure have one or more IL18Rα moieties C-terminal to the IL18 moiety. In some other embodiments, the IL18Rα moiety-containing IL18 receptor agonists of the disclosure have one or more IL18Rα moieties N-terminal to the IL18 moiety.

### 6.5. The Targeting Moiety

Without wishing to be bound by theory, the incorporation of targeting moieties in the IL18 receptor agonists of the disclosure is understood to permit the delivery of high concentrations of IL18 to a desired location, for example the tumor microenvironment, with a concomitant reduction of systemic exposure, resulting in fewer side effects than obtained with wild type IL18. Accordingly, the IL18 receptor agonists of the disclosure may optionally comprise at least one targeting moiety.

In some embodiments, the IL18 receptor agonists are intended to treat cancer, e.g., by inducing a local immune response against tumor tissue. Accordingly, in some embodiments, the targeting moiety binds specifically to a target molecule expressed by a tumor cell or in the tumor cell environment, e.g., an extracellular matrix (“ECM”) antigen, a tumor reactive lymphocyte antigen (e.g., tumor reactive T-cell), a cell surface molecule of tumor or viral lymphocytes, a checkpoint inhibitor, or a tumor-associated antigen (TAA), collectively referred to herein as a “tumor antigen targeting moieties.” The skilled artisan would recognize that the foregoing categories of target molecules are not mutually exclusive and thus a given target molecule may fall into more than one of the foregoing categories of target molecules. For example, some molecules may be considered both TAAs and ECM proteins. Preferably, the ECM antigen, tumor reactive lymphocyte antigen, cell surface molecule of tumor or viral lymphocytes, checkpoint inhibitor, or TAA is a human antigen. The antigen may or may not be present on normal cells.

It is anticipated that any type of tumor and any type of ECM antigen, tumor reactive lymphocyte antigen, cell surface molecule of tumor or viral lymphocytes, checkpoint inhibitor, or TAA may be targeted by the T cell activators of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs and other target molecules associated with the tumor microenvironment are known for virtually any type of cancer.

Non-limiting examples of ECM antigens include syndecan, heparanase, integrins, osteopontin, link, cadherins, laminin, laminin type EGF, lectin, fibronectin, notch, nectin (e.g., nectin-4), tenascin, collagen (e.g., collagen type X) and matrixin.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In certain embodiments, the target molecule is PDL1. In other embodiments, the target molecule is LAG3. In some embodiments, where the target molecule is a checkpoint inhibitor, the tumor antigen targeting moiety is non-blocking or poorly-blocking of ligand-receptor binding. Examples of non-blocking or poorly-blocking anti-PD1 antibodies includes antibodies having VH/VL amino acid sequences of SEQ ID Nos: 2/10 of PCT Pub. No. WO2015/112800A1; SEQ ID Nos: 16/17 of U.S. Pat. No. 11,034,765 B2; SEQ ID Nos. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID Nos 23/24, 3/4 and 11/12 of US Pub. US2022/0056126A1.

In some embodiments, the targeting moieties target the exemplary target molecules set forth in Table A below, together with references to exemplary antibodies or antibody sequences upon which the targeting moiety can be based.

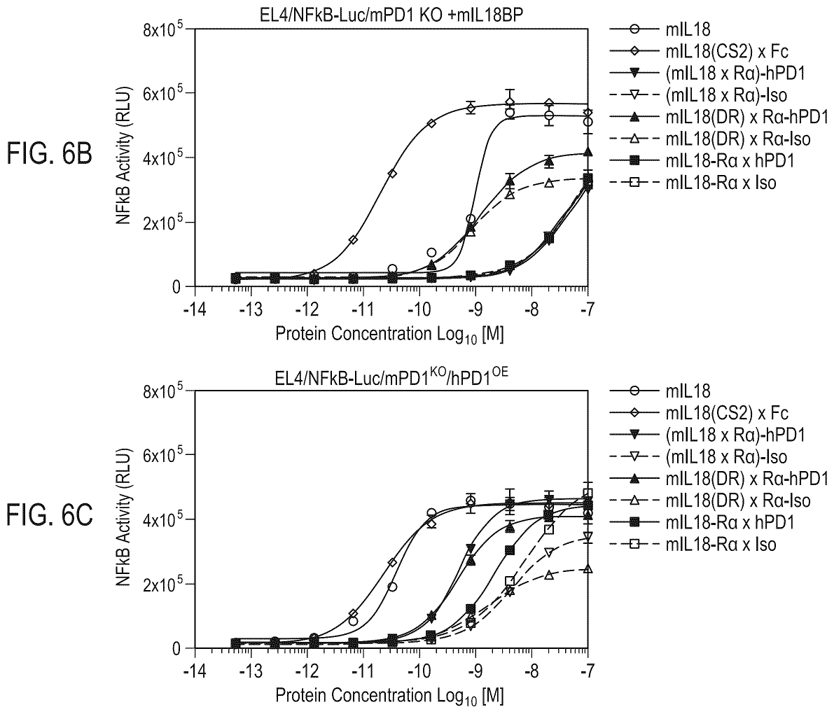

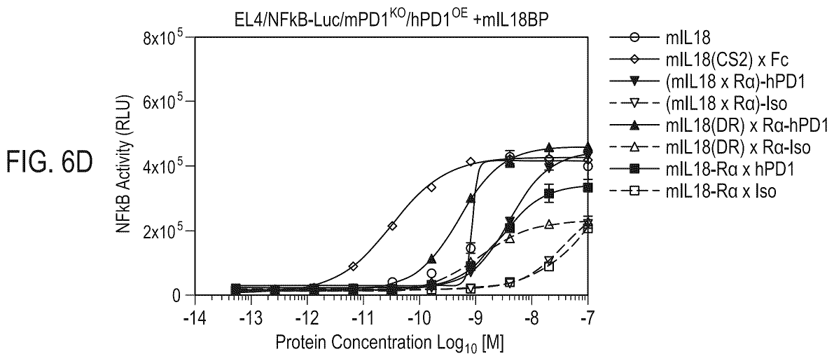

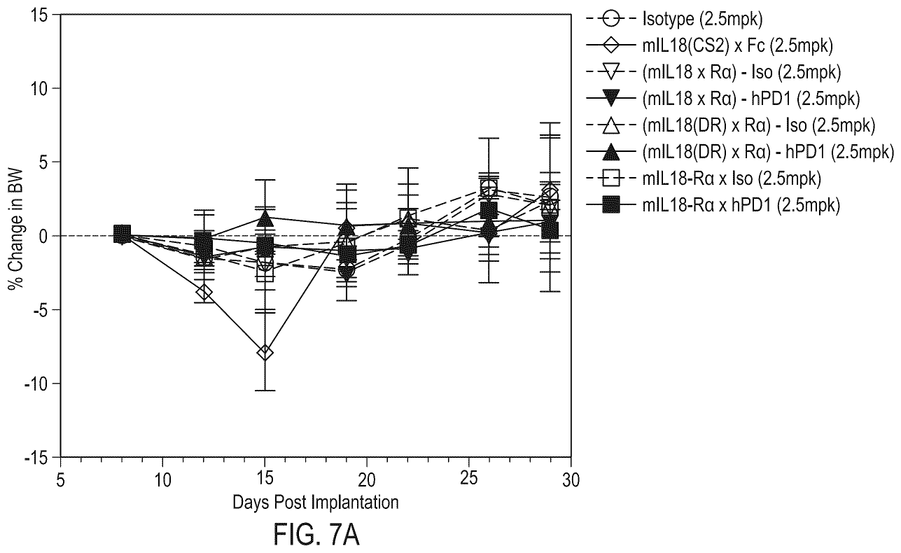

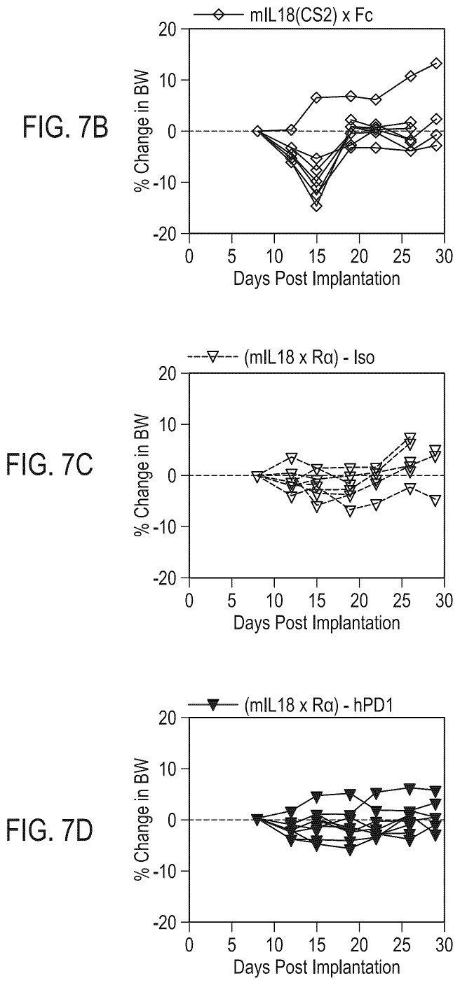

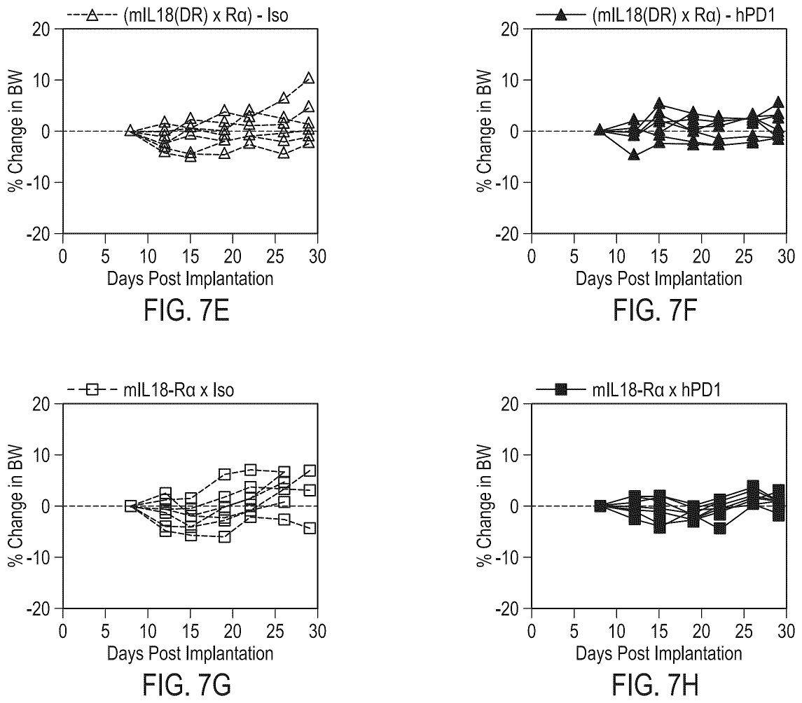

TABLE A
Exemplary Target Molecules

| Target | Antibody Name and/or Binding Sequences |
|---|---|
| 1-92-LFA-3 | Amevive ™ (alefacept) |
| 5T4 | GEN1044 |
| Activin Receptor Type II | Bimagrumab |
| | VH: SEQ ID Nos: 107, 109 of U.S. Pat. No. 8,388,968 B2 |
| | VL: SEQ ID Nos: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID |
| | NO: 21, 26 or 31 of US 2021/0171641 A1. |
| | VL: the VL sequence of the light chain of SEQ ID NO: 20, |
| | 22 or 30 of US 2021/0171641 A1. |
| B7-H3 (CD276) | VH: the VH sequence of the heavy chain of SEQ ID |
| | NO: 21, 29 or 37 of US 2019/0002563 A1. |
| | VL: the VL sequence of the light chain of SEQ ID NO: 17, |
| | 25 or 33 of US 2019/0002563 A1. |
| B7-H3 (CD276) | VH: the VH sequence of the heavy chain of SEQ ID |
| | NO: 146, 147 or 148 of U.S. Pat. No. 10,640,563. |
| | VL: the VL sequence of the light chain of SEQ ID NO: 143, |
| | 144 or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of U.S. Pat. |
| Stimulator | No. 7,138,501 |
| | VL: amino acids 139-249 of SEQ ID NO: 327 of U.S. Pat. |
| | No. 7,138,501. |
| BAFF/B Lymphocyte | VH: amino acids 1-126 of SEQ ID NO: 1321 of U.S. Pat. |
| Stimulator | No. 7,605,236; |
| | VL: amino acids 143-251 of SEQ ID NO: 1049 of U.S. |
| | Pat. No. 7,605,236. |
| BAFF/B Lymphocyte | Belimumab |
| Stimulator | |
| BCMA | VH: the VH sequence of the heavy chain of SEQ ID NO. |
| | 126 of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| Cadherin | The antibodies described in US Pub. No. US |
| | 2006/0039915. |
| N-cadherin | An antibody that binds to the amino acid sequence of |
| | SEQ ID NO: 10, 17 or 18 of US Pub. No. US |
| | 2010/0278821. |
| CD11a | Raptiva ™ (efalizumab) |
| | Sequence in Werther et al., 1996, The Journal of |
| | Immunology 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID |
| | NO: 124 of US Patent Pub. US 2017/0002060 A1 |
| | VL: the VL sequence of the light chain of SEQ ID NO: 125 |
| | of US Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD38 | IB4, HB7 CS/2, clone 90 and NIM-R5 as disclosed in PCT |
| | Pub. WO2015/009726A2 and references cited therein. |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | Flotetuzumab |
| CD206 | Anti-CD206 antibodies having a VH a of SEQ ID NO. 2 |
| | and a VL of SEQ ID NO: 4 of WO2003/040169A2 |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID |
| | Nos: 4-6 of U.S. Pat. No. 8,771,690 B2 |
| | CDR-L1, CDR-L2 and CDR-L3 sequences of SEQ ID |
| | Nos: 1-3 of U.S. Pat. No. 8,771,690 B2 |
| CEA | CEA binding portion of RO6958688/RG7802 from clinical |
| | trial NCT02324257 |
| CEA | Cibisatamab |
| CEA | CEA binding portion of MEDI-565/MT110/AMG211 from |
| | clinical trials NCT01284231 and NCT02291614 |
| | VH: SEQ ID NO: 49 or 51 of PCT Publication No. WO |
| | 2013/012414 A1 |
| | VL: SEQ ID NO: 48 of PCT Publication No. WO |
| | 2013/012414 A1. |
| CEA | Rabetuzumab |
| CEA | Atezolizumab |
| CEA | Cibisatamab |
| CEA | MEDI-565 (AMG211, MT111) |
| CEA | RO6958688 |
| CEA | VH: SEQ ID No. 9 described in WO2022/048883A1 |
| | VL: SEQ ID No. 10 described in WO2022/048883A1 |
| CLDN18.2 | AMG910 |
| Clec9a | Anti-Clec9a antibodies having VH and VL amino acids of |
| | SEQ ID Nos. 43 and 48 of PCT Pub. No. |
| | WO2009/026660A1 |
| Clec9a | Anti-Clec9a antibodies having a VH a of SEQ ID NO. 38 |
| | and a VL of SEQ ID NO: 37 or SEQ ID NO: 43 of PCT Pub. |
| | No. WO2022/073062A1 |
| | Anti-Clec9a antibodies having a VH a of SEQ ID NO. 8 |
| | and a VL of SEQ ID NO: 7 of PCT Pub. No. |
| | WO2022/073062A1 |
| Collagen alpha-4 chain | TRC093 (MT293) |
| Collagen | The collagen binding antibody fragment described in |
| | Liang et al., 2016, Sci. Rep. 5, 18205; doi: |
| | 10.1038/srep18205 (2016). |
| Collagen Type I | Cetuximab (Erbitux) |
| Collagen type X | The amino acid sequences of SEQ ID NO: 1 or 2 of PCT |
| | Pub No. WO 2019/020797. |
| Collagen type X | The amino acid sequences of SEQ ID NO: 1 of PCT Pub |
| | No. WO 2014/180992. |
| Collagen type X | Antibody X34 as described in I. Girkontaite et al., |
| | “Immunolocalization of type X collagen in normal fetal and |
| | adult osteoarthritic cartilage with monoclonal |
| | antibodies,” Matrix Biol 15, 231-238 (1996). |
| Collagen type X | Antibodies X53 or 1H8 or ARC0659 or JF0961 collagen X |
| | polyclonal antibody sold under catalog number PA5- |
| | 115039 or PA5-116871 or PA5-97603 or PA5-49198 from |
| | ThermoFisher Scientific. |
| Collagen type X | Antibody sold under catalog number RDI-COLL10abr from |
| | RDI. |
| Complement C5 | Soliris ™ (eculizumab) |
| | VH: amino acids 1-122 of SEQ ID NO: 10 of U.S. Pat. |
| | No. 6,355,245 |
| | VL: amino acids 3-110 of SEQ ID NO: 9 of U.S. Pat. No. |
| | 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DEC-205 | Anti-DEC-205 antibodies having a VH/VL pair of SEQ ID |
| | NOS. 4/10, 16/22, 28/34, 40/46, 52/58, and 76/82 of PCT |
| | Pub. WO2009/061996A2 |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | Mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID Nos: 80, 84, 88, 92 or 96 |
| | VL: SEQ ID Nos: 82, 86, 90, 94 or 98 |
| EpCAM | G8.8 |
| | HC: SEQ ID NO: 4 of US Patent Pub. No. US |
| | 2020/0317806 A1 |
| | HL: SEQ ID NO: 3 of US Patent Pub. No. US |
| | 2020/0317806 A1 |
| EpCAM | VH: SEQ ID Nos: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| F protein of RSV | Synagic ™ (palivizumab) |
| GD2 | 3F8 |
| Glycoprotein receptor IIb/IIIa | ReoPro ™ (abiciximab) |
| gpA33 | MGD007 |
| GPC3 | ERY974 |
| GUCY2C | PF-07062119 |
| Heparanase | An antibody selected from HP130, HP 239, HP 108.264, |
| | HP 115.140, HP 152.197, HP 110.662, HP 144.141, HP |
| | 108.371, HP 135.108, HP 151.316, HP 117.372, HP |
| | 37/33, HP3/17, HP 201 or HP 102 or an amino acid |
| | sequence of SEQ ID NO: 1-11 described in US Patent |
| | Pub. US 2004/0170631. |
| Her2 | Herceptin ™ (trastuzumab) |
| Her2 | Aldesleukin (proleukine) |
| Her2 | Sargramustim (Leucine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL12/IFN3 | Stelara ™ (ustekinumab) |
| IL1Ra | Antril ™, Kineret ™ (ankinra) |
| IFNR | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| IL12/IFN3 p40 subunit | Stelara ™ (ustekinumab) |
| | VH: SEQ ID NO: 7 of U.S. Pat. No. 6,902,734 |
| | VL: SEQ ID NO: 8 of U.S. Pat. No. 6,902,734 |
| Integrin α4 | Tysabri ™ (natalizumab) |
| | VH: SEQ ID Nos: 11-13 of U.S. Pat. No. 5,840,299 |
| | VL: SEQ ID Nos: 7-8 of U.S. Pat. No. 5,840,299 |
| Integrin α4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrin α5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US |
| | Patent Pub. US 2022/0089744. |
| | VL: SEQ ID NO: 4, 16, 18, 20, 22, 44-57 or 107-116 of US |
| | Patent Pub. US 2022/0089744. |
| KIR | Anti-KIR antibodies having a VH of SEQ ID NO: 5 and a |
| | VL of SEQ ID NO: 3 of PCT Pub. WO2014/066532A1 |
| KIR | Anti-KIR antibodies having a VH of SEQ ID NO: 1 and a |
| | VL of SEQ ID NO: 2 of PCT Pub. WO2012/160448A2 |
| | Anti-KIR antibodies having a VH of SEQ ID NO: 3 and a |
| | VL of SEQ ID NO: 4 of PCT Pub. WO2012/160448A2 |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. 9,902,772. |
| | VL: SEQ ID NO: 2, 3, 4, 5, 6, 7, 9, 11, 13 or 14 of U.S. Pat. |
| | No. 9,902,772. |
| LAG3 | VH: SEQ ID NO: 182 of US Patent Pub. US 2021/0095026. |
| | VL: SEQ ID NO: 88 of US Patent Pub. US 2021/0095026. |
| LAG3 | Antibodies having VH/VL amino acid sequences of SEQ ID |
| | Nos 23/24, 3/4 and 11/12 of US Pub. U.S.2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences |
| | having the amino acid sequences of any one of the |
| | following SEQ ID NO: pairs from US 2018/0118848A1: |
| | 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| NGF | (tanezumab) |
| NKH1A | The monoclonal antibody deposited with ATCC and |
| | assigned accession no. HB8564, as described in U.S. |
| | Pat. No. 4,772,552A |
| NKP46 | Anti-NKP46 antibodies having CDR-H1, CDR-H2 and |
| | CDR-H3 sequences of SEQ ID Nos: 4, 6 and 8 and CDR- |
| | L1, CDR-L2 and CDR-L3 sequences of SEQ ID Nos: 12, |
| | 14 and 16 of PCT Pub. WO2018/047154A1 |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 lumab), a human IgG4 mAb |
| | with the structure described in WHO Drug Information, |
| | Vol. 27, No. 1, pages 68-69 (2013) and whose heavy and |
| | light chain sequences are disclosed in FIG. 7 of US Pub. |
| | No. US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with |
| | the structure described in WHO Drug Information, Vol. 27, |
| | No. 2, pages 161-162 (2013) and whose heavy and light |
| | chain sequences are disclosed in FIG. 6 of US Pub. No. |
| | US20190270812A1 |
| | HC: SEQ ID NO: 21 of US Pub. No. US20190270812A1 |
| | LC: SEQ ID NO: 22 of US Pub. No. US20190270812A1 |
| PD1 | H4H7798N as disclosed in US Pub No. 20150203579 |
| | HC: SEQ ID NO: 330 of US Pub. No. 20150203579 |
| | LC: SEQ ID NO: 331 of US Pub. No. 20150203579 |
| PD1 | H2M7791N disclosed in US Pub No. 20150203579 as |
| | having the following heavy and light chain variable |
| | domains: |
| | VH: SEQ ID NO: 98 of US Pub. No. 20150203579 |
| | VL: SEQ ID NO: 106 of US Pub. No. 20150203579 |
| PD1 | Anti-PD1 antibodies having CDR H1-H3 and CDR L1-L3 |
| | sequences corresponding to the following SEQ ID Nos. of |
| | U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; |
| | and |
| | h) SEQ ID Nos: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID Nos: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 |
| | and 314/186 of PCT Pub. WO2015112800A1. |
| PD1 | Anti-PD1 antibodies disclosed in U.S. Pat. No. |
| | 10,294,299 B2 as having the following SEQ ID NO. pairs |
| | for heavy and light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb whose |
| | heavy and light chain sequences are disclosed as |
| | BAP049-Clone-E in U.S. Pat. No: 9683048 B2. |
| | HC: SEQ ID NO: 91 of U.S. Pat. No: 9,683,048 |
| | LC: SEQ ID NO: 72 of U.S. Pat. No: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. |
| | 7,488,802 as having the following SEQ ID NO. pairs for |
| | heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. |
| | 7,521,051 |
| | as having the following SEQ ID NO. pairs for heavy and |
| | light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. |
| | 8,008,449 |
| | as having the following SEQ ID NO. pairs for heavy and |
| | light chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. |
| | 8,354,509 as having the following SEQ ID NO. pairs for |
| | heavy and light chain full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. |
| | 8,168,757 as having the following SEQ ID NO. pairs for |
| | heavy and light chain variable domains: |
| | SEQ ID Nos. 4/5 |
| | SEQ ID Nos. 12/13 |
| | SEQ ID Nos. 18/19 |
| | SEQ ID Nos. 40/41 |
| | SEQ ID Nos. 47/48 |
| | SEQ ID Nos. 26/27 |
| | SEQ ID Nos. 34/35 |
| | SEQ ID Nos. 55/56 |
| | SEQ ID Nos. 67/68 |
| PD1 | Anti-PD1 antibodies described in PCT Pub. No. |
| | WO2004/004771 |
| PD1 | Anti-PD1 antibodies described in PCT Pub. No. |
| | WO2004/056875 as having the following SEQ ID NO. |
| | pairs for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. |
| | WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT Publication |
| | No: WO 2015/085847 as having the following heavy and |
| | light chain variable domains: |
| | HC: SEQ ID NO: 9 |
| | LC: SEQ ID NO: 10 |
| PD1 | 4A11 described in U.S. Pat. No. 8,008,449 as having |
| | the following heavy and light chain variable domains: |
| | VH: SEQ ID NO: 5 |
| | VL: SEQ ID NO: 12 |
| PD1 | h1H3 Var 6 described in U.S. Pat. No. 9,205,148 as |
| | having the following heavy and light chain variable |
| | domains: |
| | VH: SEQ ID NO: 106 |
| | VL: SEQ ID NO: 98 |
| PD1 | PD1AB-6 described in U.S. Pat. No. 10,428,145 as |
| | having the following heavy and light chain variable |
| | domains: |
| | VH: SEQ ID NO: 13 |
| | VL: SEQ ID NO: 8 |
| PD1 | A1.5 described in U.S. Pat. No. 10,513,558 as having |
| | the following heavy and light chain variable domains: |
| | VH: SEQ ID NO: 21 |
| | VL: SEQ ID NO: 47 |
| PD1 | Anti-PD1 antibodies 244C8-2 and 388D4-3, described in |
| | U.S. Pat. No. 10,544,217 as having the following SEQ |
| | ID NO. pairs for heavy and light chain variable domains, |
| | respectively: |
| | SEQ ID NOs: 85/93 |
| | SEQ ID NOs: 88/96 |
| PD1 | Anti-PD1 antibodies 9B2C6C9 and 76G5B3, described in |
| | PCT Publication No. WO 2017/016497 as having the |
| | following SEQ ID NO. pairs for heavy and light chain |
| | variable domains, respectively: |
| | SEQ ID NOs: 1/5 |
| | SEQ ID NOs: 9/13 |
| PD1 | Anti-PD1 antibodies 1.153.7 hAb and 1.103.11 hAbv2 |
| | described in PCT Publication No. WO 2017/025051 as |
| | having the following SEQ ID NO. pairs for heavy and light |
| | chain variable domains, respectively: |
| | SEQ ID NOs: 61/63 |
| | SEQ ID NOs: 53/55 |
| PD1 | Clone 19 described in U.S. Pat. No. 8,927,697 as |
| | having the following heavy and light chain variable |
| | domains: |
| | VH: SEQ ID NO: 14 |
| | VL: SEQ ID NO: 12 |
| PD1 | PD1-0103-312 described in PCT Publication No. WO |
| | 2017/055443 as having the following heavy and light |
| | chain variable domains: |
| | VH: SEQ ID NO: 57 |
| | VL: SEQ ID NO: 58 |
| PD1 | Anti-PD1 antibodies h4B10-3 and 1353-G08 described in |
| | PCT Publication No. WO 2016/077397 as having the |
| | following SEQ ID NO. pairs for heavy and light chain |
| | variable domains, respectively: |
| | SEQ ID NOs: 264/288 |
| | SEQ ID NOs: 251/275 |
| PD1 | PD-1-27 described in PCT Publication No. WO |
| | 2016/210129 as having the following heavy and light |
| | chain variable domains: |
| | VH: SEQ ID NO: 51 |
| | VL: SEQ ID NO: 49 |
| PD1 | Anti-PD1 antibodies RG1H10 and RG1H10-16C |
| | described in PCT Publication No. WO 2014/194302 as |
| | having the following SEQ ID NO. pairs for heavy and light |
| | chain variable domains, respectively: |
| | SEQ ID NOs: 23/24 |
| | SEQ ID NOs: 40/24 |
| PD1 | HuEH12-VH4-VK3 described in U.S. Pat. No. 9,102,727 |
| | as having the following heavy and light chain |
| | variable domains: |
| | VH: SEQ ID NO: 28 |
| | VL: SEQ ID NO: 32 |
| PD1 | Clone 39 described in U.S. Pat. No. 10,066,013 as |
| | having the following heavy and light chain variable |
| | domains: |
| | VH: SEQ ID NO: 35 |
| | VL: SEQ ID NO: 34 |
| PD1 | tislelizumab |
| PD1 | retifanlimab |
| PD1 | balstilimab |
| PD1 | dostarlimab |
| PD1 | sasanlimab |
| PD1 | sintilimab |
| PD1 | cemiplimab |
| PDL1 | Durvalumab (MEDI4736) |
| | HC: SEQ ID NO: 26 of PCT application No. |
| | WO2020225552 |
| | LC: SEQ ID NO: 27 of PCT application No. |
| | WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. |
| | 7,943,743 as having the following SEQ ID NO. pairs for |
| | heavy and light chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No: 9,624,298 as |
| | having the following heavy and light chain variable |
| | domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID |
| | Nos: 52, 56, and 77 of PCT Pub. No. 2018/080812 |
| | CDR-L1, CDR-L2 and CDR-L3 sequences of SEQ ID Nos: |
| | 65, 42, and 71 of PCT Pub. No. 2018/080812 |
| PDL1 | KN035 (Envafolimab) is a nanobody described as Hu56V2 |
| | in U.S. Pat. No. 11,225,522 as having the VHH SEQ ID |
| | NO: 34 |
| PDL1 | LY3434172 |
| PDL1 | LY3300054 (lodapolimab) described in PCT Pub No: WO |
| | 2017/034916 as having the following heavy and light |
| | chain variable domains: |
| | HC: SEQ ID NO: 10 |
| | L: SEQ ID NO: 11 |
| PDL1 | LDP (lesabelimab, ADG104) described in CN Patent No: |
| | 114225023 as having the following heavy and light chain |
| | variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No: |
| | 10,118,963 as having the following heavy and light chain |
| | variable domains: |
| | HC: SEQ ID NO: 1 |
| | LC: SEQ ID NO: 2 |
| PDL1 | BGB-A333 (garivulimab) described in U.S. Pat. No: |
| | 11,512,132 as having the following heavy and light chain |
| | variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or 145 |
| | of U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences |
| | having the amino acid sequences of any one of the |
| | following SEQ ID NO: pairs from WO 2017/023761A1: |
| | 2/1642; 10/1642; 18/1642; 26/1642; 34/1642; 42/1642; |
| | 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, |
| | PSMA 3.11, PSMA 5.4, PSMA 7.1, PSMA 7.3, PSMA |
| | 10.3, PSMA 1.8.3, PSMA A3.1.3, PSMA A3.3.1, Abgenix |
| | 4.248.2, Abgenix 4.360.3, Abgenix 4.7.1, Abgenix 4.4.1, |
| | Abgenix 4.177.3, Abgenix 4.16.1, Abgenix 4.22.3, |
| | Abgenix 4.28.3, Abgenix 4.40.2, Abgenix 4.48.3, Abgenix |
| | 4.49.1, Abgenix 4.209.3, Abgemx 4.219.3, Abgenix |
| | 4.288.1, Abgenix 4.333.1, Abgemx 4.54.1, Abgenix |
| | 4.153.1, Abgenix 4.232.3, Abgenix 4.292.3, Abgenix |
| | 4.304.1, Abgenix 4.78.1 and Abgenix 4.152.1 described in |
| | WO2003034903A2 |
| | A hybridoma cell line such as: PSMA 3.7 (PTA-3257), |
| | PSMA 3.8, PSMA 3.9 (PTA- 3258), PSMA 3.11 (PTA- |
| | 3269), PSMA 5.4 (PTA-3268), PSMA 7.1 (PTA-3292), |
| | PSMA 7.3 (PTA-3293), PSMA 10.3 (PTA-3247) , PSMA |
| | 1.8.3 (PTA-3906), PSMA A3.1.3 (PTA- 3904), PSMA |
| | A3.3.1 (PTA-3905), Abgenix 4.248.2 (PTA-4427), Abgenix |
| | 4.360.3 (PTA- 4428), Abgenix 4.7.1 (PTA-4429), Abgenix |
| | 4.4.1 (PTA-4556), Abgenix 4.177.3 (PTA-4557), Abgenix |
| | 4.16.1 (PTA-4357), Abgenix 4.22.3 (PTA-4358), Abgenix |
| | 4.28.3 (PTA-4359), Abgenix 4.40.2 (PTA-4360), Abgenix |
| | 4.48.3 (PTA-4361), Abgenix 4.49.1 (PTA-4362), Abgenix |
| | 4.209.3 (PTA-4365), Abgenix 4.219.3 (PTA-4366), |
| | Abgenix 4.288.1 (PTA-4367), Abgenix 4.333.1 (PTA- |
| | 4368), Abgenix 4.54.1 (PTA-4363), Abgenix 4.153.1 |
| | (PTA-4388), Abgenix 4.232.3 (PTA-4389), Abgenix |
| | 4.292.3 (PTA-4390), Abgenix 4.304.1 (PTA-4391), |
| | Abgenix 4.78.1 (PTA-4652), and Abgemx 4.152.1(PTA- |
| | 4653) described in WO 2003/034903A2. |
| | VH of SEQ ID Nos: 2-7 described in WO 2003/034903A2 |
| | VL of SEQ ID Nos: 8-13 described in WO 2003/034903A2 |
| PMSA | VH: SEQ ID Nos: 225, 239, 253, 267, 281, 295, 309, 323, |
| | 337, 351, 365, 379, 393, 407, 421, 435, 449, 463, 477, |
| | 491, 505, 519, 533, 547, 561, 575, 589, 603 or 617 |
| | described in WO 2011/121110A1. |
| | VL SEQ ID Nos: 230, 244, 258, 272, 286, 300, 314, 328, |
| | 342, 356, 370, 384, 398, 412, 426, 440, 454, 468, 482, |
| | 496, 510, 524, 538, 552, 566, 580, 594, 608 or 622 |
| | described in WO 2011/121110A1. |
| | VH and VL SEQ ID Nos: 235, 249, 263, 277, 291, 305, |
| | 319, 333, 347, 361, 375, 389, 403, 417, 431, 445, 459, |
| | 473, 487, 501, 515, 529, 543, 557, 571, 585, 599, 613 or |
| | 627 described in WO 2011/121110A1. |
| PMSA | An anti-PMSA antibody having a VL amino acid sequence |
| | of any one of SEQ ID Nos: 229-312 of US 2022/0119525 |
| | A1 and a VH of SEQ ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of U.S. Patent Pub. 2017/0002060 |
| | VL: SEQ ID NO: 52 of U.S. Patent Pub. 2017/0002060 |
| SLAMF7 | Empliciti ™ (elotuzumab) |
| SSTR2 | XmAb ®18087 |
| STEAP1 | VHCDR1 SEQ ID Nos: 14, 33, 182, 184 or 185 described |
| | in US20210179731A1. |
| | VHCDR2 SEQ ID Nos: 15, 21, 34, 182, 184 or 185 |
| | described in US20210179731A1. |
| | VHCDR3 SEQ ID Nos: 16 and 35 described in |
| | US20210179731A1. |
| | VH SEQ ID Nos: 182 or 184 described in |
| | US20210179731A1. |
| | VLCDR1 SEQ ID Nos: 11 or 30 described in |
| | US20210179731A1. |
| | VLCDR2 SEQ ID Nos: 12 or 31 described in |
| | US20210179731A1. |
| | VLCDR3 SEQ ID Nos: 13 or 32 described in |
| | US20210179731A1. |
| | VL SEQ ID Nos: 183 or 186 described in |
| | US20210179731A1. |
| STEAP1 | AMG509 |
| STEAP2 | Anti-STEAP 2 antibodies having CDR-H1, CDR-H2, CDR- |
| | H3, CDR-L1, CDR-L2 and CDR-L3 sequences selected |
| | from SEQ ID NOS: (1) 4-6-8-12-14-16; (2) 20-22-24-28- |
| | 30-32; (3) 36-38-40-44-46-48; (4) 52-54-56-60-62-64; (5) |
| | 68-70-72-60-62-64; (6) 76-78-80-60-62-64; (7) 84-86-88- |
| | 60-62-64; (8) 92-94-96-60-62-64; (9) 100-102-104-60-62- |
| | 64; (10) 108-110-112-116-118-120; (11) 124-126-128- |
| | 132-134-136; (12) 140-142-144-148-150-152; (13) 156- |
| | 158-160-164-166-168; (14) 172-174-176-180-182-184; |
| | (15) 188-190-192-196-198-200; (16) 204-206-208-212- |
| | 214-216; (17) 220-222-224-228-230-232; (18) 236-238- |
| | 240-244-246-248; (19) 252-254-256-260-262-264; (20) |
| | 268-270-272-276-278-280; (21) 284-286-288-292-294- |
| | 296; (22) 300-302-304-308-310-312; (23) 316-318-320- |
| | 324-326-328; (24) 332-334-336-340-342-344; (25) 348- |
| | 350-352-356-358-360; (26) 364-366-368-372-374-376; |
| | and (27) 380-382-384-388-390-392 of U.S. Pat. No. |
| | 10,772,972 B2. |
| | Anti-STEAP 2 antibodies having (a) a VH comprising the |
| | amino acid of any one of SEQ ID Nos: 2, 18, 34, 50, 66, |
| | 74, 82, 90, 98, 106, 122, 138, 154, 170, 186, 202, 218, |
| | 234, 250, 266, 282, 298, 314, 330, 346, 362, and 378 of |
| | U.S. Pat. No. 10,772,972 B2; and (b) a VL comprising |
| | the amino acid sequence of any one of SEQ ID Nos: 10; |
| | 26; 42; 58; 114; 130; 146; 162; 178; 194; 210; 226, 242; |
| | 258; 274; 290; 306; 322; 338; 354; 370; and 386 of U.S. |
| | Pat. No. 10,772,972 B2. |
| | Anti-STEAP 2 antibodies having a VH/VL pair comprising |
| | the amino acid sequences of any of the following pairs of |
| | SEQ ID Nos of U.S. Pat. No. 10,772,972 B2: 2/10; 18/26; |
| | 34/42; 50/58; 66/58; 74/58; 82/58; 90/58; 98/58; 106/114; |
| | 122/130; 138/146; 154/162; 170/178; 186/194; 202/210; |
| | 218/226; 234/242; 250/258; 266/274; 282/290; 298/306; |
| | 314/322; 330/338; 346/354; 362/370; and 378/386. |
| Syndecan-1 (CD 138) | The B-B4 antibody described in Wijdenes et al. (1996) Br. |
| | J. Haematol., 94: 318-323 |
| Syndecan-4 | The amino acid sequence of amino acids 93 and 121 of |
| | SEQ ID NO: 1 or the amino acid sequence of amino acids |
| | 92 and 122 of SEQ ID NO: 2 described in European Patent |
| | Pub. EP 2 603 236. |
| TGFβ | GC1008 |
| TNFR | Enbrel ™ (etanercept) |
| TNFα | Remicade ™ (infliximab) |
| | VH: SEQ ID NO: 2 of Int. Patent Publication |
| | WO201/3087911 A1 |
| | VH: SEQ ID NO: 3 of Int. Patent Publication WO2013/ |
| | A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |
| XCR1 | Anti-XCR1 antibodies disclosed in U.S. Pat. |
| | No. 9,371,389 B2, including: |
| | The antibodies designated 2H6, 5G7, 11H2, |
| | HK1L2 and HK5L5 |
| | Antibodies having CDR-H1, CDR-H2 and CDR-H3 |
| | sequences of SEQ ID Nos: 53-55 and CDR-L1, |
| | CDR-L2 and CDR-L3 sequences of SEQ ID |
| | Nos: 56-58. |
| | Antibodies having CDR-H1, CDR-H2 and CDR-H3 |
| | sequences of SEQ ID Nos: 41-43 and CDR-L1, |
| | CDR-L2 and CDR-L3 sequences of SEQ ID |
| | Nos: 44-46. |

In some embodiments, the targeting moiety specifically binds to PDL1 (e.g., human PDL1). Examples of PDL1 binding antibodies and/or binding sequences are provided in Table A-1, below.

TABLE A-1
Exemplary PDL1 antibodies and/or binding sequences

| Target | Antibody Name and/or Binding Sequences |
|---|---|
| PDL1 | Durvalumab (MEDI4736) |
| | HC: SEQ ID NO: 26 of PCT application No. WO2020225552 |
| | LC: SEQ ID NO: 27 of PCT application No. WO2020225552 |
| PDL1 | Atezolizumab (Tecentriq, MPDL3280A, RG7446) |
| | HC: SEQ ID NO: 20 of U.S. Pat. No. 8,217,149 |
| | LC: SEQ ID NO: 21 of U.S. Pat. No. 8,217,149 |
| PDL1 | MDX 1105 (BMS-936559) |
| PDL1 | Anti-PDL1 antibodies described in U.S. Pat. No. |
| | 7,943,743 as having the following SEQ ID NO. |
| | pairs for heavy and light chain variable domains: |
| | SEQ ID Nos: 1/11 |
| | SEQ ID Nos: 2/12 |
| | SEQ ID Nos: 3/13 |
| | SEQ ID Nos: 4/14 |
| | SEQ ID Nos: 5/15 |
| | SEQ ID Nos: 6/16 |
| | SEQ ID Nos: 7/17 |
| | SEQ ID Nos: 8/18 |
| | SEQ ID Nos: 9/19 |
| | SEQ ID Nos: 10/20 |
| PDL1 | Avelumab, described in U.S. Pat. No.: 9,624,298 as having |
| | the following heavy and light chain variable domains: |
| | HC: SEQ ID NO: 24 |
| | LC: SEQ ID NO: 25 |
| PDL1 | ZKAB001 (Socazolimab) |
| PDL1 | TQB2450 (APL-502 or CBT-502) |
| PDL1 | HLX20 |
| | CDR-H1, CDR-H2 and CDR-H3 sequences of SEQ ID Nos: |
| | 52, 56, and 77 of PCT Pub. No. 2018/080812 |
| | CDR-L1, CDR-L2 and CDR-L3 sequences of SEQ ID Nos: |
| | 65, 42, and 71 of PCT Pub. No. 2018/080812 |
| PDL1 | KN035 (Envafolimab) is a nanobody described as |
| | Hu56V2 in U.S. Pat. No. 11,225,522 as having the |
| | VHH SEQ ID NO: 34 |
| PDL1 | LY3434172 |
| PDL1 | LY3300054 (lodapolimab) described in PCT Pub No: |
| | WO 2017/034916 as having the following heavy and |
| | light chain variable domains: |
| | HC: SEQ ID NO: 10 |
| | L: SEQ ID NO: 11 |
| PDL1 | LDP (lesabelimab, ADG104) described in CN Patent |
| | No: 114225023 as having the following heavy and |
| | light chain variable domains: |
| | HC: SEQ ID NO: 10 |
| | LC: SEQ ID NO: 9 |
| PDL1 | EMB-09 |
| PDL1 | ABL501 |
| PDL1 | INBRX-105 |
| PDL1 | STI-3031 (IMC-001) described in U.S. Pat. No.: |
| | 10,118,963 as having the following heavy and |
| | light chain variable domains: |
| | HC: SEQ ID NO: 1 |
| | LC: SEQ ID NO: 2 |
| PDL1 | BGB-A333 (garivulimab) described in U.S. Pat. No.: |
| | 11,512,132 as having the following heavy and |
| | light chain variable domains: |
| | HC: SEQ ID NO: 22 |
| | LC: SEQ ID NO: 23 |
| PDL1 | HLX301 |
| PDL1 | Y101D |
| PDL1 | ES101 |
| PDL1 | IBI322 |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or |
| | 145 of U.S. Pat. No. 11,208,486. |
| | VL: SEQ ID NO: 24 or 125 of U.S. Pat. No. 11,208,486. |

In some embodiments, the targeting moiety specifically binds to PD1 (e.g., human PD1). Examples of PD1 binding antibodies and/or binding sequences are provided in Table A-2, below.

TABLE A-2
Exemplary PD1 antibodies and/or binding sequences

| Target | Antibody Name and/or Binding Sequences |
|---|---|
| PD1 | MDX-1106/BMS-936558 (nivolumab), a human IgG4 |
| | mAb with the structure described in WHO Drug |
| | Information, Vol. 27, No. 1, pages 68-69 (2013) |
| | and whose heavy and light chain sequences are |
| | disclosed in FIG. 7 of US Pub. No. US20190270812A1 |
| | HC: SEQ ID NO: 23 of US Pub. No. US20190270812A1 |
| | LC: SEQ ID NO: 24 of US Pub. No. US20190270812A1 |
| PD1 | MK-3475 (pembrolizumab), a humanized IgG4 mAb with |
| | the structure described in WHO Drug Information, Vol. |
| | 27, No. 2, pages 161-162 (2013) and whose heavy and |
| | light chain sequences are disclosed in FIG. 6 of |
| | US Pub. No. US20190270812A1 |
| | HC: SEQ ID NO: 21 of US Pub. No. US20190270812A1 |
| | LC: SEQ ID NO: 22 of US Pub. No. US20190270812A1 |
| PD1 | H4H7798N as disclosed in US Pub No. 20150203579 |
| | HC: SEQ ID NO: 330 of US Pub. No. 20150203579 |
| | LC: SEQ ID NO: 331 of US Pub. No. 20150203579 |
| PD1 | Anti-PD1 antibodies having CDR H1-H3 and CDR L1-L3 |
| | sequences corresponding to the following SEQ ID Nos. |
| | of U.S. Pat. No. 11,034,765 B2: |
| | a) SEQ ID Nos: 18, 19, 20, 21, 22, and 23, respectively; |
| | b) SEQ ID Nos: 24, 25, 26, 27, 28, and 29, respectively; |
| | c) SEQ ID Nos: 30, 31, 32, 33, 34, and 35, respectively; |
| | d) SEQ ID Nos: 36, 37, 38, 39, 40, and 41, respectively; |
| | e) SEQ ID Nos: 42, 43, 44, 45, 46, and 47, respectively; |
| | f) SEQ ID Nos: 48, 49, 50, 51, 52, and 53, respectively; |
| | g) SEQ ID Nos: 54, 55, 56, 57, 58, and 59, respectively; and |
| | h) SEQ ID Nos: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 |
| | antibodies having VH/VL pairs having SEQ ID Nos: 2/10, |
| | 18/26, 34/42, 50/58, 66/74, 82/90, 98/106, 1 14/122, |
| | 130/138, 146/154, 162/170, 178/186, 194/202, 210/202, |
| | 218/202, 226/202, 234/202, 242/202, 250/202, 258/202, |
| | 266/202, 274/202, 282/202, 290/202, 298/186, 306/186 |
| | and 314/186 of PCT Pub. WO2015112800A1. |
| PD1 | Anti-PD1 antibodies disclosed in U.S. Pat. No. |
| | 10,294,299 B2 as having the following SEQ ID NO. |
| | pairs for heavy and light chain variable domains: |
| | SEQ ID Nos. 164/178 |
| | SEQ ID Nos. 165/179 |
| | SEQ ID Nos. 166/180 |
| | SEQ ID Nos. 167/181 |
| | SEQ ID Nos. 168/182 |
| | SEQ ID Nos. 169/183 |
| | SEQ ID Nos. 170/184 |
| | SEQ ID Nos. 171/185 |
| | SEQ ID Nos. 172/186 |
| | SEQ ID Nos. 173/187 |
| | SEQ ID Nos. 174/188 |
| | SEQ ID Nos. 175/189 |
| | SEQ ID Nos. 176/190 |
| | SEQ ID Nos. 177/190 |
| PD1 | MEDI-0680 (AMP-514) |
| PD1 | PDR001 (spartalizumab), a humanized IgG4 mAb |
| | whose heavy and light chain sequences are disclosed |
| | as BAP049-Clone-E in U.S. Pat. No.: 9683048 B2. |
| | HC: SEQ ID NO: 91 of U.S. Pat. No.: 9,683,048 |
| | LC: SEQ ID NO: 72 of U.S. Pat. No.: 9,683,048 |
| PD1 | BGB-108 |
| PD1 | h409A11, described in WO2008/156712 |
| | HC: SEQ ID NO: 31 of PCT Pub. WO2008/156712 |
| | LC: SEQ ID NO: 36 of PCT Pub. WO2008/156712 |
| PD1 | h409A16, described in WO2008/156712 |
| | HC: SEQ ID NO: 31 of PCT Pub. WO2008/156712 |
| | LC: SEQ ID NO: 37 of PCT Pub. WO2008/156712 |
| PD1 | h409A17, described in WO2008/156712 |
| | HC: SEQ ID NO: 31 of PCT Pub. WO2008/156712 |
| | LC: SEQ ID NO: 38 of PCT Pub. WO2008/156712 |
| PD1 | Anti-PD1 antibodies described in U.S. Pat. No. 7,488,802 |
| | as having the following SEQ ID NO. pairs for heavy and |
| | light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 7,521,051 |
| | as having the following SEQ ID NO. pairs for heavy and |
| | light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in U.S. Pat. No. 8,008,449 |
| | as having the following SEQ ID NO. pairs for heavy and |
| | light chain variable domains: |
| | SEQ ID Nos. 1/8 |
| | SEQ ID Nos. 2/9 |
| | SEQ ID Nos. 3/10 |
| | SEQ ID Nos. 4/11 |
| | SEQ ID Nos. 5/12 |
| | SEQ ID Nos. 6/13 |
| | SEQ ID Nos. 7/14 |
| PD1 | Anti-PD1 antibodies described in U.S. Pat. No. 8,354,509 |
| | as having the following SEQ ID NO. pairs for heavy and |
| | light chain full-length domains: |
| | SEQ ID Nos. 31/36 |
| | SEQ ID Nos. 31/37 |
| | SEQ ID Nos. 31/38 |
| PD1 | Anti-PD1 antibodies described in U.S. Pat. No. 8,168,757 |
| | as having the following SEQ ID NO. pairs for heavy and |
| | light chain variable domains: |
| | SEQ ID Nos. 4/5 |
| | SEQ ID Nos. 12/13 |
| | SEQ ID Nos. 18/19 |
| | SEQ ID Nos.40/41 |
| | SEQ ID Nos.47/48 |
| | SEQ ID Nos.26/27 |
| | SEQ ID Nos.34/35 |
| | SEQ ID Nos.55/56 |
| | SEQ ID Nos.67/68 |
| PD1 | Anti-PD1 antibodies described in PCT Pub. No. |
| | WO2004/004771 |
| PD1 | Anti-PD1 antibodies described in PCT Pub. No. |
| | WO2004/056875 as having the following SEQ ID NO. |
| | pairs for heavy and light chain variable domains: |
| | SEQ ID Nos. 2/4 |
| | SEQ ID Nos. 6/8 |
| | SEQ ID Nos. 10/12 |
| | SEQ ID Nos. 14/16 |
| | SEQ ID Nos. 47/49 |
| PD1 | Anti-PD1 antibodies described in PCT Pub. No. |
| | WO2004/072286 |
| PD1 | VH: SEQ ID NO: 25, 26, 27, 28, or 29 of US Pub. No. |
| | US2011/0271358 |
| | VL: SEQ ID NO: 30, 31, 32, or 33 of US Pub. No. |
| | US2011/0271358 |
| PD1 | SHR-1210 (Camrelizumab) described in PCT |
| | Publication No: WO |
| | 2015/085847 as having the following heavy and light |
| | chain variable domains: |
| | HC: SEQ ID NO: 9 |
| | LC: SEQ ID NO: 10 |

In some aspects, the targeting moiety competes with an antibody set forth above, including in Table A, Table A-1, or Table A-2 for binding to the target molecule. In further aspects, the targeting moiety comprises CDRs having CDR sequences of an antibody set forth above, including in Table A, Table A-1, or Table A-2. In some embodiments, the targeting moiety comprises all 6 CDR sequences of the antibody set forth above, including the antibody set forth in Table A, Table A-1, or Table A-2. In other embodiments, the targeting moiety comprises at least the heavy chain CDR sequences (CDR-H1, CDR-H2, CDR-H3) of such antibody and the light chain CDR sequences of a universal light chain. In further aspects, a targeting moiety comprises a VH comprising the amino acid sequence of the VH of an antibody set forth above, e.g., in Table A, Table A-1, or Table A-2. In some embodiments, the targeting moiety further comprises a VL comprising the amino acid sequence of the VL of the antibody set forth above, e.g., in Table A, Table A-1, or Table A-2. In other embodiments, the targeting moiety further comprises a universal light chain VL sequence.

Yet additional example target molecules include Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFβR (β-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, β-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) and extra domain B (EDB) of fibronectin and the A1 domain of tenascin-C (TnC A1).

Non-limiting examples of viral antigens include an EBV antigen (e.g., Epstein-Barr virus LMP-1), a hepatitis C virus antigen (e.g., hepatitis C virus E2 glycoprotein), an HIV antigen (e.g., HIV gp160, and HIV gp120); a CMV antigen; a HPV-specific antigen, or an influenza virus antigen (e.g., influenza virus hemagglutinin).

Additional target molecules that can be targeted by the IL18 receptor agonists are disclosed in Table 6 below and in, e.g., Hafeez et al., 2020, Molecules 25:4764, doi: 10.3390/molecules25204764, particularly in Table 1. Table 1 of Hafeez et al. is incorporated by reference in its entirety herein.

### 6.5.1. Targeting Moiety Formats

In certain aspects, the targeting moiety can be any type of antibody or fragment thereof that retains specific binding to an antigenic determinant. In one embodiment the antigen binding moiety is a full-length antibody. In one embodiment the antigen binding moiety is an immunoglobulin molecule, particularly an IgG class immunoglobulin molecule, more particularly an IgG₁ or IgG₄ immunoglobulin molecule. Antibody fragments include, but are not limited to, VH (or V_{H}) fragments, VL (or V_{L}) fragments, Fab fragments, F(ab′)₂ fragments, scFv fragments, Fv fragments, minibodies, diabodies, triabodies, and tetrabodies.

6.5.1.1. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.7.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human, or humanized VH and VL sequences.

To create an scFv-encoding nucleic acid, the VH and VL-encoding DNA fragments are operably linked to another fragment encoding a linker, e.g., encoding any of the linkers described in Section 6.7 (typically a repeat of a sequence containing the amino acids glycine and serine, such as the amino acid sequence (Gly4˜Ser)₃ (SEQ ID NO: 31), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.5.1.2. Fab

Fab domains were traditionally produced by proteolytic cleavage of immunoglobulin molecules using enzymes such as papain. In the IL18 receptor agonists of the disclosure, the Fab domains are typically recombinantly expressed as part of the IL18 receptor agonist.

The Fab domains can comprise constant domain and variable region sequences from any suitable species, and thus can be murine, chimeric, human, or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the IL18 receptor agonists of the disclosure, particularly when the light chain is not a common or universal light chain, it is advantageous to use Fab heterodimerization strategies to permit the correct association of Fab domains belonging to the same ABD and minimize aberrant pairing of Fab domains belonging to different ABDs. For example, the Fab heterodimerization strategies shown in Table H below can be used:

TABLE H
Fab Heterodimerization Strategies

| STRATEGY | VH | CH1 | VL | CL | REFERENCE |
|---|---|---|---|---|---|
| CrossMabC | WT | CL domain | WT | CH1 domain | Schaefer et al., |
| H1-CL | | | | | 2011, Cancer Cell |
| | | | | | 2011; 20: 472-86; |
| | | | | | PMID: 22014573. |
| orthogonal | 39K, | H172A, | 1R, 38D, | L135Y, | Lewis et al., 2014, |
| Fab | 62E | F174G | (36F) | S176W | Nat Biotechnol |
| VHVRD1CH | | | | | 32: 191-8 |
| 1CRD2 - | | | | | |
| VLVRD1Cλ | | | | | |
| CRD2 | | | | | |
| orthogonal | 39Y | WT | 38R | WT | Lewis et al., 2014, |
| Fab | | | | | Nat Biotechnol |
| VHVRD2CH | | | | | 32: 191-8 |
| 1 wt - | | | | | |
| VLVRD2Cλ | | | | | |
| wt | | | | | |
| TCR CαCβ | 39K | TCR Cα | 38D | TCR Cβ | Wu et al., 2015, |
| | | | | | MAbs 7: 364-76 |
| CR3 | WT | T192E | WT | N137K, | Golay at al., 2016, |
| | | | | S114A | J Immunol 196: |
| | | | | | 3199-211. |
| MUT4 | WT | L143Q, | WT | V133T, | Golay at al., 2016, |
| | | S188V | | S176V | J Immunol 196: |
| | | | | | 3199-211. |
| DuetMab | WT | F126C | WT | S121C | Mazor et al., 2015, |
| | | | | | MAbs 7: 377-89; |
| | | | | | Mazor et al., 2015, |
| | | | | | MAbs 7: 461-669. |
| Domain | WT | CH3 + knob | WT | CH3 + hole | Wozniak-Knopp et |
| exchanged | | or hole | | or knob | al., 2018, |
| | | mutation | | mutation | PLoSONE13(4): e01 |
| | | | | | 95442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of an IL18 receptor agonist of the disclosure. In various embodiments, employing a common light chain as described herein reduces the number of inappropriate species of IL18 receptor agonists as compared to employing original cognate VLs. In various embodiments, the VL domains of the IL18 receptor agonists are identified from monospecific antibodies comprising a common light chain. In various embodiments, the VH regions of the IL18 receptor agonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human Vκ1-39Jκ5 sequence or a rearranged human Vκ3-20Jκ1 sequence, and include somatically mutated (e.g., affinity matured) versions. See, for example, U.S. Pat. No. 10,412,940.

### 6.6. Fc Domains

In certain aspects, the IL18 receptor agonists of the disclosure comprise a pair of Fc domains that associate to form an Fc region. In native antibodies, Fc regions comprise hinge regions at their N-termini to form an Fc domain. Throughout this disclosure, the reference to an Fc domain encompasses an Fc domain with a hinge domain at its N-terminus unless specified otherwise.

The Fc domains can be derived from any suitable species operably linked to an ABD or component thereof. In one embodiment the Fc domain is derived from a human Fc domain. In preferred embodiments, an antigen-binding domain of an IL18 receptor agonists of the disclosure is fused to an IgG Fc molecule. An antigen-binding domain may be fused to the N-terminus or the C-terminus of the IgG Fc domain or both.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., IL18 receptor agonists described herein, the Fc domains might advantageously be different to allow for heterodimerization, as described in Section 6.6.2 below. In other embodiments, the two Fc domains of IL18 receptor agonists disclosed herein are the same.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In IL18 receptor agonists of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for IL18 receptor agonists of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild-type constant domain. Preferably the variant constant domains are at least 60% identical or similar to a wild-type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the IL18 receptor agonists of the present disclosure do not comprise a tailpiece.

The Fc domains that are incorporated into the IL18 receptor agonists of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.6.1.

The Fc domains can also be altered to include modifications that improve manufacturability of asymmetric IL18 receptor agonists, for example by allowing heterodimerization, which is the preferential pairing of non-identical Fc domains over identical Fc domains. Heterodimerization permits the production of IL18 receptor agonists in which different polypeptide components are connected to one another by an Fc region containing Fc domains that differ in sequence. Examples of heterodimerization strategies are exemplified in Section 6.6.2.

It will be appreciated that any of the modifications mentioned above can be combined in any suitable manner to achieve the desired functional properties and/or combined with other modifications to alter the properties of the IL18 receptor agonists.

Example Fc domain sequences are provided in Table H-1, below.

TABLE H-1
Fc Sequences

| | | SEQ ID |
|---|---|---|
| Fc | Sequence | NO |
| hIgG1 Fc | EPKSCDKTHTCPPCPAPELLGGPSVFLFPPKPKDTLMISRTPEVTCV | 12 |
| (amino acids 99- | VVDVSHEDPEVKFNWYVDGVEVHNAKTKPREEQYNSTYRVVSVLTVL | |
| 330 of UniprotKB | HQDWLNGKEYKCKVSNKALPAPIEKTISKAKGQPREPQVYTLPPSRD | |
| P01857-1) | ELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| hIgG2 Fc | ERKCCVECPPCPAPPVAGPSVFLFPPKPKDTLMISRTPEVTCVVVDV | 13 |
| (amino acids 99- | SHEDPEVQFNWYVDGVEVHNAKTKPREEQFNSTFRVVSVLTVVHQDW | |
| 326 of UniprotKB | LNGKEYKCKVSNKGLPAPIEKTISKTKGQPREPQVYTLPPSREEMTK | |
| P01859-1) | NQVSLTCLVKGFYPSDISVEWESNGQPENNYKTTPPMLDSDGSFFLY | |
| | SKLTVDKSRWQQGNVFSCSVMHEALHNHYTQKSLSLSPGK | |
| hIgG3 Fc | ELKTPLGDTTHTCPRCPEPKSCDTPPPCPRCPEPKSCDTPPPCPRCP | 14 |
| (amino acids 99- | EPKSCDTPPPCPRCPAPELLGGPSVFLFPPKPKDTLMISRTPEVTCV | |
| 377 of UniprotKB | VVDVSHEDPEVQFKWYVDGVEVHNAKTKPREEQYNSTFRVVSVLTVL | |
| P01860-1) | HQDWLNGKEYKCKVSNKALPAPIEKTISKTKGQPREPQVYTLPPSRE | |
| | EMTKNQVSLTCLVKGFYPSDIAVEWESSGQPENNYNTTPPMLDSDGS | |
| | FFLYSKLTVDKSRWQQGNIFSCSVMHEALHNRFTQKSLSLSPGK | |
| hIgG4 Fc | ESKYGPPCPSCPAPEFLGGPSVFLFPPKPKDTLMISRTPEVTCVVVD | 15 |
| (amino acids 99- | VSQEDPEVQFNWYVDGVEVHNAKTKPREEQFNSTYRVVSVLTVLHQD | |
| 327 of UniprotKB | WLNGKEYKCKVSNKGLPSSIEKTISKAKGQPREPQVYTLPPSQEEMT | |
| P01861-1) | KNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| hIgG4s Fc | ESKYGPPCPPCPAPPVAGPSVFLFPPKPKDTLMISRTPEVTCVVVDV | 16 |
| | SQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLY | |
| | SRLTVDKSRWQEGNVFSCSVMHEALHNHYTQKSLSLSLGK | |
| hIgG1 PVA Fc | EPKSCDKTHTCPPCPAPPVAGPSVFLFPPKPKDTLMISRTPEVTCVV | 17 |
| | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| hIgG1 PVA/P329A | EPKSCDKTHTCPPCPAPPVAGPSVFLFPPKPKDTLMISRTPEVTCVVVDVSH | 134 |
| Fc | EDPEVKFNWYVDGVEVHNAKTKPREEQYNSTYRVVSVLTVLHQDWLNGKEYK | |
| | CKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 LALAPG Fc | EPKSCDKTHTCPPCPAPEAAGGPSVFLFPPKPKDTLMISRTPEVTCVVVDVS | 135 |
| | HEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG1 PVA star: | EPKSCDKTHTCPPCPAPPVAGPSVFLFPPKPKDTLMISRTPEVTCVV | 18 |
| | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSF | |
| | FLYSKLTVDKSRWQQGNVFSCSVMHEALHNRFTQKSLSLSPGK | |
| hIgG1s: | DKKVEPKSCDKTHTCPPCPAPPVAGPSVFLFPPKPKDTLMISRTPEV | 19 |
| | TCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPP | |
| | SRDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPGK | |
| hIgG1us | DKKVEPKSCDKTHTCPPCPAPGGGGPSVFLFPPKPKDTLMISRTPEVTCVVV | 136 |
| | DVSQEDPEVQFNWYVDGVEVHNAKTKPREEQFNSTYRVVSVLTVLHQDWING | |
| | KEYKCKVSNKGLPSSIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| hlgG1 N180G, | EPKSCDKTHTCPPCPAPELLGGPSVFLFPPKPKDTLMISRTPEVTCV | 20 |
| also referred to | VVDVSHEDPEVKFNWYVDGVEVHNAKTKPREEQYGSTYRVVSVLTVL | |
| as N297G: | HQDWLNGKEYKCKVSNKALPAPIEKTISKAKGQPREPQVYTLPPSRD | |
| | ELTKNQVSLTCLVKGFYPSDIAVEWESNGQPENNYKTTPPVLDSDGS | |
| | FFLYSKLTVDKSRWQQGNVFSCSVMHEALHNHYTQKSLSLSPGK | |
| hIgG2 variant | ERKCCVECPPCPAPPVAGPSVFLFPPKPKDTLMISRTPEVTCVVVDV | 21 |
| | SHEDPEVQFNWYVDGVEVHNAKTKPREEQFNSTFRVVSVLTVVHQDW | |
| | LNGKEYKCKVSNKGLPAPIEKTISKTKGQPREPQVYTLPPSREEMTK | |
| | NQVSLTCLVKGFYPSDIAVEWESNGQPENNYKTTPPMLDSDGSFFLY | |
| | SKLTVDKSRWQQGNVFSCSVMHEALHNHYTQKSLSLSPGK | |
| IgG1PVA hinge-Fc | EPKSCDKTHTCPPCPAPPVAGPSVFLFPPKPKDTLMISRTPEVTCVV | 22 |
| knob | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLWCLVKGFYPSDIAVEWESNGQPENNYKTTPPVLDSDGSF | |
| | FLYSKLTVDKSRWQQGNVFSCSVMHEALHNHYTQKSLSLSPGK | |
| IgG1PVA hinge-Fc | EPKSCDKTHTCPPCPAPPVAGPSVFLFPPKPKDTLMISRTPEVTCVV | 23 |
| knob star | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLWCLVKGFYPSDIAVEWESNGQPENNYKTTPPVLDSDGSF | |
| | FLYSKLTVDKSRWQQGNVFSCSVMHEALHNRFTQKSLSLSPGK | |
| IgG1PVA hinge-Fc | EPKSCDKTHTCPPCPAPPVAGPSVFLFPPKPKDTLMISRTPEVTCVV | 24 |
| knob Cys | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPCRDE | |
| | LTKNQVSLWCLVKGFYPSDIAVEWESNGQPENNYKTTPPVLDSDGSF | |
| | FLYSKLTVDKSRWQQGNVFSCSVMHEALHNHYTQKSLSLSPGK | |
| IgG1PVA hinge-Fc | EPKSCDKTHTCPPCPAPPVAGPSVFLFPPKPKDTLMISRTPEVTCVV | 25 |
| knob Cys star | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPCRDE | |
| | LTKNQVSLWCLVKGFYPSDIAVEWESNGQPENNYKTTPPVLDSDGSF | |
| | FLYSKLTVDKSRWQQGNVFSCSVMHEALHNRFTQKSLSLSPGK | |
| IgG1PVA hinge-Fc | EPKSCDKTHTCPPCPAPPVAGPSVFLFPPKPKDTLMISRTPEVTCVV | 26 |
| Hole | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| IgG1PVA hinge-Fc | EPKSCDKTHTCPPCPAPPVAGPSVFLFPPKPKDTLMISRTPEVTCVV | 27 |
| Hole star | VDVSHEDPEVKFNWYVDGVEVHNAKTKPREEQYNSTYRVVSVLTVLH | |
| | QDWLNGKEYKCKVSNKALPAPIEKTISKAKGQPREPQVYTLPPSRDE | |
| | LTKNQVSLSCAVKGFYPSDIAVEWESNGQPENNYKTTPPVLDSDGSF | |
| | FLVSKLTVDKSRWQQGNVFSCSVMHEALHNRFTQKSLSLSPGK | |
| IgG1PVA hinge-Fc | EPKSCDKTHTCPPCPAPPVAGPSVFLFPPKPKDTLMISRTPEVTCVV | 28 |
| Hole Cys | VDVSHEDPEVKFNWYVDGVEVHNAKTKPREEQYNSTYRVVSVLTVLH | |
| | QDWLNGKEYKCKVSNKALPAPIEKTISKAKGQPREPQVCTLPPSRDE | |
| | LTKNQVSLSCAVKGFYPSDIAVEWESNGQPENNYKTTPPVLDSDGSF | |
| | FLVSKLTVDKSRWQQGNVFSCSVMHEALHNHYTQKSLSLSPGK | |
| IgG1PVA hinge-Fc | EPKSCDKTHTCPPCPAPPVAGPSVFLFPPKPKDTLMISRTPEVTCVV | 29 |
| Hole Cys star | VDVSHEDPEVKFNWYVDGVEVHNAKTKPREEQYNSTYRVVSVLTVLH | |
| | QDWLNGKEYKCKVSNKALPAPIEKTISKAKGQPREPQVCTLPPSRDE | |
| | LTKNQVSLSCAVKGFYPSDIAVEWESNGQPENNYKTTPPVLDSDGSF | |
| | FLVSKLTVDKSRWQQGNVFSCSVMHEALHNRFTQKSLSLSPGK | |
| hIgG4 S108P | ESKYGPPCPPCPAPEFLGGPSVFLFPPKPKDTLMISRTPEVTCVVVD | 30 |
| | VSQEDPEVQFNWYVDGVEVHNAKTKPREEQFNSTYRVVSVLTVLHQD | |
| | WLNGKEYKCKVSNKGLPSSIEKTISKAKGQPREPQVYTLPPSQEEMT | |
| | KNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to any one of the sequences disclosed in Table H-1.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 12. In cases where an Fc domain comprises at least 90% sequence identity and less than 100% sequence identity to SEQ ID NO: 12 (e.g., between 90% and 99% sequence identity to SEQ ID NO: 12), an Fc domain may also comprise one or more amino acid substitutions described herein, for example one or more substitutions that reduce effector function (e.g., as described in Section 6.6.1) and/or one or more substitutions that promote Fc heterodimerization (e.g., as described in Section 6.6.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:13. In cases where an Fc domain comprises at least 90% sequence identity and less than 100% sequence identity to SEQ ID NO: 13 (e.g., between 90% and 99% sequence identity to SEQ ID NO:13), an Fc domain may also comprise one or more amino acid substitutions described herein, for example one or more substitutions that reduce effector function (e.g., as described in Section 6.6.1) and/or one or more substitutions that promote Fc heterodimerization (e.g., as described in Section 6.6.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 14. In cases where an Fc domain comprises at least 90% sequence identity and less than 100% sequence identity to SEQ ID NO: 14 (e.g., between 90% and 99% sequence identity to SEQ ID NO: 14), an Fc domain may also comprise one or more amino acid substitutions described herein, for example one or more substitutions that reduce effector function (e.g., as described in Section 6.6.1) and/or one or more substitutions that promote Fc heterodimerization (e.g., as described in Section 6.6.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:15. In cases where an Fc domain comprises at least 90% sequence identity and less than 100% sequence identity to SEQ ID NO: 15 (e.g., between 90% and 99% sequence identity to SEQ ID NO:15), an Fc domain may also comprise one or more amino acid substitutions described herein, for example one or more substitutions that reduce effector function (e.g., as described in Section 6.6.1) and/or one or more substitutions that promote Fc heterodimerization (e.g., as described in Section 6.6.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 16.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:17.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 18.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 19.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:20.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:21.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:22.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:23.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:24.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:25.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:26.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:27.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:28.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:29.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:30.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:134.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:135.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:136.

6.6.1. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IL18 receptor agonist) or the Fc region (e.g., one or both Fc domains of an IL18 receptor agonist that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H-2 below: In some embodiments, the Fc domain includes only the bolded portion of the sequences shown below:

TABLE H-2

| Fc Domain | Sequence | SEQ ID NO: |
|---|---|---|
| SEQ ID NO: 1 of | DKRVESKYGP PCPPCPAPPV AGPSVFLFPP | 32 |
| WO2014/121087 | KPKDTLMISR TPEVTCVVVD VSQEDPEVQF | |
| | NWYVDGVEVH NAKTKPREEQ FNSTYRVVSV | |
| | LTVLHQDWLN GKEYKCKVSN KGLPSSIEKT | |
| | ISKAKGQPRE PQVYTLPPSQ EEMTKNQVSL | |
| | TCLVKGFYPS DIAVEWESNG QPENNYKTTP | |
| | PVLDSDGSFF LYSRLTVDKS | |
| | RWQEGNVFSC SVMHEALHNH YTQKSLSLSL GK | |
| SEQ ID NO: 2 of | DKKVEPKSCD KTHTCPPCPA PPVAGPSVFL | 19 |
| WO2014/121087 | FPPKPKDTLM ISRTPEVTCV VVDVSQEDPE | |
| | VQFNWYVDGV EVHNAKTKPR EEQFNSTYRV | |
| | VSVLTVLHQD WLNGKEYKCK VSNKGLPSSI | |
| | EKTISKAKGQ PREPQVYTLP PSRDELTKNQ | |
| | VSLTCLVKGF YPSDIAVEWE SNGQPENNYK | |
| | TTPPVLDSDG SFFLYSKLTV | |
| | DKSRWQQGNV FSCSVMHEAL HNHYTQKSLS | |
| | LSPGK | |
| SEQ ID NO: 30 of | ASTKGPSVFP LAPSSKSTSG GTAALGCLVK | 33 |
| WO2014/121087 | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPPVAGP | |
| | SVFLFPPKPK DTLMISRTPE VTCVVVDVSQ | |
| | EDPEVQFNWY VDGVEVHNAK TKPREEQFNS | |
| | TYRVVSVLTV LHQDWLNGKE | |
| | YKCKVSNKGL PSSIEKTISK AKGQPREPQV | |
| | YTLPPSRDEL TKNQVSLTCL VKGFYPSDIA | |
| | VEWESNGQPE NNYKTTPPVL DSDGSFFLYS | |
| | KLTVDKSRWQ QGNVFSCSVM HEALHNHYTQ | |
| | KSLSLSPGK | |
| SEQ ID NO: 31 of | ASTKGPSVFP LAPCSRSTSE STAALGCLVK | 34 |
| WO2014/121087 | DYFPEPVTVS WNSGALTSGV HTFPAVLQSS | |
| | GLYSLSSVVT VPSSSLGTKT YTCNVDHKPS | |
| | NTKVDKRVES KYGPPCPPCP APPVAGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKC | |
| | KVSNKGLPSS IEKTISKAKG QPREPQVYTL | |
| | PPSQEEMTKN QVSLTCLVKG FYPSDIAVEW | |
| | ESNGQPENNY KTTPPVLDSD GSFFLYSRLT | |
| | VDKSRWQEGN VFSCSVMHEA LHNHYTQKSL | |
| | SLSLGK | |
| SEQ ID NO: 37 of | ASTKGPSVFP LAPSSKSTSG GTAALGCLVK | 35 |
| WO2014/121087 | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPPVAGP | |
| | SVFLFPPKPK DTLMISRTPE VTCVVVDVSQ | |
| | EDPEVQFNWY VDGVEVHNAK TKPREEQFNS | |
| | TYRVVSVLTV LHQDWLNGKE | |
| | YKCKVSNKGL PSSIEKTISK AKGQPREPQV | |
| | YTLPPSRDEL KNQVSLTCL VKGFYPSDIA | |
| | VEWESNGQPE NNYKTTPPVL DSDGSFFLYS | |
| | KLTVDKSRWQ QGNVFSCSVM HEALHNRFTQ | |
| | KSLSLSPGK | |
| SEQ ID NO: 38 of | ASTKGPSVFP LAPCSRSTSE STAALGCLVK | 36 |
| WO2014/121087 | DYFPEPVTVS WNSGALTSGV HTFPAVLQSS | |
| | GLYSLSSVVT VPSSSLGTKT YTCNVDHKPS | |
| | NTKVDKRVES KYGPPCPPCP APPVAGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKC | |
| | KVSNKGLPSS IEKTISKAKG QPREPQVYTL | |
| | PPSQEEMTKN QVSLTCLVKG FYPSDIAVEW | |
| | ESNGQPENNY KTTPPVLDSD GSFFLYSRLT | |
| | VDKSRWQEGN VFSCSVMHEA LHNRFTQKSL | |
| | SLSLGK | |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.6.2. Fc Heterodimerization Variants

Certain IL18 receptor agonists entail dimerization between two Fc domains that, unlike a native immunoglobulin, are operably linked to non-identical N-terminal or C-terminal regions. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the IL18 receptor agonists of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

In some embodiments, the present disclosure provides IL18 receptor agonists comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine(S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL18 receptor agonists comprise a first CH3 domain and a second Ig CH3 domain, wherein the first and second Ig CH3 domains differ from one another by at least one amino acid, and wherein at least one amino acid difference reduces binding of the IL18 receptor agonists to Protein A as compared to a corresponding IL18 receptor agonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.6.3. Hinge Domains

The IL18 receptor agonists of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric IL18 receptor agonist formed by the association of two Fc domains) can comprise two associated hinge sequences on separate polypeptide chains. Sometimes, the two associated hinge sequences are referred to as a “hinge region”. In certain embodiments of IL18 receptor agonists of the disclosure, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama, or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an IL18 receptor agonist of the disclosure comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the IL18 receptor agonists of the disclosure comprise a modified hinge region that reduces binding affinity for an Fcγ receptor relative to a wild-type hinge region of the same isotype (e.g., human IgG1 or human IgG4).

In one embodiment, the IL18 receptor agonists of the disclosure comprise an Fc region in which each Fc domain possesses an intact hinge domain at its N-terminus, where each Fc domain and hinge domain is derived from IgG4 and each hinge domain comprises the modified sequence CPPC (SEQ ID NO: 37). The core hinge region of human IgG4 contains the sequence CPSC (SEQ ID NO: 38) compared to IgG1 that contains the sequence CPPC (SEQ ID NO: 37). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.6.3.1. Chimeric Hinge Sequences

The hinge domain can be a chimeric hinge domain. A “chimeric” hinge domain describes a hinge domain comprising a first region from a first type of IgG (e.g., IgG1, IgG2, IgG3, or IgG4), and a second region from a second, different type of IgG (e.g., IgG1, IgG2, IgG3, or IgG4).

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 39) (previously disclosed as SEQ ID NO: 8 of WO 2014/121087, which is incorporated by reference in its entirety herein) or ESKYGPPCPPCPAPPVA (SEQ ID NO: 40) (previously disclosed as SEQ ID NO:9 of WO 2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1).

6.6.3.2. Hinge Sequences with Reduced Effector Function

In further embodiments, the hinge region can be modified to reduce effector function, for example as described in WO 2016/161010 A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO 2016161010 A2). These segments can be represented as GGG-, GG--, G--- or --- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO 2016/161010 A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 41) (previously disclosed as SEQ ID NO:1 of WO2016/161010A2), CPPCPAPGG--GPSVF (SEQ ID NO: 42) (previously disclosed as SEQ ID NO:2 of WO 2016/161010 A2), CPPCPAPG---GPSVF (SEQ ID NO: 43) (previously disclosed as SEQ ID NO:3 of WO 2016/161010 A2), or CPPCPAP---GPSVF (SEQ ID NO: 44) (previously disclosed as SEQ ID NO:4 of WO 2016/161010 A2).

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO 2016/161010 A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.6.1).

### 6.7. Linkers

In certain aspects, the present disclosure provides IL18 receptor agonists in which two or more components of an IL18 receptor agonist are connected to one another by a peptide linker. By way of example and not limitation, linkers can be used to connect (a) an IL18 moiety and an Fc domain; (b) an IL18 moiety and an IL18Rα moiety; (c) a targeting moiety (e.g., a Fab domain) and an Fc domain; (d) an IL18 moiety and an Fc domain; (e) an IL18Rα moiety and an Fc domain; or (f) different domains within a targeting moiety (e.g., the VH and VL domains in a scFv).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the IL18 receptor agonists of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO: 45) or SGₙ (SEQ ID NO: 46), where n is an integer from 1 to 10, e.g., 12, 3, 4, 5, 6, 7, 8, 9 or 10. In one embodiment, the linker is or comprises a monomer or multimer of repeat of G₄S (SEQ ID NO: 47) e.g., (GGGGS)ₙ (SEQ ID NO: 47).

Polyglycine linkers can suitably be used in the IL18 receptor agonists of the disclosure. In some embodiments, a peptide linker comprises two consecutive glycines (2Gly), three consecutive glycines (3Gly), four consecutive glycines (4Gly) (SEQ ID NO: 48), five consecutive glycines (5Gly) (SEQ ID NO: 49), six consecutive glycines (6Gly) (SEQ ID NO: 50), seven consecutive glycines (7Gly) (SEQ ID NO: 51), eight consecutive glycines (8Gly) (SEQ ID NO: 52) or nine consecutive glycines (9Gly) (SEQ ID NO: 53).

Exemplary linker sequences are set forth in Table L below. An IL18 receptor agonist of the disclosure may comprise one or more linkers of Table L.

TABLE L
Linker Sequences

| | | SEQ |
|---|---|---|
| Linker Sequence | Designation | ID NO: |
| (GGGGS)ₙ | L1 | 47 |
| (GGGS)ₙ | L2 | 54 |
| (GGS)ₙ | L3 | N/A |
| (GS)ₙ | L4 | N/A |
| (GSGGS)ₙ | L5 | 55 |
| AAAGG | L6 | 56 |
| ADAAP | L7 | 57 |
| ADAAPTVSIFP | L8 | 58 |
| ADAAPTVSIFPP | L9 | 59 |
| AKTTAP | L10 | 60 |
| AKTTAPSVYPLAP | L11 | 61 |
| AKTTPKLEEGEFSEARV | L12 | 62 |
| AKTTPKLGG | L13 | 63 |
| AKTTPP | L14 | 64 |
| AKTTPPSVTPLAP | L15 | 65 |
| ASTKGP | L16 | 66 |
| ASTKGPSVFPLAPASTKGPSVFPLAP | L17 | 67 |
| EGKSSGSGSESKST | L18 | 68 |
| GEGESGEGESGEGES | L19 | 69 |
| GEGESGEGESGEGESGEGES | L20 | 70 |
| GEGGSGEGGSGEGGS | L21 | 71 |
| GENKVEYAPALMALS | L22 | 72 |
| GGEGSGGEGSGGEGS | L23 | 73 |
| GGGESGGEGSGEGGS | L24 | 74 |
| GGGESGGGESGGGES | L25 | 75 |
| GGGGSGGGGS | L26 | 76 |
| GGGGSGGGGSGGGGS | L27 | 31 |
| GGGGSGGGGSGGGGSGGGGS | L28 | 77 |
| GGGKSGGGKSGGGKS | L29 | 78 |
| GGGKSGGKGSGKGGS | L30 | 79 |
| GGGS | L31 | 54 |
| GGGSG | L32 | 80 |
| GGKGSGGKGSGGKGS | L33 | 81 |
| GGS | L34 | N/A |
| GGSG | L35 | 82 |
| GGSGG | L36 | 83 |
| GGSGG | L37 | 83 |
| GGSGGGGSG | L38 | 84 |
| GGSGGGGGGGGS | L39 | 85 |
| GHEAAAVMQVQYPAS | L40 | 86 |
| GKGGSGKGGSGKGGS | L41 | 87 |
| GKGKSGKGKSGKGKS | L42 | 88 |
| GKGKSGKGKSGKGKSGKGKS | L43 | 89 |
| GKPGSGKPGSGKPGS | L44 | 90 |
| GKPGSGKPGSGKPGSGKPSGS | L45 | 91 |
| GPAKELTPLKEAKVS | L46 | 92 |
| GSAGSAAGSGEF | L47 | 93 |
| GSGGG | L48 | 94 |
| GSGSG | L49 | 95 |
| GSS | L50 | N/A |
| GSSG | L51 | 96 |
| GSSGGSGGSG | L52 | 97 |
| GSSGGSGGSGG | L53 | 98 |
| GSSGGSGGSGGS | L54 | 99 |
| GSSGGSGGSGGSG | L55 | 100 |
| GSSGGSGGSGGSGGGS | L56 | 10 |
| GSSGGSGGSGS | L57 | 102 |
| GSSGT | L58 | 103 |
| GSSSG | L59 | 104 |
| GSTSGSGKPGSGEGSTKG | L60 | 105 |
| GTAAAGAGAAGGAAAGAAG | L61 | 106 |
| GTSGSSGSGSGGSGSGGGG | L62 | 107 |
| IRPRAIGGSKPRVA | L63 | 108 |
| KESGSVSSEQLAQFRSLD | L64 | 109 |
| KTTPKLEEGEFSEAR | L65 | 110 |
| PRGASKSGSASQTGSAPGS | L66 | 111 |
| QPKAAP | L67 | 112 |
| QPKAAPSVTLFPP | L68 | 113 |
| RADAAAA(G4S)4 | L69 | 114 |
| RADAAAAGGPGS | L70 | 115 |
| RADAAP | L71 | 116 |
| RADAAPTVS | L72 | 117 |
| SAKTTP | L73 | 118 |
| SAKTTPKLEEGEFSEARV | L74 | 119 |
| SAKTTPKLGG | L75 | 120 |
| STAGDTHLGGEDFD | L76 | 121 |
| TVAAP | L77 | 122 |
| TVAAPSVFIFPP | L78 | 123 |
| TVAAPSVFIFPPTVAAPSVFIFPP | L79 | 124 |

In some embodiments, the IL18 receptor agonist comprises linker L1. In some embodiments, the IL18 receptor agonist comprises linker L2. In some embodiments, the IL18 receptor agonist comprises linker L3. In some embodiments, the IL18 receptor agonist comprises linker L4. In some embodiments, the IL18 receptor agonist comprises linker L5. In some embodiments, the IL18 receptor agonist comprises linker L6. In some embodiments, the IL18 receptor agonist comprises linker L7. In some embodiments, the IL18 receptor agonist comprises linker L8. In some embodiments, the IL18 receptor agonist comprises linker L9. In some embodiments, the IL18 receptor agonist comprises linker L10. In some embodiments, the IL18 receptor agonist comprises linker L11. In some embodiments, the IL18 receptor agonist comprises linker L12. In some embodiments, the IL18 receptor agonist comprises linker L13. In some embodiments, the IL18 receptor agonist comprises linker L14. In some embodiments, the IL18 receptor agonist comprises linker L15. In some embodiments, the IL18 receptor agonist comprises linker L16. In some embodiments, the IL18 receptor agonist comprises linker L17. In some embodiments, the IL18 receptor agonist comprises linker L18. In some embodiments, the IL18 receptor agonist comprises linker L19. In some embodiments, the IL18 receptor agonist comprises linker L20. In some embodiments, the IL18 receptor agonist comprises linker L21. In some embodiments, the IL18 receptor agonist comprises linker L22. In some embodiments, the IL18 receptor agonist comprises linker L23. In some embodiments, the IL18 receptor agonist comprises linker L24. In some embodiments, the IL18 receptor agonist comprises linker L25. In some embodiments, the IL18 receptor agonist comprises linker L26. In some embodiments, the IL18 receptor agonist comprises linker L27. In some embodiments, the IL18 receptor agonist comprises linker L28. In some embodiments, the IL18 receptor agonist comprises linker L29. In some embodiments, the IL18 receptor agonist comprises linker L30. In some embodiments, the IL18 receptor agonist comprises linker L31. In some embodiments, the IL18 receptor agonist comprises linker L32. In some embodiments, the IL18 receptor agonist comprises linker L33. In some embodiments, the IL18 receptor agonist comprises linker L34. In some embodiments, the IL18 receptor agonist comprises linker L35. In some embodiments, the IL18 receptor agonist comprises linker L36. In some embodiments, the IL18 receptor agonist comprises linker L37. In some embodiments, the IL18 receptor agonist comprises linker L38. In some embodiments, the IL18 receptor agonist comprises linker L39. In some embodiments, the IL18 receptor agonist comprises linker L40. In some embodiments, the IL18 receptor agonist comprises linker L41. In some embodiments, the IL18 receptor agonist comprises linker L42. In some embodiments, the IL18 receptor agonist comprises linker L43. In some embodiments, the IL18 receptor agonist comprises linker L44. In some embodiments, the IL18 receptor agonist comprises linker L45. In some embodiments, the IL18 receptor agonist comprises linker L46. In some embodiments, the IL18 receptor agonist comprises linker L47. In some embodiments, the IL18 receptor agonist comprises linker L48. In some embodiments, the IL18 receptor agonist comprises linker L49. In some embodiments, the IL18 receptor agonist comprises linker L50. In some embodiments, the IL18 receptor agonist comprises linker L51. In some embodiments, the IL18 receptor agonist comprises linker L52. In some embodiments, the IL18 receptor agonist comprises linker L53. In some embodiments, the IL18 receptor agonist comprises linker L54. In some embodiments, the IL18 receptor agonist comprises linker L55. In some embodiments, the IL18 receptor agonist comprises linker L56. In some embodiments, the IL18 receptor agonist comprises linker L57. In some embodiments, the IL18 receptor agonist comprises linker L58. In some embodiments, the IL18 receptor agonist comprises linker L59. In some embodiments, the IL18 receptor agonist comprises linker L60. In some embodiments, the IL18 receptor agonist comprises linker L61. In some embodiments, the IL18 receptor agonist comprises linker L62. In some embodiments, the IL18 receptor agonist comprises linker L63. In some embodiments, the IL18 receptor agonist comprises linker L64. In some embodiments, the IL18 receptor agonist comprises linker L65. In some embodiments, the IL18 receptor agonist comprises linker L66. In some embodiments, the IL18 receptor agonist comprises linker L67. In some embodiments, the IL18 receptor agonist comprises linker L68. In some embodiments, the IL18 receptor agonist comprises linker L69. In some embodiments, the IL18 receptor agonist comprises linker L70. In some embodiments, the IL18 receptor agonist comprises linker L71. In some embodiments, the IL18 receptor agonist comprises linker L72. In some embodiments, the IL18 receptor agonist comprises linker L73. In some embodiments, the IL18 receptor agonist comprises linker L74. In some embodiments, the IL18 receptor agonist comprises linker L75. In some embodiments, the IL18 receptor agonist comprises linker L76. In some embodiments, the IL18 receptor agonist comprises linker L77. In some embodiments, the IL18 receptor agonist comprises linker L78. In some embodiments, the IL18 receptor agonist comprises linker L79.

### 6.8. Nucleic Acids and Host Cells

In another aspect, the disclosure provides nucleic acids encoding the IL18 receptor agonists of the disclosure. In some embodiments, the IL18 receptor agonists are encoded by a single nucleic acid. In other embodiments, for example in the case of a heterodimeric molecule or a molecule comprising a targeting moiety composed of more than one polypeptide chain, the IL18 receptor agonists can be encoded by a plurality (e.g., two, three, four or more) nucleic acids.

A single nucleic acid can encode an IL18 receptor agonist that comprises a single polypeptide chain, an IL18 receptor agonist that comprises two or more polypeptide chains, or a portion of an IL18 receptor agonist that comprises more than two polypeptide chains (for example, a single nucleic acid can encode two polypeptide chains of an IL18 receptor agonist comprising three, four or more polypeptide chains, or three polypeptide chains of an IL18 receptor agonist comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, an IL18 receptor agonist comprising two or more polypeptide chains is encoded by two or more nucleic acids. The number of nucleic acids encoding an IL18 receptor agonist can be equal to or less than the number of polypeptide chains in the IL18 receptor agonist (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.8.1. Vectors

The disclosure provides vectors comprising nucleotide sequences encoding an IL18 receptor agonist or an IL18 receptor agonist component described herein, for example one or two of the polypeptide chains of a half antibody. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.8.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 6.9. Pharmaceutical Compositions

The IL18 receptor agonists of the disclosure may be in the form of compositions comprising the IL18 receptor agonist and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the IL18 receptor agonist and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an IL18 receptor agonist of the disclosure per dose. The quantity of IL18 receptor agonist included in a unit dose will depend on the disease being treated, as well as other factors as are well known in the art. Such unit dosages may be in the form of a lyophilized dry powder containing an amount of IL18 receptor agonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of IL18 receptor agonist suitable for a single administration.

The pharmaceutical compositions may also be supplied in bulk from containing quantities of IL18 receptor agonist suitable for multiple administrations.

Pharmaceutical compositions may be prepared for storage as lyophilized formulations or aqueous solutions by mixing an IL18 receptor agonist having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/V). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IL18 receptor agonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.10. Therapeutic Indications and Methods of Treatment

The present disclosure provides methods for using and applications for the IL18 receptor agonists of the disclosure.

IL18 receptor agonists of the disclosure are useful in treating disease states where stimulation of the immune system of the host is beneficial, in particular conditions where an enhanced cellular immune response is desirable. These may include disease states where the host immune response is insufficient or deficient.

Disease states for which the IL18 receptor agonists of the disclosure can be administered comprise, for example, a tumor or infection where a cellular immune response would be a critical mechanism for specific immunity. Specific disease states for which IL18 receptor agonists of the present disclosure can be employed include cancer. The IL18 receptor agonists of the disclosure may be administered per se or in any suitable pharmaceutical composition.

In one aspect, IL18 receptor agonists of the disclosure for use as a medicament are provided. In further aspects, IL18 receptor agonists of the disclosure for use in treating a disease are provided. In certain embodiments, IL18 receptor agonists of the disclosure for use in a method of treatment are provided. In one embodiment, the disclosure provides an IL18 receptor agonist as described herein for use in the treatment of a disease in a subject in need thereof. In certain embodiments, the disclosure provides an IL18 receptor agonist for use in a method of treating a subject having a disease comprising administering to the individual a therapeutically effective amount of the IL18 receptor agonist. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent such as a chemotherapeutic or immunotherapeutic, if the disease to be treated is cancer. In further embodiments, the disclosure provides an IL18 receptor agonist for use in stimulating the immune system. In certain embodiments, the disclosure provides an IL18 receptor agonist for use in a method of stimulating the immune system in a subject comprising administering to the individual an effective amount of the IL18 receptor agonist to stimulate the immune system. An “individual” according to any of the above embodiments is a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL18 receptors, an increase in T cell responsiveness, an increase in natural killer cell activity or lymphokine-activated killer (LAK) cell activity, and the like.

In a further aspect, the disclosure provides for the use of an IL18 receptor agonist of the disclosure in the manufacture or preparation of a medicament for the treatment of a disease in a subject in need thereof. In one embodiment, the medicament is for use in a method of treating a disease comprising administering to a subject having the disease a therapeutically effective amount of the medicament. In certain embodiments the disease to be treated is a proliferative disorder. In a preferred embodiment the disease is cancer. In one such embodiment, the method further comprises administering to the individual a therapeutically effective amount of at least one additional therapeutic agent, e.g., an anti-cancer agent such as a chemotherapeutic or immunotherapeutic, if the disease to be treated is cancer. In a further embodiment, the medicament is for stimulating the immune system. In a further embodiment, the medicament is for use in a method of stimulating the immune system in a subject comprising administering to the individual an amount effective of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL18 receptors, an increase in T cell responsiveness, an increase in natural killer cell activity or lymphokine-activated killer (LAK) cell activity, and the like.

In a further aspect, the disclosure provides a method for treating a disease in a subject, comprising administering to said individual a therapeutically effective amount of an IL18 receptor agonist of the disclosure. In one embodiment a composition is administered to said individual, comprising the IL18 receptor agonist of the disclosure in a pharmaceutically acceptable form. In certain embodiments the disease to be treated is a proliferative disorder. In a preferred embodiment the disease is cancer. In certain embodiments the method further comprises administering to the individual a therapeutically effective amount of at least one additional therapeutic agent, e.g., an anti-cancer agent such as a chemotherapeutic or immunotherapeutic, if the disease to be treated is cancer. In a further aspect, the disclosure provides a method for stimulating the immune system in a subject, comprising administering to the individual an effective amount of an IL18 receptor agonist to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL18 receptors, an increase in T cell responsiveness, an increase in natural killer cell activity or lymphokine-activated killer (LAK) cell activity, and the like.

In certain aspects, the disclosure provides a method of treating cancer, comprising administering to a subject in need thereof an IL18 receptor agonist or pharmaceutical composition as described herein.

In some embodiments, the disclosure provides a method of treating cancer with an IL18 receptor agonist protein that is targeted to cancer tissue, comprising administering to a subject in need thereof an IL18 receptor agonist or pharmaceutical composition as described herein, where the IL18 receptor agonist comprises a targeting moiety that recognizes a target molecule that is expressed in the tumor tissue (e.g., the cancer cells, the extracellular matrix, tumor lymphocytes, etc.).

The present disclosure further provides a method of localized delivery of an IL18 protein, comprising administering to a subject an IL18 receptor agonist or pharmaceutical composition as described herein, where the IL18 receptor agonist comprises a targeting moiety that recognizes a target molecule that is expressed by a tissue to which the IL18 receptor agonist is to be locally delivered. As used herein, the term “locally delivered” does not require local administration but rather indicates that the IL18 receptor agonist be selectively localized to a tissue of interest following administration.

The present disclosure further provides a method of administering to the subject IL18 therapy with reduced systemic exposure and/or reduced systemic toxicity, comprising administering to a subject the IL18 therapy in the form of an IL18 receptor agonist or pharmaceutical composition as described herein. Accordingly, the foregoing methods permit IL18 therapy with reduced off-target side effects by virtue of preferential targeting of an IL18 receptor agonist to a particular target tissue and/or attenuation and/or masking of the IL18 moiety until at the site of intended activity.

The present disclosure further provides method of locally inducing an immune response in a target tissue, comprising administering to a subject IL18 receptor agonist or pharmaceutical composition as described herein which has one or more targeting moieties capable of binding a target molecule expressed in the target tissue. The IL18 receptor agonist can then induce the immune response against at least one cell type in the target tissue.

In some embodiments, the administration is not local to the tissue. For example, when the target tissue is cancer tissue, the administration can be systemic or subcutaneous.

In certain embodiments the disease to be treated is a proliferative disorder, preferably cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using an IL18 receptor agonist of the present disclosure include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer.

Similarly, other cell proliferation disorders can also be treated by the IL18 receptor agonists of the present disclosure. Examples of such cell proliferation disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

Table I below shows exemplary indications for which IL18 receptor agonists targeting particular target molecules can be used.

TABLE I
Examples of Target Molecule Indications

| Target | Exemplary Indication(s) |
|---|---|
| ADRB3 | Ewing sarcoma |
| ALK | NSCLC, ALCL, IMT, neuroblastoma |
| B7H3 | melanoma, osteosarcoma, leukemia, breast, |
| | prostate, ovarian, pancreatic, colorectal cancers |
| BCMA | multiple myeloma, leukemia (e.g., acute |
| | lymphoblastic leukemia (“ALL”), acute myeloid |
| | leukemia (“AML”), chronic lymphocytic leukemia |
| | (“CLL”), chronic myeloid leukemia (“CML”) and |
| | hairy cell leukemia (“HCL”)); |
| | lymphoma (e.g., Hodgkin's lymphoma, non-Hodgkin's |
| | lymphoma, including diffuse large B-cell lymphoma |
| | (“DLBCL”)) |
| Cadherin 17 | gastric, pancreatic, and colorectal adenocarcinomas |
| CAIX | clear-cell renal cell carcinoma, hypoxic solid |
| | tumors, head and neck squamous carcinoma |
| CD123 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma. In |
| | a preferred embodiment, the indication is AML. |
| CD171 | neuroblastoma, paraganglioma |
| CD179a | B cell malignancies |
| CD19 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma. |
| CD20 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma. |
| CD22 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma; |
| | lung cancer |
| CD24 | ovarian, breast, prostate, bladder, renal, |
| | non-small cell carcinomas |
| CD30 | anaplastic large cell lymphoma, embryonal |
| | carcinoma, Hodgkin Lymphoma |
| CD32b | B cell malignancies, gastric, pancreatic, |
| | esophageal, glioblastoma, breast, colorectal |
| CD33 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma. In |
| | a preferred embodiment, the indication is AML. |
| CD38 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma |
| CD44v6 | colon cancer, head and neck small cell carcinoma |
| CD97 | B cell malignancies, gastric, pancreatic, |
| | esophageal, glioblastoma, breast, colorectal |
| CEA | colorectal carcinoma, gastric carcinoma, |
| (CEACAM5) | pancreatic carcinoma, lung cancer, breast |
| | cancer, medullary thyroid carcinoma |
| CLDN6 | ovarian, breast, lung cancer |
| CLL-1 | leukemia (e.g., ALL, CLL, AML, CML, HCL); |
| | lymphoma (e.g., Hodgkin's lymphoma, non-Hodgkin's |
| | lymphoma, e.g., DLBCL); multiple myeloma. In |
| | a preferred embodiment, the indication is AML. |
| CS1 (SLAMF7) | multiple myeloma |
| EGFR | squamous cell carcinoma of lung, anal cancer, |
| | glioblastoma, epithelial tumors of head and neck, |
| | colon cancer |
| EGFRvIII | Glioblastoma |
| EPCAM | gastrointestestinal carcinoma, colorectal cancer |
| EphA2 | kaposi's sarcoma, glioblastoma, solid tumors, glioma |
| Ephrin B2 | thyroid cancer, breast cancer, malignant melanoma |
| ERBB2 | breast, ovarian, gastric cancers, lung |
| (Her2/neu) | adenocarcinoma, non-small cell lung cancer, |
| | uterine cancer, uterine serous endometrial |
| | carcinoma, salivary duct carcinoma |
| FAP | pancreatic cancer, colorectal cancer, metastasis, |
| | epithelial cancers, soft tissue sarcomas |
| FCRL5 | multiple myeloma |
| FLT3 | leukemia (e.g., ALL, CLL, AML, CML, HCL), |
| | lymphoma (e.g., Hodgkin's lymphoma, non-Hodgkin's |
| | lymphoma, e.g., DLBCL), multiple myeloma |
| Folate | ovarian, breast, renal, lung, colorectal, |
| receptor alpha | brain cancers |
| Folate | ovarian cancer |
| receptor beta | |
| Fucosyl GM1 | AML, myeloma |
| GD2 | malignant melanoma, neuroblastoma |
| GD3 | Melanoma |
| GloboH | ovarian, gastric, prostate, lung, breast, |
| | and pancreatic cancers |
| gp100 | Melanoma |
| GPNMB | breast cancer, head and neck cancers |
| GPR20 | GIST |
| GPR64 | Ewing sarcoma, prostate, kidney and lung sarcomas |
| GPRC5D | multiple myeloma |
| HAVCR1 | renal cancer |
| HER2 | HER-2 (+) adenocarcinoma of gastroesophageal |
| | junction, HER-2 positive gastric adenocarcinoma, |
| | HER2 positive carcinoma of breast |
| HER3 | colon and gastric cancers |
| HMWMAA | melanoma, glioblastoma, breast cancer |
| IGF-I receptor | breast, prostate, lung cancers |
| IL11Rα | papillary thyroid cancer, osteosarcoma, |
| | colorectal adenocarcinoma, lymphocytic leukemia |
| IL13Rα2 | renal cell carcinoma, prostate cancer, |
| | gliomas, head and neck cancer, astrocytoma |
| KIT | myeloid leukemia, kaposi's sarcoma, |
| | erythroleukemia, gastrointestinal stromal tumors |
| KLRG2 | breast cancers, lung cancers and ovarian cancers. |
| LewisY | squamous cell lung carcinoma, lung adenocarcinoma, |
| | ovarian carcinoma, and colorectal adenocarcinoma |
| LMP2 | prostate cancer, Hodgkin's lymphoma, |
| | nasopharyngeal carcinoma |
| LRP6 | breast cancer |
| LY6K | breast, lung, ovarian, and cervical cancer |
| LYPD8 | colorectal and gastric cancers |
| Mesothelin | mesothelioma, pancreatic cancer, ovarian cancer, |
| | stomach cancer, lung cancer, endometrial cancer |
| MUC1 | breast and ovarian cancers, lung, stomach, |
| | pancreatic, prostate cancers |
| NCAM | melanoma, Wilms' tumor, small cell lung cancer, |
| | neuroblastoma, myeloma, paraganglioma, pancreatic |
| | acinar cell carcinoma, myeloid leukemia |
| NY-BR-1 | breast cancer |
| o-acetyl GD2 | neuroblastoma, melanoma |
| OR51E2 | prostate cancer |
| PANX3 | Osteosarcoma |
| PLAC1 | hepatocellular carcinoma |
| Polysialic acid | small cell lung cancer |
| PDGFR-beta | myelomonocytic leukemia, chronic myeloid |
| | leukemia, acute myelogenous leukemia, acute |
| | lymphoblastic leukemia |
| PRSS21 | colon cancer, testicular cancer, ovarian cancer |
| PSCA | prostate cancer, gastric and bladder cancers |
| PSMA | prostate cancer |
| ROR1 | metastatic cancers, chronic lymphocytic |
| | leukemia, solid tumors in lung, breast, |
| | ovarian, colon, pancreatic, sarcoma |
| SLC34A2 | bladder cancer |
| SLC39A6 | breast cancer, esophageal cancer |
| SLITRK6 | breast cancer, urothelial cancer, lung cancer |
| SSEA-4 | breast cancer, cancer stem cells, epithelial |
| | ovarian carcinoma |
| STEAP1 | prostate cancer |
| STEAP2 | prostate cancer (including castrate-resistant |
| | prostate cancer), bladder cancer, cervical cancer, |
| | lung cancer, colon cancer, kidney cancer, breast |
| | cancer, pancreatic cancer, stomach cancer, uterine |
| | cancer, ovarian cancer, preferably prostate cancer |
| TACSTD2 | carcinomas, e.g., non-small-cell lung cancer |
| TAG72 | ovarian, breast, colon, lung, pancreatic cancers, |
| | gastric cancer |
| TEM1/CD248 | colorectal cancer |
| TEM7R | colorectal cancer |
| Tn | colorectal, breast cancers, cervical, lung, |
| | stomach cancers |
| TSHR | thyroid cancer, multiple myeloma |
| Tyrosinase | prostate cancer, melanoma |
| UPK2 | bladder cancer |
| VEGFR2 | ovarian and pancreatic cancers, renal cell |
| | carcinoma, colorectal cancer, medullary thyroid |
| | carcinoma |

In another embodiment, the disease is related to autoimmunity, transplantation rejection, post-traumatic immune responses and infectious diseases (e.g., HIV). More specifically, the IL18 receptor agonists may be used in eliminating cells involved in immune cell-mediated disorders, including lymphoma; autoimmunity, transplantation rejection, graft-versus-host disease, ischemia and stroke.

A skilled artisan readily recognizes that in many cases the IL18 receptor agonists may not provide a cure but may only provide partial benefit. In some embodiments, a physiological change having some benefit is also considered therapeutically beneficial. Thus, in some embodiments, an amount of IL18 receptor agonist that provides a physiological change is considered an “effective amount” or a “therapeutically effective amount”.

The subject, patient, or individual in need of treatment is typically a mammal, more specifically a human.

For the prevention or treatment of disease, the appropriate dosage of an IL18 receptor agonist of the disclosure (when used alone or in combination with one or more other additional therapeutic agents) will depend on the type of disease to be treated, the route of administration, the body weight of the patient, the particular IL18 receptor agonist, the severity and course of the disease, whether the antibody is administered for preventive or therapeutic purposes, previous or concurrent therapeutic interventions, the patient's clinical history and response to the IL18 receptor agonist, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A single administration of unconjugated IL18 can range from about 50,000 IU/kg to about 1,000,000 IU/kg or more, more typically about 600,000 IU/kg of IL18. This may be repeated several times a day (e.g., 2-3 times.), for several days (e.g., about 3-5 consecutive days) and then may be repeated one or more times following a period of rest (e.g., about 7-14 days). Thus, a therapeutically effective amount may comprise only a single administration or many administrations over a period of time (e.g., about 20-30 individual administrations of about 600,000 IU/kg of IL18 each given over about a 10-20 day period).

Similarly, the IL18 receptor agonist is suitably administered to the patient at one time or over a series of treatments. Depending on the type and severity of the disease, about 1 μg/kg to 15 mg/kg (e.g., 0.1 mg/kg-10 mg/kg) of IL18 receptor agonist can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the IL18 receptor agonist would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the IL18 receptor agonist). An initial higher loading dose, followed by one or more lower doses may be administered. However, other dosage regimens may be useful. The progress of this therapy is easily monitored by conventional techniques and assays.

The IL18 receptor agonists of the disclosure will generally be used in an amount effective to achieve the intended purpose. For use to treat or prevent a disease condition, the IL18 receptor agonists of the disclosure, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the IL18 receptor agonists which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the IL18 receptor agonists may not be related to plasma concentration. One having skill in the art will be able to optimize therapeutically effective local dosages without undue experimentation.

A therapeutically effective dose of the IL18 receptor agonists described herein will generally provide therapeutic benefit without causing substantial toxicity. Toxicity and therapeutic efficacy of an IL18 receptor agonist can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. IL18 receptor agonists that exhibit large therapeutic indices are preferred. In one embodiment, the IL18 receptor agonist according to the present disclosure exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with IL18 receptor agonists of the disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Due to lower toxicity, the IL18 receptor agonists of the disclosure can have higher maximum therapeutic doses than wild type IL18.

### 6.10.1. Combination Therapy

The IL18 receptor agonists according to the disclosure may be administered in combination with one or more other agents in therapy. For instance, an IL18 receptor agonist of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of IL18 receptor agonist used, the type of disorder or treatment, and other factors discussed above. The IL18 receptor agonists are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the IL18 receptor agonist of the disclosure can occur prior to, simultaneously, and/or following, administration of the additional therapeutic agent and/or adjuvant. IL18 receptor agonists of the disclosure can also be used in combination with radiation therapy.

The IL18 receptor agonists of the disclosure can be advantageously used in combination with immune checkpoint blockade therapy. Accordingly, contemplated herein, in some embodiments, is a method of treating cancer comprising administering to a subject: (1) an IL18 receptor agonist of the disclosure comprising a targeting domain that binds to an immune checkpoint molecule, and (2) an antibody or antigen-binding molecule that specifically binds to an immune checkpoint molecule (e.g., CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2). In some embodiments, disclosed is a method for treating cancer comprising administering, to a subject in need thereof, (1) an IL18 receptor agonist of the disclosure comprising a targeting domain that binds to an immune checkpoint molecule, and (2) and anti-PD1 antibody. In some embodiments, disclosed is a method for treating cancer comprising administering, to a subject in need thereof, (1) an IL18 receptor agonist of the disclosure comprising a targeting domain that binds to an immune checkpoint molecule, and (2) and anti-PDL1 antibody.

### 6.11. Sequences

Certain sequences of the disclosure are provided in Table S below.

TABLE S
Sequences

| | | SEQ ID |
|---|---|---|
| Name | Sequence | NO: |
| Full-length | MAAEPVEDNCINFVAMKFIDNTLYFIAEDDENLESDYFGKLESK | 1 |
| human IL18 | LSVIRNLNDQVLFIDQGNRPLFEDMTDSDCRDNAPRTIFIISMYK | |
| | DSQPRGMAVTISVKCEKISTLSCENKIISFKEMNPPDNIKDTKSD | |
| | IIFFQRSVPGHDNKMQFESSSYEGYFLACEKERDLFKLILKKED | |
| | ELGDRSIMFTVQNED | |
| Mature | YFGKLESKLSVIRNLNDQVLFIDQGNRPLFEDMTDSDCRDNAP | 2 |
| human IL18 | RTIFIISMYKDSQPRGMAVTISVKCEKISTLSCENKIISFKEMNPP | |
| | DNIKDTKSDIIFFQRSVPGHDNKMQFESSSYEGYFLACEKERD | |
| | LFKLILKKEDELGDRSIMFTVQNED | |
| Full-length | MAAMSEDSCVNFKEMMFIDNTLYFIPEENGDLESDNFGRLHCT | 3 |
| murine IL18 | TAVIRNINDQVLFVDKRQPVFEDMTDIDQSASEPQTRLIIYMYK | |
| | DSEVRGLAVTLSVKDSKMSTLSCKNKIISFEEMDPPENIDDIQS | |
| | DLIFFQKRVPGHNKMEFESSLYEGHFLACQKEDDAFKLILKKKD | |
| | ENGDKSVMFTLTNLHQS | |
| Mature | NFGRLHCTTAVIRNINDQVLFVDKRQPVFEDMTDIDQSASEPQ | 4 |
| murine IL18 | TRLIIYMYKDSEVRGLAVTLSVKDSKMSTLSCKNKIISFEEMDPP | |
| | ENIDDIQSDLIFFQKRVPGHNKMEFESSLYEGHFLACQKEDDA | |
| | FKLILKKKDENGDKSVMFTLTNLHQS | |
| Mature | AESCTSRPHITVVEGEPFYLKHCSCSLAHEIETTTKSWYKSSG | 5 |
| human | SQEHVELNPRSSSRIALHDCVLEFWPVELNDTGSYFFQMKNY | |
| IL18Rα | TQKWKLNVIRRNKHSCFTERQVTSKIVEVKKFFQITCENSYYQT | |
| Extracellular | LVNSTSLYKNCKKLLLENNKNPTIKKNAEFEDQGYYSCVHFLH | |
| domain | HNGKLFNITKTFNITIVEDRSNIVPVLLGPKLNHVAVELGKNVRL | |
| | NCSALLNEEDVIYWMFGEENGSDPNIHEEKEMRIMTPEGKWH | |
| | ASKVLRIENIGESNLNVLYNCTVASTGGTDTKSFILVRKADMADI | |
| | PGHVFTR | |
| Human | AESCTSRPHITWEGEPFYLKHCSCSLAHEIETTTKSWYKSSG | 6 |
| IL18Rα D1 | SQEHVELNPRSSSRIALHDCVLEFWPVELNDTGSYFFQMKNY | |
| | TQKWKLNVIRR | |
| Human | NKHSCFTERQVTSKIVEVKKFFQITCENSYYQTLVNSTSLYKNC | 7 |
| IL18Rα D2 | KKLLLENNKNPTIKKNAEFEDQGYYSCVHFLHHNGKLFNITKTF | |
| | NITIVED | |
| Human | RSNIVPVLLGPKLNHVAVELGKNVRLNCSALLNEEDVIYWMFG | 8 |
| IL18Rα D3 | EENGSDPNIHEEKEMRIMTPEGKWHASKVLRIENIGESNLNVL | |
| | YNCTVASTGGTDTKSFILVRK | |
| Human | AESCTSRPHITWEGEPFYLKHCSCSLAHEIETTTKSWYKSSG | 9 |
| IL18Rα D1- | SQEHVELNPRSSSRIALHDCVLEFWPVELNDTGSYFFQMKNY | |
| D3 | TQKWKLNVIRRNKHSCFTERQVTSKIVEVKKFFQITCENSYYQT | |
| | LVNSTSLYKNCKKLLLENNKNPTIKKNAEFEDQGYYSCVHFLH | |
| | HNGKLFNITKTFNITIVEDRSNIVPVLLGPKLNHVAVELGKNVRL | |
| | NCSALLNEEDVIYWMFGEENGSDPNIHEEKEMRIMTPEGKWH | |
| | ASKVLRIENIGESNLNVLYNCTVASTGGTDTKSFILVRK | |
| Human | KKFFQITCENSYYQTLVNSTSLYKNCKKLLLENNKNPTIKKNAE | 10 |
| IL18Rα D2- | FEDQGYYSCVHFLHHNGKLFNITKTFNITIVEDRSNIVPVLLGPK | |
| D3 | LNHVAVELGKNVRLNCSALLNEEDVIYWMFGEENGSDPNIHEE | |
| | KEMRIMTPEGKWHASKVLRIENIGESNLNVLYNCTVASTGGTD | |
| | TKSFILVRK | |
| Mature | KSCIHRSQIHVVEGEPFYLKPCGISAPVHRNETATMRWFKGSA | 11 |
| murine | SHEYRELNNRSSPRVTFHDHTLEFWPVEMEDEGTYISQVGND | |
| IL18Rα | RRNWTLNVTKRNKHSCFSDKLVTSRDVEVNKSLHITCKNPNYE | |
| Extracellular | ELIQDTWLYKNCKEISKTPRILKDAEFGDEGYYSCVFSVHHNGT | |
| domain | RYNITKTVNITVIEGRSKVTPAILGPKCEKVGVELGKDVELNCSA | |
| | SLNKDDLFYWSIRKEDSSDPNVQEDRKETTTWISEGKLHASKIL | |
| | RFQKITENYLNVLYNCTVANEEAIDTKSFVLVRKEIPDIPGHVFT | |
| | G | |
| hIgG1 Fc | EPKSCDKTHTCPPCPAPELLGGPSVFLFPPKPKDTLMISRTPE | 12 |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPGK | |
| hIgG2 Fc | ERKCCVECPPCPAPPVAGPSVFLFPPKPKDTLMISRTPEVTCV | 13 |
| | WVDVSHEDPEVQFNWYVDGVEVHNAKTKPREEQFNSTFRVV | |
| | SVLTWHQDWLNGKEYKCKVSNKGLPAPIEKTISKTKGQPREP | |
| | QVYTLPPSREEMTKNQVSLTCLVKGFYPSDISVEWESNGQPE | |
| | NNYKTTPPMLDSDGSFFLYSKLTVDKSRWQQGNVFSCSVMH | |
| | EALHNHYTQKSLSLSPGK | |
| hIgG3 Fc | ELKTPLGDTTHTCPRCPEPKSCDTPPPCPRCPEPKSCDTPPP | 14 |
| | CPRCPEPKSCDTPPPCPRCPAPELLGGPSVFLFPPKPKDTLMI | |
| | SRTPEVTCVVVDVSHEDPEVQFKWYVDGVEVHNAKTKPREE | |
| | QYNSTFRVVSVLTVLHQDWLNGKEYKCKVSNKALPAPIEKTIS | |
| | KTKGQPREPQVYTLPPSREEMTKNQVSLTCLVKGFYPSDIAVE | |
| | WESSGQPENNYNTTPPMLDSDGSFFLYSKLTVDKSRWQQGNI | |
| | FSCSVMHEALHNRFTQKSLSLSPGK | |
| hIgG4 Fc | ESKYGPPCPSCPAPEFLGGPSVFLFPPKPKDTLMISRTPEVTC | 15 |
| | VVVDVSQEDPEVQFNWYVDGVEVHNAKTKPREEQFNSTYRV | |
| | VSVLTVLHQDWLNGKEYKCKVSNKGLPSSIEKTISKAKGQPRE | |
| | PQVYTLPPSQEEMTKNQVSLTCLVKGFYPSDIAVEWESNGQP | |
| | ENNYKTTPPVLDSDGSFFLYSRLTVDKSRWQEGNVFSCSVMH | |
| | EALHNHYTQKSLSLSLGK | |
| hIgG4s Fc | ESKYGPPCPPCPAPPVAGPSVFLFPPKPKDTLMISRTPEVTCV | 16 |
| | WVDVSQEDPEVQFNWYVDGVEVHNAKTKPREEQFNSTYRVV | |
| | SVLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMHE | |
| | ALHNHYTQKSLSLSLGK | |
| hIgG1 PVA | EPKSCDKTHTCPPCPAPPVAGPSVFLFPPKPKDTLMISRTPEV | 17 |
| Fc | TCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPGK | |
| hIgG1 PVA | EPKSCDKTHTCPPCPAPPVAGPSVFLFPPKPKDTLMISRTPEV | 18 |
| star: | TCVVVDVSHEDPEVKFNWYVDGVEVHNAKTKPREEQYNSTY | |
| | RVVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNRFTQKSLSLSPGK | |
| hIgG1s: | DKKVEPKSCDKTHTCPPCPAPPVAGPSVFLFPPKPKDTLMISR | 19 |
| | TPEVTCVVVDVSQEDPEVQFNWYVDGVEVHNAKTKPREEQF | |
| | NSTYRVVSVLTVLHQDWLNGKEYKCKVSNKGLPSSIEKTISKA | |
| | KGQPREPQVYTLPPSRDELTKNQVSLTCLVKGFYPSDIAVEWE | |
| | SNGQPENNYKTTPPVLDSDGSFFLYSKLTVDKSRWQQGNVFS | |
| | CSVMHEALHNHYTQKSLSLSPGK | |
| hIgG1 | EPKSCDKTHTCPPCPAPELLGGPSVFLFPPKPKDTLMISRTPE | 20 |
| N180G, | VTCVVVDVSHEDPEVKFNWYVDGVEVHNAKTKPREEQYGST | |
| also | YRVVSVLTVLHQDWLNGKEYKCKVSNKALPAPIEKTISKAKGQ | |
| referred to | PREPQVYTLPPSRDELTKNQVSLTCLVKGFYPSDIAVEWESNG | |
| as N297G: | QPENNYKTTPPVLDSDGSFFLYSKLTVDKSRWQQGNVFSCSV | |
| | MHEALHNHYTQKSLSLSPGK | |
| hIgG2 | ERKCCVECPPCPAPPVAGPSVFLFPPKPKDTLMISRTPEVTCV | 21 |
| variant | WVDVSHEDPEVQFNWYVDGVEVHNAKTKPREEQFNSTFRVV | |
| | SVLTWHQDWLNGKEYKCKVSNKGLPAPIEKTISKTKGQPREP | |
| | QVYTLPPSREEMTKNQVSLTCLVKGFYPSDIAVEWESNGQPE | |
| | NNYKTTPPMLDSDGSFFLYSKLTVDKSRWQQGNVFSCSVMH | |
| | EALHNHYTQKSLSLSPGK | |
| IgG1PVA | EPKSCDKTHTCPPCPAPPVAGPSVFLFPPKPKDTLMISRTPEV | 22 |
| hinge-Fc | TCVVVDVSHEDPEVKFNWYVDGVEVHNAKTKPREEQYNSTY | |
| knob | RVVSVLTVLHQDWLNGKEYKCKVSNKALPAPIEKTISKAKGQP | |
| | REPQVYTLPPSRDELTKNQVSLWCLVKGFYPSDIAVEWESNG | |
| | QPENNYKTTPPVLDSDGSFFLYSKLTVDKSRWQQGNVFSCSV | |
| | MHEALHNHYTQKSLSLSPGK | |
| IgG1PVA | EPKSCDKTHTCPPCPAPPVAGPSVFLFPPKPKDTLMISRTPEV | 23 |
| hinge-Fc | TCVVVDVSHEDPEVKFNWYVDGVEVHNAKTKPREEQYNSTY | |
| knob star | RVVSVLTVLHQDWLNGKEYKCKVSNKALPAPIEKTISKAKGQP | |
| | REPQVYTLPPSRDELTKNQVSLWCLVKGFYPSDIAVEWESNG | |
| | QPENNYKTTPPVLDSDGSFFLYSKLTVDKSRWQQGNVFSCSV | |
| | MHEALHNRFTQKSLSLSPGK | |
| IgG1PVA | EPKSCDKTHTCPPCPAPPVAGPSVFLFPPKPKDTLMISRTPEV | 24 |
| hinge-Fc | TCVVVDVSHEDPEVKFNWYVDGVEVHNAKTKPREEQYNSTY | |
| knob Cys | RVVSVLTVLHQDWLNGKEYKCKVSNKALPAPIEKTISKAKGQP | |
| | REPQVYTLPPCRDELTKNQVSLWCLVKGFYPSDIAVEWESNG | |
| | QPENNYKTTPPVLDSDGSFFLYSKLTVDKSRWQQGNVFSCSV | |
| | MHEALHNHYTQKSLSLSPGK | |
| IgG1PVA | EPKSCDKTHTCPPCPAPPVAGPSVFLFPPKPKDTLMISRTPEV | 25 |
| hinge-Fc | TCVVVDVSHEDPEVKFNWYVDGVEVHNAKTKPREEQYNSTY | |
| knob Cys | RVVSVLTVLHQDWLNGKEYKCKVSNKALPAPIEKTISKAKGQP | |
| star | REPQVYTLPPCRDELTKNQVSLWCLVKGFYPSDIAVEWESNG | |
| | QPENNYKTTPPVLDSDGSFFLYSKLTVDKSRWQQGNVFSCSV | |
| | MHEALHNRFTQKSLSLSPGK | |
| IgG1PVA | EPKSCDKTHTCPPCPAPPVAGPSVFLFPPKPKDTLMISRTPEV | 26 |
| hinge-Fc | TCVVVDVSHEDPEVKFNWYVDGVEVHNAKTKPREEQYNSTY | |
| Hole | RVVSVLTVLHQDWLNGKEYKCKVSNKALPAPIEKTISKAKGQP | |
| | REPQVYTLPPSRDELTKNQVSLSCAVKGFYPSDIAVEWESNG | |
| | QPENNYKTTPPVLDSDGSFFLVSKLTVDKSRWQQGNVFSCSV | |
| | MHEALHNHYTQKSLSLSPGK | |
| IgG1PVA | EPKSCDKTHTCPPCPAPPVAGPSVFLFPPKPKDTLMISRTPEV | 27 |
| hinge-Fc | TCVVVDVSHEDPEVKFNWYVDGVEVHNAKTKPREEQYNSTY | |
| Hole star | RVVSVLTVLHQDWLNGKEYKCKVSNKALPAPIEKTISKAKGQP | |
| | REPQVYTLPPSRDELTKNQVSLSCAVKGFYPSDIAVEWESNG | |
| | QPENNYKTTPPVLDSDGSFFLVSKLTVDKSRWQQGNVFSCSV | |
| | MHEALHNRFTQKSLSLSPGK | |
| IgG1PVA | EPKSCDKTHTCPPCPAPPVAGPSVFLFPPKPKDTLMISRTPEV | 28 |
| hinge-Fc | TCVVVDVSHEDPEVKFNWYVDGVEVHNAKTKPREEQYNSTY | |
| Hole Cys | RVVSVLTVLHQDWLNGKEYKCKVSNKALPAPIEKTISKAKGQP | |
| | REPQVCTLPPSRDELTKNQVSLSCAVKGFYPSDIAVEWESNG | |
| | QPENNYKTTPPVLDSDGSFFLVSKLTVDKSRWQQGNVFSCSV | |
| | MHEALHNHYTQKSLSLSPGK | |
| IgG1PVA | EPKSCDKTHTCPPCPAPPVAGPSVFLFPPKPKDTLMISRTPEV | 29 |
| hinge-Fc | TCVVVDVSHEDPEVKFNWYVDGVEVHNAKTKPREEQYNSTY | |
| Hole Cys | RVVSVLTVLHQDWLNGKEYKCKVSNKALPAPIEKTISKAKGQP | |
| star | REPQVCTLPPSRDELTKNQVSLSCAVKGFYPSDIAVEWESNG | |
| | QPENNYKTTPPVLDSDGSFFLVSKLTVDKSRWQQGNVFSCSV | |
| | MHEALHNRFTQKSLSLSPGK | |
| hIgG4 | ESKYGPPCPPCPAPEFLGGPSVFLFPPKPKDTLMISRTPEVTC | 30 |
| S108P | WWVDVSQEDPEVQFNWYVDGVEVHNAKTKPREEQFNSTYRV | |
| | VSVLTVLHQDWLNGKEYKCKVSNKGLPSSIEKTISKAKGQPRE | |
| | PQVYTLPPSQEEMTKNQVSLTCLVKGFYPSDIAVEWESNGQP | |
| | ENNYKTTPPVLDSDGSFFLYSRLTVDKSRWQEGNVFSCSVMH | |
| | EALHNHYTQKSLSLSLGK | |
| hIgG1 | EPKSCDKTHTCPPCPAPPVAGPSVFLFPPKPKDTLMISRTPEVTCVVVDVSH | 134 |
| PVA/P329A | EDPEVKFNWYVDGVEVHNAKTKPREEQYNSTYRVVSVLTVLHQDWLNGKEYK | |
| Fc | CKVSNKALAAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVFS | |
| | CSVMHEALHNHYTQKSLSLSPGK | |
| hIgG1 | EPKSCDKTHTCPPCPAPEAAGGPSVFLFPPKPKDTLMISRTPEVTCVVVDVS | 135 |
| LALAPG Fc | HEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLTCLVKG | |
| | FYPSDIAVEWESNGQPENNYKTTPPVLDSDGSFFLYSKLTVDKSRWQQGNVF | |
| | SCSVMHEALHNHYTQKSLSLSPGK | |
| hIgG1us | DKKVEPKSCDKTHTCPPCPAPGGGGPSVFLFPPKPKDTLMISRTPEVTCVVV | 136 |
| | DVSQEDPEVQFNWYVDGVEVHNAKTKPREEQFNSTYRVVSVLTVLHQDWLNG | |
| | KEYKCKVSNKGLPSSIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. An IL18 receptor agonist comprising, on a first polypeptide chain and a second polypeptide chain dimerized through a first Fc domain and a second Fc domain:
(a) an IL18 moiety N-terminal to the first Fc domain or the second Fc domain; and
(b) an IL18Rα moiety N-terminal to the first Fc domain or the second Fc domain.

2. The IL18 receptor agonist of embodiment 1, which comprises an IL18 moiety and an IL18Rα moiety on the same polypeptide chain.

3. The IL18 receptor agonist of embodiment 1, which comprises an IL18 moiety and an IL18Rα moiety on different polypeptide chains.

4. The IL18 receptor agonist of any one of embodiments 1 to 3, wherein the IL18 moiety is N-terminal to the first Fc domain.

5. The IL18 receptor agonist of any one of embodiments 1 to 3, wherein the IL18 moiety is N-terminal to the second Fc domain.

6. The IL18 receptor agonist of any one of embodiments 1 to 5, wherein the IL18Rα moiety is N-terminal to the first Fc domain.

7. The IL18 receptor agonist of any one of embodiments 1 to 5, wherein the IL18Rα moiety is N-terminal to the second Fc domain.

8. The IL18 receptor agonist of any one of embodiments 1 to 7, which is monovalent for the IL18 moiety.

9. The IL18 receptor agonist of any one of embodiments 1 to 7, which is bivalent for the IL18 moiety.

10. The IL18 receptor agonist of any one of embodiments 1 to 9, which is monovalent for the IL18Rα moiety.

11. The IL18 receptor agonist of any one of embodiments 1 to 9, which is bivalent for the IL18Rα moiety.

12. The IL18 receptor agonist of any one of embodiments 1 to 11, which lacks an IL18 moiety C-terminal to the first Fc domain or second Fc domain.

13. The IL18 receptor agonist of any one of embodiments 1 to 11, which lacks an IL18Rα moiety C-terminal to the first Fc domain or second Fc domain.

14. The IL18 receptor agonist of any one of embodiments 1 to 13, wherein the IL18Rα moiety is configured to mask the IL18 moiety.

15. The IL18 receptor agonist of any one of embodiments 1 to 14, further comprising a targeting moiety.

16. An IL18 receptor agonist, which is optionally an IL18 receptor agonist according to any one of embodiments 1 to 15, comprising:
(a) a first polypeptide chain comprising, in N- to C-terminal orientation, an IL18 moiety and a first Fc domain; and
(b) a second polypeptide chain comprising, in N- to C-terminal orientation, an IL18Rα moiety and a second Fc domain.

17. The IL18 receptor agonist of embodiment 16, wherein the first polypeptide lacks an IL18Rα moiety.

18. The IL18 receptor agonist of embodiment 16, wherein the second polypeptide lacks an IL18 moiety.

19. The IL18 receptor agonist of any one of embodiments 16 to 18, which is monovalent for the IL18 moiety.

20. The IL18 receptor agonist of any one of embodiments 16 to 19, which is monovalent for the IL18Rα moiety.

21. The IL18 receptor agonist of any one of embodiments 16 to 20, wherein the IL18 moiety is connected to the first Fc domain via a linker.

22. The IL18 receptor agonist of embodiment 21, wherein the linker comprises or consists of any sequence set forth in Table L.

23. The IL18 receptor agonist of any one of embodiments 16 to 22, wherein the IL18Rα moiety is connected to the second Fc domain via a linker.

24. The IL18 receptor agonist of embodiment 23, wherein the linker comprises or consists of any sequence set forth in Table L.

25. An IL18 receptor agonist, optionally an IL18 receptor agonist according to any one of embodiments 16 to 24, having a configuration as depicted in FIG. 1D.

26. The IL18 receptor agonist of any one of embodiments 16 to 25, further comprising a targeting moiety.

27. The IL18 receptor agonist of embodiment 26, wherein the targeting moiety or a component thereof is C-terminal to the first Fc domain.

28. The IL18 receptor agonist of embodiment 27, wherein the targeting moiety is a Fab.

29. The IL18 receptor agonist of embodiment 28, wherein the first polypeptide comprises, in N- to C-terminal orientation, the IL18 moiety, the first Fc domain, a first VH domain, and a first CH1 domain.

30. The IL18 receptor agonist of embodiment 29, further comprising a third polypeptide comprising a first VL domain and a first CL domain associated with the first VH domain and first CH1 domain, respectively.

31. The IL18 receptor agonist of embodiment 30, wherein the third polypeptide is a universal light chain.

32. The IL18 receptor agonist of any one of embodiments 26 to 31, further comprising an additional targeting moiety.

33. The IL18 receptor agonist of embodiment 32, wherein the additional targeting moiety or a component thereof is C-terminal to the second Fc domain.

34. The IL18 receptor agonist of embodiment 33, wherein the additional targeting moiety is a Fab.

35. The IL18 receptor agonist of embodiment 34, wherein the second polypeptide comprises, in N- to C-terminal orientation, the IL18Rα moiety, the second Fc domain, a second VH domain, and a second CH1 domain.

36. The IL18 receptor agonist of embodiment 35, further comprising a fourth polypeptide comprising a second VL domain and a second CL domain associated with the second VH domain and second CH1 domain, respectively.

37. The IL18 receptor agonist of embodiment 36, wherein the fourth polypeptide is a universal light chain.

38. The IL18 receptor agonist of any one of embodiments 35 to 37, wherein the first VH domain and the second VH domain are identical.

39. The IL18 receptor agonist of any one of embodiments 35 to 37, wherein the first VH domain and the second VH domain are non-identical.

40. The IL18 receptor agonist of embodiment 27, wherein the targeting moiety is an scFv.

41. The IL18 receptor agonist of embodiment 32, wherein the additional targeting moiety is an scFv.

42. An IL18 receptor agonist, optionally an IL18 receptor agonist according to any one of embodiments 26 to 41, having a configuration as depicted in FIG. 2C.

43. An IL18 receptor agonist, which is optionally an IL18 receptor agonist according to any one of embodiments 1 to 15, comprising:
(a) a first polypeptide chain comprising, in N- to C-terminal orientation, an IL18 moiety, an IL18Rα moiety, and a first Fc domain; and
(b) a second polypeptide chain comprising a second Fc domain.

44. The IL18 receptor agonist of embodiment 43, wherein the IL18 moiety and the IL18Rα moiety are connected via a linker.

45. The IL18 receptor agonist of embodiment 44, wherein the linker comprises or consists of any sequence set forth in Table L.

46. The IL18 receptor agonist of any one of embodiments 43 to 45, wherein the IL18Rα moiety is connected to the first Fc domain via a linker.

47. The IL18 receptor agonist of embodiment 46, wherein the linker comprises or consists of any sequence set forth in Table L.

48. The IL18 receptor agonist of any one of embodiments 43 to 47, which is monovalent for the IL18 moiety.

49. The IL18 receptor agonist of any one of embodiments 43 to 47, which is bivalent for the IL18 moiety.

50. The IL18 receptor agonist of any one of embodiments 43 to 49, which is monovalent for the IL18Rα moiety.

51. The IL18 receptor agonist of any one of embodiments 43 to 49, which is bivalent for the IL18Rα moiety.

52. The IL18 receptor agonist of any one of embodiments 43 to 47, wherein the second polypeptide lacks an IL18 moiety.

53. The IL18 receptor agonist of any one of embodiments 43 to 47 or 52, wherein the second polypeptide lacks an IL18Rα moiety.

54. The IL18 receptor agonist of any one of embodiments 43 to 47, wherein the second polypeptide further comprises, N-terminal to the second Fc domain, an additional IL18 moiety and an additional IL18Rα moiety.

55. The IL18 receptor agonist of embodiment 54, wherein the additional IL18 moiety is N-terminal to the additional IL18Rα moiety.

56. The IL18 receptor agonist of embodiment 54 or 55, wherein the additional IL18 moiety and the additional IL18Rα moiety are connected via a linker.

57. The linker of embodiment 56, wherein the linker comprises or consists of any sequence set forth in Table L.

58. An IL18 receptor agonist, optionally an IL18 receptor agonist according any one of embodiments 43 to 57, having a configuration as depicted in FIG. 1B.

59. An IL18 receptor agonist, optionally an IL18 receptor agonist according any one of embodiments 43 to 57, having a configuration as depicted in FIG. 1C.

60. The IL18 receptor agonist of any one of embodiments 43 to 59, further comprising a targeting moiety.

61. The IL18 receptor agonist of embodiment 60, wherein the targeting moiety or a component thereof is N-terminal to the second Fc domain.

62. The IL18 receptor agonist of embodiment 60 or 61, wherein the targeting moiety is a Fab.

63. The IL18 receptor agonist of embodiment 62, wherein the second polypeptide comprises, in N- to C-terminal orientation, a VH domain, a CH1 domain, and the second Fc domain.

64. The IL18 receptor agonist of embodiment 63, further comprising a third polypeptide comprising, in N- to C-terminal orientation, a VL domain and a CL domain.

65. The IL18 receptor agonist of embodiment 64, wherein the third polypeptide is a universal light chain.

66. An IL18 receptor agonist, optionally an IL18 receptor agonist according to any one of embodiments 60 to 65, having a configuration as depicted in FIG. 2B.

67. The IL18 receptor agonist of any one of embodiments 15, 26 to 42, and 60 to 66, wherein the targeting moiety is capable of binding to a target molecule identified in Section 6.5.

68. The IL18 receptor agonist of any one of embodiments 15, 26 to 42, and 60 to 67, wherein the targeting moiety (a) comprises the (i) CDR or (ii) VH and VL sequences of antibody set forth in Table A or (b) competes with the antibody set forth in Table A for binding to the target molecule.

69. The IL18 receptor agonist of any one of embodiments 15, 26 to 42, and 60 to 66, wherein the targeting moiety comprises means for binding to a target molecule identified in Section 6.5.

70. The IL18 receptor agonist of embodiment 69, wherein the targeting moiety comprises means for binding to PDL1, e.g., as described in Table A-1.

71. The IL18 receptor agonist of embodiment 69, wherein the targeting moiety comprises means for binding to PD1, e.g., as described in Table A-2.

72. The IL18 receptor agonist of any one of embodiments 15, 26 to 42, and 60 to 66, wherein the targeting moiety is a tumor antigen targeting moiety.

73. The IL18 receptor agonist of embodiment 72, wherein the tumor antigen targeting moiety is capable of binding to a tumor-associated antigen (TAA).

74. The IL18 receptor agonist of embodiment 73, wherein the TAA is CD20, EGFR, FITC, CD19, CD22, CD33, PSMA, GD2, EGFR variants, ROR1, c-Met, HER2, CEA, mesothelin, GM2, CD7, CD10, CD30, CD34, CD38, CD41, CD44, CD74, CD123 CD133, CD171, MUC16, MUC1, CS1 (CD319), IL-13Ra2, BCMA, Lewis Y, IgG kappa chain, folate receptor-alpha, PSCA, or EpCAM.

75. The IL18 receptor agonist of embodiment 72, wherein the targeting moiety is capable of binding to an ECM antigen.

76. The IL18 receptor agonist of embodiment 75, wherein the ECM antigen is syndecan, heparanase, integrins, osteopontin, link, cadherins, laminin, laminin type EGF, lectin, fibronectin, notch, nectin (e.g., nectin-4), tenascin, collagen (e.g., collagen type X) or matrixin.

77. The IL18 receptor agonist of embodiment 72, wherein the targeting moiety is capable of binding to a cell surface molecule of tumor or viral lymphocytes.

78. The IL18 receptor agonist of embodiment 77, wherein the cell surface molecule is a T-cell co-stimulatory protein.

79. The IL18 receptor agonist of embodiment 78, wherein the T-cell co-stimulatory protein is CD27, CD28, 4-1BB (CD137), OX40, CD30, CD40, ICOS, lymphocyte function-associated antigen-1 (LFA-1), CD2, CD7, LIGHT, NKG2C, or B7-H3.

80. The IL18 receptor agonist of embodiment 72, wherein the targeting moiety is capable of binding to an immune checkpoint molecule.

81. The IL18 receptor agonist of embodiment 80, wherein the immune checkpoint molecule is CTLA-4, PD1, PDL1, PDL2, B7-H3, B7-H4, BTLA, HVEM, TIM3, GAL9, LAG3, VISTA, KIR, 2B4, CD160, CGEN-15049, CHK1, or CHK2.

82. The IL18 receptor agonist of embodiment 81, wherein the immune checkpoint molecule is PD1.

83. The IL18 receptor agonist of embodiment 81, wherein the immune checkpoint molecule is human PD1.

84. The IL18 receptor agonist of embodiment 81, wherein the immune checkpoint molecule is PDL1.

85. The IL18 receptor agonist of embodiment 81, wherein the immune checkpoint molecule is human PDL1.

86. The IL18 receptor agonist of any one of embodiments 1 to 85, wherein the IL18Rα moiety comprises D3 of IL18Rα.

87. The IL18 receptor agonist of any one of embodiments 1 to 85, wherein the IL18Rα moiety comprises D2 and D3 of IL18Rα.

88. The IL18 receptor agonist of any one of embodiments 1 to 85, wherein the IL18Rα moiety comprises D1, D2, and D3 of IL18Rα.

89. The IL18 receptor agonist of any one of embodiments 1 to 85, wherein the IL18Rα moiety lacks D1 of IL18Rα.

90. The IL18 receptor agonist of any one of embodiments 1 to 85, wherein the IL18Rα moiety lacks D2 of IL18Rα.

91. The IL18 receptor agonist of any one of embodiments 86 to 90, wherein the IL18Rα is human IL18Rα.

92. The IL18 receptor agonist of any one of embodiments 86 to 90, wherein the IL18Rα is murine IL18Rα.

93. The IL18 receptor agonist of any one of embodiments 1 to 85, wherein the IL18Rα moiety comprises or consists of an amino acid sequence having at least 90% sequence identity to the amino acid sequence of SEQ ID NO:8.

94. The IL18 receptor agonist of any one of embodiments 1 to 85, wherein the IL18Rα moiety comprises or consists of an amino acid sequence having at least 91% sequence identity to the amino acid sequence of SEQ ID NO:8.

95. The IL18 receptor agonist of any one of embodiments 1 to 85, wherein the IL18Rα moiety comprises or consists of an amino acid sequence having at least 92% sequence identity to the amino acid sequence of SEQ ID NO:8.

96. The IL18 receptor agonist of any one of embodiments 1 to 85, wherein the IL18Rα moiety comprises or consists of an amino acid sequence having at least 93% sequence identity to the amino acid sequence of SEQ ID NO:8.

97. The IL18 receptor agonist of any one of embodiments 1 to 85, wherein the IL18Rα moiety comprises or consists of an amino acid sequence having at least 94% sequence identity to the amino acid sequence of SEQ ID NO:8.

98. The IL18 receptor agonist of any one of embodiments 1 to 85, wherein the IL18Rα moiety comprises or consists of an amino acid sequence having at least 95% sequence identity to the amino acid sequence of SEQ ID NO:8.

99. The IL18 receptor agonist of any one of embodiments 1 to 85, wherein the IL18Rα moiety comprises or consists of an amino acid sequence having at least 96% sequence identity to the amino acid sequence of SEQ ID NO:8.

100. The IL18 receptor agonist of any one of embodiments 1 to 85, wherein the IL18Rα moiety comprises or consists of an amino acid sequence having at least 97% sequence identity to the amino acid sequence of SEQ ID NO:8.

101. The IL18 receptor agonist of any one of embodiments 1 to 85, wherein the IL18Rα moiety comprises or consists of an amino acid sequence having at least 98% sequence identity to the amino acid sequence of SEQ ID NO:8.

102. The IL18 receptor agonist of any one of embodiments 1 to 85, wherein the IL18Rα moiety comprises or consists of an amino acid sequence having at least 99% sequence identity to the amino acid sequence of SEQ ID NO:8.

103. The IL18 receptor agonist of any one of embodiments 1 to 85, wherein the IL18Rα moiety comprises or consists of the amino acid sequence of SEQ ID NO:8.

104. The IL18 receptor agonist of any one of embodiments 1 to 103, wherein the IL18Rα moiety comprises or consists of an amino acid sequence having at least 90% sequence identity to the amino acid sequence of SEQ ID NO:10.

105. The IL18 receptor agonist of any one of embodiments 1 to 103, wherein the IL18Rα moiety comprises or consists of an amino acid sequence having at least 91% sequence identity to the amino acid sequence of SEQ ID NO:10.

106. The IL18 receptor agonist of any one of embodiments 1 to 103, wherein the IL18Rα moiety comprises or consists of an amino acid sequence having at least 92% sequence identity to the amino acid sequence of SEQ ID NO:10.

107. The IL18 receptor agonist of any one of embodiments 1 to 103, wherein the IL18Rα moiety comprises or consists of an amino acid sequence having at least 93% sequence identity to the amino acid sequence of SEQ ID NO:10.

108. The IL18 receptor agonist of any one of embodiments 1 to 103, wherein the IL18Rα moiety comprises or consists of an amino acid sequence having at least 94% sequence identity to the amino acid sequence of SEQ ID NO:10.

109. The IL18 receptor agonist of any one of embodiments 1 to 103, wherein the IL18Rα moiety comprises or consists of an amino acid sequence having at least 95% sequence identity to the amino acid sequence of SEQ ID NO:10.

110. The IL18 receptor agonist of any one of embodiments 1 to 103, wherein the IL18Rα moiety comprises or consists of an amino acid sequence having at least 96% sequence identity to the amino acid sequence of SEQ ID NO: 10.

111. The IL18 receptor agonist of any one of embodiments 1 to 103, wherein the IL18Rα moiety comprises or consists of an amino acid sequence having at least 97% sequence identity to the amino acid sequence of SEQ ID NO:10.

112. The IL18 receptor agonist of any one of embodiments 1 to 103, wherein the IL18Rα moiety comprises or consists of an amino acid sequence having at least 98% sequence identity to the amino acid sequence of SEQ ID NO:10.

113. The IL18 receptor agonist of any one of embodiments 1 to 103, wherein the IL18Rα moiety comprises or consists of an amino acid sequence having at least 99% sequence identity to the amino acid sequence of SEQ ID NO:10.

114. The IL18 receptor agonist of any one of embodiments 1 to 103, wherein the IL18Rα moiety comprises or consists of the amino acid sequence of SEQ ID NO:10.

115. The IL18 receptor agonist of any one of embodiments 1 to 114, wherein the IL18Rα moiety comprises or consists of an amino acid sequence having at least 90% sequence identity to the amino acid sequence of SEQ ID NO:9.

116. The IL18 receptor agonist of any one of embodiments 1 to 114, wherein the IL18Rα moiety comprises or consists of an amino acid sequence having at least 91% sequence identity to the amino acid sequence of SEQ ID NO:9.

117. The IL18 receptor agonist of any one of embodiments 1 to 114, wherein the IL18Rα moiety comprises or consists of an amino acid sequence having at least 92% sequence identity to the amino acid sequence of SEQ ID NO:9.

118. The IL18 receptor agonist of any one of embodiments 1 to 114, wherein the IL18Rα moiety comprises or consists of an amino acid sequence having at least 93% sequence identity to the amino acid sequence of SEQ ID NO:9.

119. The IL18 receptor agonist of any one of embodiments 1 to 114, wherein the IL18Rα moiety comprises or consists of an amino acid sequence having at least 94% sequence identity to the amino acid sequence of SEQ ID NO:9.

120. The IL18 receptor agonist of any one of embodiments 1 to 114, wherein the IL18Rα moiety comprises or consists of an amino acid sequence having at least 95% sequence identity to the amino acid sequence of SEQ ID NO:9.

121. The IL18 receptor agonist of any one of embodiments 1 to 114, wherein the IL18Rα moiety comprises or consists of an amino acid sequence having at least 96% sequence identity to the amino acid sequence of SEQ ID NO:9.

122. The IL18 receptor agonist of any one of embodiments 1 to 114, wherein the IL18Rα moiety comprises or consists of an amino acid sequence having at least 97% sequence identity to the amino acid sequence of SEQ ID NO:9.

123. The IL18 receptor agonist of any one of embodiments 1 to 114, wherein the IL18Rα moiety comprises or consists of an amino acid sequence having at least 98% sequence identity to the amino acid sequence of SEQ ID NO:9.

124. The IL18 receptor agonist of any one of embodiments 1 to 114, wherein the IL18Rα moiety comprises or consists of an amino acid sequence having at least 99% sequence identity to the amino acid sequence of SEQ ID NO:9.

125. The IL18 receptor agonist of any one of embodiments 1 to 114, wherein the IL18Rα moiety comprises or consists of the amino acid sequence of SEQ ID NO:9.

126. The IL18 receptor agonist of any one of embodiments 1 to 125, wherein the IL18 moiety comprises or consists of an amino acid sequence having at least 90% sequence identity to the amino acid sequence of SEQ ID NO:2.

127. The IL18 receptor agonist of any one of embodiments 1 to 125, wherein the IL18 moiety comprises or consists of an amino acid sequence having at least 91% sequence identity to the amino acid sequence of SEQ ID NO:2.

128. The IL18 receptor agonist of any one of embodiments 1 to 125, wherein the IL18 moiety comprises or consists of an amino acid sequence having at least 92% sequence identity to the amino acid sequence of SEQ ID NO:2.

129. The IL18 receptor agonist of any one of embodiments 1 to 125, wherein the IL18 moiety comprises or consists of an amino acid sequence having at least 93% sequence identity to the amino acid sequence of SEQ ID NO:2.

130. The IL18 receptor agonist of any one of embodiments 1 to 125, wherein the IL18 moiety comprises or consists of an amino acid sequence having at least 94% sequence identity to the amino acid sequence of SEQ ID NO:2.

131. The IL18 receptor agonist of any one of embodiments 1 to 125, wherein the IL18 moiety comprises or consists of an amino acid sequence having at least 95% sequence identity to the amino acid sequence of SEQ ID NO:2.

132. The IL18 receptor agonist of any one of embodiments 1 to 125, wherein the IL18 moiety comprises or consists of an amino acid sequence having at least 96% sequence identity to the amino acid sequence of SEQ ID NO:2.

133. The IL18 receptor agonist of any one of embodiments 1 to 125, wherein the IL18 moiety comprises or consists of an amino acid sequence having at least 97% sequence identity to the amino acid sequence of SEQ ID NO:2.

134. The IL18 receptor agonist of any one of embodiments 1 to 125, wherein the IL18 moiety comprises or consists of an amino acid sequence having at least 98% sequence identity to the amino acid sequence of SEQ ID NO:2.

135. The IL18 receptor agonist of any one of embodiments 1 to 125, wherein the IL18 moiety comprises or consists of an amino acid sequence having at least 99% sequence identity to the amino acid sequence of SEQ ID NO:2.

136. The IL18 receptor agonist of any one of embodiments 1 to 125, wherein the IL18 moiety comprises or consists of the amino acid sequence of SEQ ID NO:2.

137. The IL18 receptor agonist of any one of embodiments 1 to 125, wherein the IL18 moiety is a human IL18 mutein.

138. The IL18 receptor agonist of embodiment 137, wherein the human IL18 mutein comprises one or more amino acid substitutions selected from Y1F, Y1H, E6A, E6Q, S7C, S7P, K8E, K8Q, K8Y, S10C, V11I, N14C, N14W, L15C, D17N, Q18L, D23N, D23S, R27Q, P28C, L29V, E31Q, M33C, T34P, D35N, D35E, S36D, S36N, D37N, C38S, C38Q, C38R, C38E, C38L, C38I, C38V, C38K, C38D, R39S, R39T, D40N, N41Q, R44Q, I46V, I49C, S50C, S50Y, M511, M51K, M51Q, M51R, M51L, M51H, M51F, M51Y, K53A, K53D, K53E, K53G, K53H, K531, K53L, K53M, K53N, K53Q, K53R, K53S, K53T, K53V, K53Y, K53F, D54C, S55N, S55Q, S55D, S55E, S55T, Q561, Q56L, P57A, P57E, P57T, P57V, P57Q, P57D, P57Y, P57N, M60I, M60L, M60K, M60Y, M60F, A61C, V62C, T63C, S65C, K67Q, C68S, C68I, C68F, C68Y, C68D, C68N, C68E, C68Q, C68K, E69K, I71M, C76S, C76E, C76K, E77K, I80T, I81L, I81V, N87S, P88C, D90E, K93D, K93N, T95E, K96G, K96Q, S97N, Q103C, Q103E, Q103I, Q103L, H109W, H109Y, D110N, D110Q, D110R, N111D, N111Q, N111S, N111T, N111E, M1131, S119L, A126C, C127S, C127W, C127Y, C127F, C127D, C127E, C127K, D132Q, D132E, L136C, L138C, K139C, E141K, E141Q, L144N, D146F, D146L, D146Y, R147C, R147K, I149V, M150F, M150T, N155C, E156Q, D157A, D157S, and D157N, relative to SEQ ID NO:1.

139. The IL18 receptor agonist of embodiment 137, wherein the human IL18 mutein comprises one or more amino acid substitutions selected from Y1H, Y1R, L5H, L51, L5Y, K8Q, K8R, M51T, M51K, M51D, M51N, M51E, M51R, K53R, K53G, K53S, K53T, S55K, S55R, Q56E, Q56A, Q56R, Q56V, Q56G, Q56K, Q56L, P57L, P57G, P57A, P57K, G59T, G59A, M60K, M60Q, M6OR, M6OL, E77D, Q103E, Q103K, Q103P, Q103A, Q103R, S105R, S105D, S105K, S105N, S105A, D110H, D110K, D110N, D110Q, D110E, D110S, D110G, N111H, N111Y, N111D, N111R, N111S, N111G, M113V, M113R, M113T, M113K, V153I, V153T, V153A, N155K, and N155H relative to SEQ ID NO:1.

140. The IL18 receptor agonist of any one of embodiments 137 to 139, wherein the human IL18 mutein comprises at most one, two, three, four, five, or six amino acid substitutions relative to SEQ ID NO:1.

141. The IL18 receptor agonist of any one of embodiments 1 to 140, which is a heterodimer.

142. The IL18 receptor agonist of any one of embodiments 1 to 141, wherein the first Fc domain comprises a hinge domain.

143. The IL18 receptor agonist of embodiment 142, wherein the hinge domain is an IgG1 hinge domain.

144. The IL18 receptor agonist of embodiment 142, wherein the hinge domain is an IgG4 hinge domain.

145. The IL18 receptor agonist of embodiment 142, wherein the hinge domain is a chimeric hinge domain.

146. The IL18 receptor agonist of embodiment 142, wherein the hinge domain is a modified hinge domain having reduced effector function (e.g., a modified hinge domain as described in Section 6.6.3).

147. The IL18 receptor agonist of any one of embodiments 1 to 146, wherein the second Fc domain comprises a hinge domain.

148. The IL18 receptor agonist of embodiment 147, wherein the hinge domain is an IgG1 hinge domain.

149. The IL18 receptor agonist of embodiment 147, wherein the hinge domain is an IgG4 hinge domain.

150. The IL18 receptor agonist of embodiment 147, wherein the hinge domain is a chimeric hinge domain.

151. The IL18 receptor agonist of embodiment 147, wherein the hinge domain is a modified hinge domain having reduced effector function (e.g., a modified hinge domain as described in Section 6.6.3).

152. The IL18 receptor agonist of any one of embodiments 1 to 151, wherein the first Fc domain comprises a sequence having at least 90%, 91%, 92%, 93%, 94%, 95%, 96%, 97%, 98%, 99%, or 100% sequence identity to an amino acid sequence of any one of SEQ ID NOs:12-30.

153. The IL18 receptor agonist of any one of embodiments 1 to 152, wherein the second Fc domain comprises a sequence having at least 90%, 91%, 92%, 93%, 94%, 95%, 96%, 97%, 98%, 99%, or 100% sequence identity to an amino acid sequence of any one of SEQ ID NOs: 12-30.

154. The IL18 receptor agonist of any one of embodiments 1 to 153, wherein the first Fc domain comprises a knob mutation and the second Fc domain comprises a hole mutation.

155. The IL18 receptor agonist of any one of embodiments 1 to 153, the first Fc domain comprises a hole mutation and the second Fc domain comprises a knob mutation.

156. The IL18 receptor agonist of any one of embodiments 1 to 155, wherein the first Fc domain comprises a star mutation.

157. The IL18 receptor agonist of any one of embodiments 1 to 155, wherein the second Fc domain comprises a star mutation.

158. A nucleic acid or plurality of nucleic acids encoding the IL18 receptor agonist of any one of embodiments 1 to 157.

159. A cell engineered to express the IL18 receptor agonist of any one of embodiments 1 to 157.

160. A cell transfected with one or more expression vectors comprising one or more nucleic acid sequences encoding the IL18 receptor agonist of any one of embodiments 1 to 157 under the control of one or more promoters.

161. A method of producing the IL18 receptor agonist of any one of embodiments 1 to 157, comprising culturing the cell of embodiment 159 or 160 and recovering the IL18 receptor agonist expressed thereby.

162. A method of activating IL18 receptor signaling in a cell or population of cells comprising administering to the cell or population of cells the IL18 receptor agonist of any one of embodiments 1 to 157.

163. The method of embodiment 162, wherein the administration is in vitro.

164. The method of embodiment 162, wherein the administration is in vivo.

165. A pharmaceutical composition comprising the IL18 receptor agonist of any one of embodiments 1 to 157 and an excipient.

166. A method of treating cancer, comprising administering to a subject in need thereof the IL18 receptor agonist of any one of embodiments 1 to 157 or the pharmaceutical composition of embodiment 165.

167. A method of inhibiting growth of a tumor cell in a subject, comprising administering to the subject the IL18 receptor agonist of any one of embodiments 1 to 157 or the pharmaceutical composition of embodiment 165.

168. The method of embodiment 166 or 167, wherein the administration is systemic, optionally intravenous.

169. The method of embodiment 166 or 167, wherein the administration is subcutaneous.

170. The method of any one of embodiments 166 to 169, further comprising administering an anti-PD1 antibody or antigen-binding portion thereof.

171. The method of embodiment 170, wherein the anti-PD1 antibody is an anti-PD1 antibody presented in Table A-2.

172. The method of embodiment 170, wherein the anti-PD1 antibody is MDX-1106 (nivolumab), MK-3475 (pembrolizumab), tislelizumab, retifanlimab, dostarlimab, camrelizumab, balstilimab, cemiplimab, sintilimab, MEDI-0680 (AMP-514), PDR001, or BGB-108.

173. The method of any one of embodiments 166 to 169, further comprising administering an immunotherapeutic.

174. The method of embodiment 173, wherein the immunotherapeutic is a checkpoint inhibitor.

175. The method of any one of embodiments 166 to 173, wherein administration elicits an immune response against a cancerous cell.

176. The method of any one of embodiments 166 to 175, wherein the subject was previously treated for cancer.

177. The method of embodiment 176, wherein the subject was determined to be resistant to the previous treatment.

178. The method of embodiment 176 or 177, wherein the previous treatment was checkpoint inhibitor therapy, optionally an anti-PD1 or anti-PDL1 antibody therapy.

179. A method of localized delivery of an IL18 protein, comprising administering to a subject in need thereof the IL18 receptor agonist of any one of embodiments 1 to 157 or the pharmaceutical composition of embodiment 165.

180. A method of administering to a subject IL18 therapy with reduced systemic exposure and/or reduced systemic toxicity, comprising administering to the subject the IL18 therapy in the form of an IL18 receptor agonist of any one of embodiments 1 to 157 or pharmaceutical composition of embodiment 165.

### 8. EXAMPLES

### 8.1. Materials and Methods

### 8.1.1. Design, Expression, and Purification of Constructs

Constructs encoding the molecules described in Table E1, below, were cloned in ready to use constructs in pcDNA3.4 Topo expression system from Life Technologies (Carlsbad, CA), which were co-transfected into Expi293 cells (ThermoFisher Scientific) following the manufacturer's protocol. 50 ml of cell culture medium was harvested and processed by MabSelect SURE column. Selected constructs were subjected to two-step purification procedures whereby a Mab Select SURE column was used for affinity purification of 50-200 ml of conditioned media as per supplier's instructions. Eluates containing protein of interest were dialyzed into PBSG and subjected to size exclusion chromatography (SEC) using an AKTA PURE System (Cytiva) with S200Increase column.

TABLE E1

| Name | Description | Details and/or sequence(s) |
|---|---|---|
| mIL18 | Wild type murine | NFGRLHCTTAVIRNINDQVLFVDKRQPVFEDMTDIDQSASEPQTRLI |
| | IL18 | IYMYKDSEVRGLAVTLSVKDSKMSTLSCKNKIISFEEMDPPENIDDI |
| | | QSDLIFFQKRVPGHNKMEFESSLYEGHFLACQKEDDAFKLILKKKDE |
| | | NGDKSVMFTLTNLHQS (SEQ ID NO: 4) |
| IL18-Fc | Wild type murine | NFGRLHCTTAVIRNINDQVLFVDKRQPVFEDMTDIDQSASEPQTRLI |
| | IL18 linked N- | IYMYKDSEVRGLAVTLSVKDSKMSTLSCKNKIISFEEMDPPENIDDI |
| | terminal to an Fc | QSDLIFFQKRVPGHNKMEFESSLYEGHFLACQKEDDAFKLILKKKDE |
| | domain | NGDKSVMFTLTNLHQSGGGGSGGGGSGGGGSESKYGPPCPPCPAPPV |
| | | AGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLP |
| | | SSIEKTISKAKGQPREPQVYTLPPSQEEMTKNQVSLTCLVKGFYPSD |
| | | IAVEWESNGQPENNYKTTPPVLDSDGSFFLYSRLTVDKSRWQEGNVF |
| | | SCSVMHEALHNHYTQKSLSLSLGK (SEQ ID NO: 125) |
| Fc-IL18 | Wild type murine | ESKYGPPCPPCPAPPVAGPSVFLFPPKPKDTLMISRTPEVTCVVVDV |
| | IL18 linked C- | SQEDPEVQFNWYVDGVEVHNAKTKPREEQFNSTYRVVSVLTVLHQDW |
| | terminal to an Fc | LNGKEYKCKVSNKGLPSSIEKTISKAKGQPREPQVYTLPPSQEEMTK |
| | domain | NQVSLTCLVKGFYPSDIAVEWESNGQPENNYKTTPPVLDSDGSFFLY |
| | | SRLTVDKSRWQEGNVFSCSVMHEALHNHYTQKSLSLSLGKGGGGSGG |
| | | GGSGGGGSNFGRLHCTTAVIRNINDQVLFVDKRQPVFEDMTDIDQSA |
| | | SEPQTRLIIYMYKDSEVRGLAVTLSVKDSKMSTLSCKNKIISFEEMD |
| | | PPENIDDIQSDLIFFQKRVPGHNKMEFESSLYEGHFLACQKEDDAFK |
| | | LILKKKDENGDKSVMFTLTNLHQS (SEQ ID NO: 126) |
| mIL18(CS2) | IL18 mutein | Polypeptide 1: |
| x Fc | having six amino | HFGRLHCTTAVIRNINDQVLFVDKRQPVFEDMTDIDQSASEPQTRLI |
| | acid substitutions | IYAYGDSRARGKAVTLSVKDSKMSTLSCKNKIISFEEMDPPENIDDI |
| | (N1H, M50A, | QSDLIFFQKRVPGHNKMEFESSLYEGHFLACQKEDDAFKLILKKKDE |
| | K52G, E55R, | NGDKSVMFTLTNLHQSGGGGSGGGGSGGGGSESKYGPPCPPCPAPPV |
| | V56A, and L59K) | AGPSVFLFPPKPKDTLMISRTPEVTCVVVDVSQEDPEVQFNWYVDGV |
| | linked N-terminal | EVHNAKTKPREEQFNSTYRVVSVLTVLHQDWLNGKEYKCKVSNKGLP |
| | to a first Fc | SSIEKTISKAKGQPREPQVYTLPPSQEEMTKNQVSLWCLVKGFYPSD |
| | domain | IAVEWESNGQPENNYKTTPPVLDSDGSFFLYSRLTVDKSRWQEGNVF |
| | associated with a | SCSVMHEALHNHYTQKSLSLSLGK (SEQ ID NO: 127) |
| | second Fc | Polypeptide 2: |
| | domain to form a | ESKYGPPCPPCPAPPVAGPSVFLFPPKPKDTLMISRTPEVTCVVVDV |
| | heterodimer | SQEDPEVQFNWYVDGVEVHNAKTKPREEQFNSTYRVVSVLTVLHQDW |
| | | LNGKEYKCKVSNKGLPSSIEKTISKAKGQPREPQVYTLPPSQEEMTK |
| | | NQVSLSCAVKGFYPSDIAVEWESNGQPENNYKTTPPVLDSDGSFFLV |
| | | SRLTVDKSRWQEGNVFSCSVMHEALHNRFTQKSLSLSPGK (SEQ ID |
| | | NO: 128) |
| (mIL18xRα)- | Configuration 5 | Polypeptide 1: |
| hPD1 | molecule | NFGRLHCTTAVIRNINDQVLFVDKRQPVFEDMTDIDQSASEPQTRLI |
| | (depicted in FIG. | IYMYKDSEVRGLAVTLSVKDSKMSTLSCKNKIISFEEMDPPENIDDI |
| | 2C), where the | QSDLIFFQKRVPGHNKMEFESSLYEGHFLACQKEDDAFKLILKKKDE |
| | IL 18 moiety is | NGDKSVMFTLTNLHQSGGGGSGGGGSESKYGPPCPPCPAPPVAGPSV |
| | wild-type murine | FLFPPKPKDTLMISRTPEVTCVVVDVSQEDPEVQFNWYVDGVEVHNA |
| | IL18, the IL18Rα | KTKPREEQFNSTYRVVSVLTVLHQDWLNGKEYKCKVSNKGLPSSIEK |
| | moiety is D1-D3 | TISKAKGQPREPQVYTLPPSQEEMTKNQVSLWCLVKGFYPSDIAVEW |
| | of IL18Rα, and | ESNGQPENNYKTTPPVLDSDGSFFLYSRLTVDKSRWQEGNVFSCSVM |
| | the targeting | HEALHNHYTQKSLSLSLGK (SEQ ID NO: 129)-linker-VH-CH1 |
| | moiety is an anti- | Polypeptide 2: |
| | PD1 Fab. | KSCIHRSQIHVVEGEPFYLKPCGISAPVHRNETATMRWFKGSASHEY |
| | | RELNNRSSPRVTFHDHTLEFWPVEMEDEGTYISQVGNDRRNWTLNVT |
| | | KRNKHSCFSDKLVTSRDVEVNKSLHITCKNPNYEELIQDTWLYKNCK |
| | | EISKTPRILKDAEFGDEGYYSCVFSVHHNGTRYNITKTVNITVIEGR |
| | | SKVTPAILGPKCEKVGVELGKDVELNCSASLNKDDLFYWSIRKEDSS |
| | | DPNVQEDRKETTTWISEGKLHASKILRFQKITENYLNVLYNCTVANE |
| | | EAIDTKSFVLVRKGGGGSGGGGSESKYGPPCPPCPAPPVAGPSVFLF |
| | | PPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTIS |
| | | KAKGQPREPQVYTLPPSQEEMTKNQVSLSCAVKGFYPSDIAVEWESN |
| | | GQPENNYKTTPPVLDSDGSFFLVSRLTVDKSRWQEGNVFSCSVMHEA |
| | | LHNRFTQKSLSLSPGK (SEQ ID NO: 130)-linker-VH-CH1 |
| | | Polypeptide 3: VL-CL |
| | | Polypeptide 4: VL-CL |
| (mIL18xRα)- | Configuration 5 | Polypeptide 1: |
| Iso | molecule | NFGRLHCTTAVIRNINDQVLFVDKRQPVFEDMTDIDQSASEPQTRLI |
| | (depicted in FIG. | IYMYKDSEVRGLAVTLSVKDSKMSTLSCKNKIISFEEMDPPENIDDI |
| | 2C), where the | QSDLIFFQKRVPGHNKMEFESSLYEGHFLACQKEDDAFKLILKKKDE |
| | IL18 moiety is | NGDKSVMFTLTNLHQSGGGGSGGGGSESKYGPPCPPCPAPPVAGPSV |
| | wild-type murine | FLFPPKPKDTLMISRTPEVTCVVVDVSQEDPEVQFNWYVDGVEVHNA |
| | IL18, the IL18Rα | KTKPREEQFNSTYRVVSVLTVLHQDWLNGKEYKCKVSNKGLPSSIEK |
| | moiety is D1-D3 | TISKAKGQPREPQVYTLPPSQEEMTKNQVSLWCLVKGFYPSDIAVEW |
| | of IL18Rα, and | ESNGQPENNYKTTPPVLDSDGSFFLYSRLTVDKSRWQEGNVFSCSVM |
| | the targeting | HEALHNHYTQKSLSLSLGK (SEQ ID NO: 129)-linker-VH-CH1 |
| | moiety is a Fab | Polypeptide 2: |
| | that does not | KSCIHRSQIHVVEGEPFYLKPCGISAPVHRNETATMRWFKGSASHEY |
| | bind to PD1. | RELNNRSSPRVTFHDHTLEFWPVEMEDEGTYISQVGNDRRNWTLNVT |
| | | KRNKHSCFSDKLVTSRDVEVNKSLHITCKNPNYEELIQDTWLYKNCK |
| | | EISKTPRILKDAEFGDEGYYSCVFSVHHNGTRYNITKTVNITVIEGR |
| | | SKVTPAILGPKCEKVGVELGKDVELNCSASLNKDDLFYWSIRKEDSS |
| | | DPNVQEDRKETTTWISEGKLHASKILRFQKITENYLNVLYNCTVANE |
| | | EAIDTKSFVLVRKGGGGSGGGGSESKYGPPCPPCPAPPVAGPSVFLF |
| | | PPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTIS |
| | | KAKGQPREPQVYTLPPSQEEMTKNQVSLSCAVKGFYPSDIAVEWESN |
| | | GQPENNYKTTPPVLDSDGSFFLVSRLTVDKSRWQEGNVFSCSVMHEA |
| | | LHNRFTQKSLSLSPGK (SEQ ID NO: 130)-linker-VH-CH1 |
| | | Polypeptide 3: VL-CL |
| | | Polypeptide 4: VL-CL |
| (mIL18(DR) | Configuration 5 | Polypeptide 1: |
| x Rα)-hPD1 | molecule | NFGRLHCTTAVIRNINDQVLFVDKRQPVFEDMTDIDQSASEPQTRLI |
| | (depicted in FIG. | IYMYKDSEVRGKAVTLSVKDSKMSTLSCKNKIISFEEMDPPENIDDI |
| | 2C), where the | QSDLIFFQKRVPGHNKMEFESSLYEGHFLACQKEDDAFKLILKKKDE |
| | IL18 moiety is a | NGDKSVMFTLTNLHQSGGGGSGGGGSESKYGPPCPPCPAPPVAGPSV |
| | murine IL18 | FLFPPKPKDTLMISRTPEVTCVVVDVSQEDPEVQFNWYVDGVEVHNA |
| | mutein having an | KTKPREEQFNSTYRVVSVLTVLHQDWLNGKEYKCKVSNKGLPSSIEK |
| | L59K | TISKAKGQPREPQVYTLPPSQEEMTKNQVSLWCLVKGFYPSDIAVEW |
| | substitution, the | ESNGQPENNYKTTPPVLDSDGSFFLYSRLTVDKSRWQEGNVFSCSVM |
| | IL18Rα moiety is | HEALHNHYTQKSLSLSLGK (SEQ ID NO: 131)-linker-VH-CH1 |
| | D1-D3 of IL18Rα, | Polypeptide 2: |
| | and the targeting | KSCIHRSQIHVVEGEPFYLKPCGISAPVHRNETATMRWFKGSASHEY |
| | moiety is an anti- | RELNNRSSPRVTFHDHTLEFWPVEMEDEGTYISQVGNDRRNWTLNVT |
| | PD1 Fab. | KRNKHSCFSDKLVTSRDVEVNKSLHITCKNPNYEELIQDTWLYKNCK |
| | | EISKTPRILKDAEFGDEGYYSCVFSVHHNGTRYNITKTVNITVIEGR |
| | | SKVTPAILGPKCEKVGVELGKDVELNCSASLNKDDLFYWSIRKEDSS |
| | | DPNVQEDRKETTTWISEGKLHASKILRFQKITENYLNVLYNCTVANE |
| | | EAIDTKSFVLVRKGGGGSGGGGSESKYGPPCPPCPAPPVAGPSVFLF |
| | | PPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTIS |
| | | KAKGQPREPQVYTLPPSQEEMTKNQVSLSCAVKGFYPSDIAVEWESN |
| | | GQPENNYKTTPPVLDSDGSFFLVSRLTVDKSRWQEGNVFSCSVMHEA |
| | | LHNRFTQKSLSLSPGK (SEQ ID NO: 130)-linker-VH-CH1 |
| | | Polypeptide 3: VL-CL |
| | | Polypeptide 4: VL-CL |
| (mIL18(DR) | Configuration 5 | Polypeptide 1: |
| x Rα)-Iso | molecule | NFGRLHCTTAVIRNINDQVLFVDKRQPVFEDMTDIDQSASEPQTRLI |
| | (depicted in FIG. | IYMYKDSEVRGKAVTLSVKDSKMSTLSCKNKIISFEEMDPPENIDDI |
| | 2C), where the | QSDLIFFQKRVPGHNKMEFESSLYEGHFLACQKEDDAFKLILKKKDE |
| | IL18 moiety is a | NGDKSVMFTLTNLHQSGGGGSGGGGSESKYGPPCPPCPAPPVAGPSV |
| | murine IL18 | FLFPPKPKDTLMISRTPEVTCVVVDVSQEDPEVQFNWYVDGVEVHNA |
| | mutein having an | KTKPREEQFNSTYRVVSVLTVLHQDWLNGKEYKCKVSNKGLPSSIEK |
| | L59K | TISKAKGQPREPQVYTLPPSQEEMTKNQVSLWCLVKGFYPSDIAVEW |
| | substitution, the | ESNGQPENNYKTTPPVLDSDGSFFLYSRLTVDKSRWQEGNVFSCSVM |
| | IL18Rα moiety is | HEALHNHYTQKSLSLSLGK (SEQ ID NO: 131)-linker-VH-CH1 |
| | D1-D3 of IL18Rα, | Polypeptide 2: |
| | and the targeting | KSCIHRSQIHVVEGEPFYLKPCGISAPVHRNETATMRWFKGSASHEY |
| | moiety is a Fab | RELNNRSSPRVTFHDHTLEFWPVEMEDEGTYISQVGNDRRNWTLNVT |
| | that does not | KRNKHSCFSDKLVTSRDVEVNKSLHITCKNPNYEELIQDTWLYKNCK |
| | bind to PD1. | EISKTPRILKDAEFGDEGYYSCVFSVHHNGTRYNITKTVNITVIEGR |
| | | SKVTPAILGPKCEKVGVELGKDVELNCSASLNKDDLFYWSIRKEDSS |
| | | DPNVQEDRKETTTWISEGKLHASKILRFQKITENYLNVLYNCTVANE |
| | | EAIDTKSFVLVRKGGGGSGGGGSESKYGPPCPPCPAPPVAGPSVFLF |
| | | PPKPKDTLMISRTPEVTCVVVDVSQEDPEVQFNWYVDGVEVHNAKTK |
| | | PREEQFNSTYRVVSVLTVLHQDWLNGKEYKCKVSNKGLPSSIEKTIS |
| | | KAKGQPREPQVYTLPPSQEEMTKNQVSLSCAVKGFYPSDIAVEWESN |
| | | GQPENNYKTTPPVLDSDGSFFLVSRLTVDKSRWQEGNVFSCSVMHEA |
| | | LHNRFTQKSLSLSPGK (SEQ ID NO: 130)-linker-VH-CH1 |
| | | Polypeptide 3: VL-CL |
| | | Polypeptide 4: VL-CL |
| mIL 18-Rα x | Configuration 4 | Polypeptide 1: |
| hPD1 | molecule | NFGRLHCTTAVIRNINDQVLFVDKRQPVFEDMTDIDQSASEPQTRLI |
| | (depicted in FIG. | IYMYKDSEVRGLAVTLSVKDSKMSTLSCKNKIISFEEMDPPENIDDI |
| | 2B), where the | QSDLIFFQKRVPGHNKMEFESSLYEGHFLACQKEDDAFKLILKKKDE |
| | IL18 moiety is | NGDKSVMFTLTNLHQSGGGGSGGGGSGGGGSKSCIHRSQIHVVEGEP |
| | wild-type murine | FYLKPCGISAPVHRNETATMRWFKGSASHEYRELNNRSSPRVTFHDH |
| | IL18, the IL18Rα | TLEFWPVEMEDEGTYISQVGNDRRNWTLNVTKRNKHSCFSDKLVTSR |
| | moiety is D1-D3 | DVEVNKSLHITCKNPNYEELIQDTWLYKNCKEISKTPRILKDAEFGD |
| | of IL18Rα, and | EGYYSCVFSVHHNGTRYNITKTVNITVIEGRSKVTPAILGPKCEKVG |
| | the targeting | VELGKDVELNCSASLNKDDLFYWSIRKEDSSDPNVQEDRKETTTWIS |
| | moiety is an anti- | EGKLHASKILRFQKITENYLNVLYNCTVANEEAIDTKSFVLVRKGGG |
| | PD1 Fab. | GSGGGGSGGGGSESKYGPPCPPCPAPPVAGPSVFLFPPKPKDTLMIS |
| | | RTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQV |
| | | YTLPPSQEEMTKNQVSLWCLVKGFYPSDIAVEWESNGQPENNYKTTP |
| | | PVLDSDGSFFLYSRLTVDKSRWQEGNVFSCSVMHEALHNHYTQKSLS |
| | | LSLGK (SEQ ID NO: 132) |
| | | Polypeptide 2:VH- |
| | | CH1-CPPCPAPPVAGPSVFLFPPKPKDTLMISRTPEVTCVVVDVSQE |
| | | DPEVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQV |
| | | SLSCAVKGFYPSDIAVEWESNGQPENNYKTTPPVLDSDGSFFLVSRL |
| | | TVDKSRWQEGNVFSCSVMHEALHNRFTQKSLSLSPGK (SEQ ID NO: |
| | | 133) |
| | | Polypeptide 3: VL-CL |

### 8.1.2. Engineering of Reporter EL4 Cells

The mouse T-cell lymphoma cell line EL4 was transduced with Nuclear Factor kappa-light-chain-enhancer of activated B cells (NFkB) response element driven luciferase reporter construct. A single cell clone, EL4/NFkB-Luc Clone 12 (also referred to as simply EL4/NFkB-Luc), exhibiting high responsiveness to mouse IL18, was identified, and maintained in Dulbecco's Modified Eagle's Medium supplemented with 10% horse serum, 2 mM L-Glutamine-Penicillin-Streptomycin, 1 mM Sodium Pyruvate and 7 μg/mL Puromycin.

CRISPR-Cas9 technology was used to eliminate mouse PD1 from EL4/NFkB-Luc cells. The resulting cell line, EL4/NFkB-Luc/mPD1^{KO}, was established after sorting for mouse PD1-negative cells.

EL4/NFkB-Luc/mPD1^{KO} cells were engineered to stably express human PD1 (amino acids M1-L288 of accession number NP_005009.2, with a 2Q→E mutation). After selection in media supplemented with 1 mg/mL G418, EL4/NFkB-Luc/mPD1^{KO}/hPD1^{OE} cells were maintained in Dulbecco's Modified Eagle's Medium supplemented with 10% horse serum, 2 mM L-Glutamine-Penicillin-Streptomycin, 1 mM Sodium Pyruvate and 7 μg/mL Puromycin and 1 mg/mL G418.

### 8.1.3. FACS Binding Assay

To analyze binding to cell surface targets, HEK293T cells stably expressing hPD1 were collected and resuspended in FACS buffer (PBS+2% FBS+0.5 mM EDTA+0.02% Sodium Azide). For each binding assay, 100,000-200,000 cells were incubated with serial dilutions of IL18 receptor agonists in FACS buffer at 4° C. for 30 minutes. Cells were then washed twice with FACS buffer, and incubated with 1:2000 dilution of A647-F(ab)′2 anti-human IgG Fc fragment (Jackson ImmunoResearch Laboratories) for 30 minutes at 4° C. At the end of the incubation, cells were washed twice with FACS buffer and analyzed on BD FACSCelesta™ flow cytometer.

### 8.1.4. Luciferase Activity Assay

RPMI1640 media supplemented with 2 mM L-Glutamine/Penicillin/Streptomycin+10% FBS was used as the assay medium to prepare cell suspensions and dilutions of IL18 receptor agonists or control constructs.

The day of the assay, EL4/NFkB-Luc, EL4/NFkB-Luc/mPD1^{KO} and EL4/NFkB-Luc/mPD1^{KO}/hPd1^{OE} reporter cells were added at a density of 2.5×10⁴ cells/well to 96-well white flat bottom plates. For one assay, mIL18, IL18 receptor agonists, and control constructs were prepared in assay media and titrated from 100 nM to 256fM in a 5-fold dilution following a 9-point dilution range, with the 10th point, plotted at 51fM, containing no recombinant protein. For another assay, mIL18 constructs were prepared in assay media following an 11-point 4-fold dilution from 50 nM to 48.3fM, with the 12th point, plotted at 12fM, containing no mIL18 construct. The mIL18 chimeric constructs were tested in presence or absence of 10 nM mIL18BP. Titrated constructs were added to wells containing reporter cells in the presence or absence of 10 nM mIL18BP. Plates were incubated for 5 hours at 37° C. and 5% CO₂ before the addition of 100 μL ONE-Glo™ (Promega) reagent to lyse cells and detect luciferase activity. The emitted light was captured in relative light units (RLU) on a multilabel plate reader Envision (PerkinElmer). All serial dilutions were tested in duplicates.

### 8.1.5. In Vivo Administration of Constructs

Female hPD1/hLAG3 mice (20 weeks old) (Burova et al., 2019) were implanted subcutaneously (s.c.) into the right flank with 3×10⁵ MC-38 tumor cells. Mice were randomized into treatment groups (n=7-8 mice per group) when average tumor sizes reached 65 mm³ in volume. On the day of randomization (day 0) and three days post-randomization (day 3), mice were injected intraperitoneally (i.p.) with 2.5 mpk of the assigned construct. Blood samples were collected on days 1, 3, and 6 for further analyses. Body weight and tumor volume changes were assessed twice a week for four weeks post-randomization.

### 8.1.6. In Vivo Tumor Growth Inhibition Assay

Tumor volume was expressed in mm³ using the formula: V=0.5×a×b² where a and b were the long and short diameters of the tumor, respectively. Tumor sizes were monitored twice weekly. All procedures were conducted according to the guidelines of the Regeneron Institutional Animal Care and Use Committee. All data were analyzed using GraphPad Prism and tumor sizes were graphed as mean±SEM.

8.1.7. Immunoprofiling of Blood from Treated Mice

All blood samples were collected through retro-orbital bleeding. For plasma cytokine measurement, day 1 and 3 blood samples were centrifuged at 9000 RCF for 10 minutes and the supernatant was collected as plasma. Levels of proinflammatory cytokines in plasma were measured using the V-Plex Proinflammatory Panel 1 Mouse Kit (Meso Scale Discovery) according to the manufacturer's instructions. For flow cytometric analysis, Day 6 blood samples were subjected to red blood cell lysis using ACK buffer. After RBC lysis, WBCs were counted and stained with antibody cocktails diluted in BD Horizon Brilliant Buffer. Antibody clones used for staining were: CD45 (30-F11), CD90.2 (53-2.1), CD8a (53-6.7), CD4 (GK1.5 or RM4-4), CD19 (1D3), NK1.1 (PK136), Ki-67 (16A8), human PD-1 (EH12.2H7), CD44 (IM7), CD62L (MEL-14), Foxp3 (MF23), CD25 (PC61), TCF1 (C63D9), CD11b (M1/70), Ly6G (1A8), Ly6C (HK1.4), F4/80 (T45-2342). All stained samples were analyzed on a Cytek Aurora flow cytometer. The raw data were processed using FlowJo v10.

### 8.2. Example 1: Optimization of IL18 Moiety Orientation

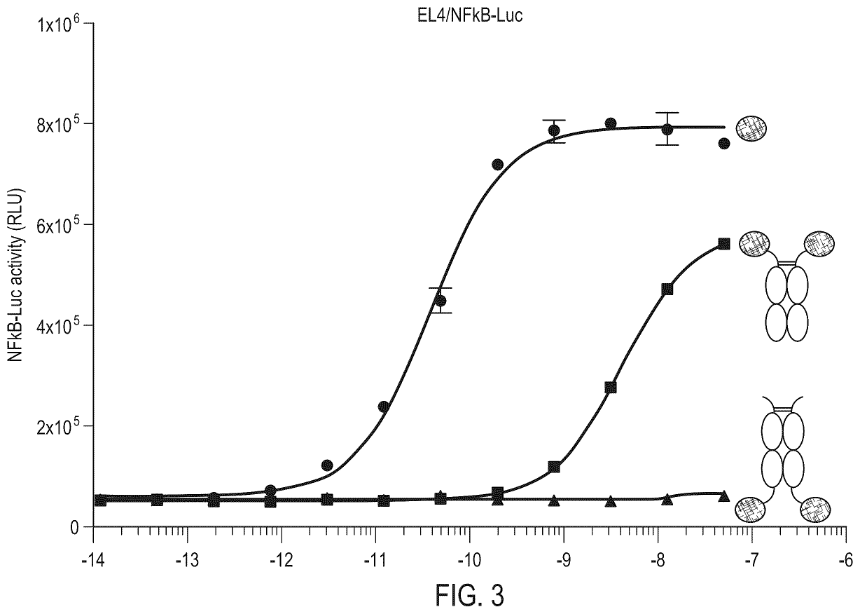

IL18 receptor agonists comprising an IL18 moiety either at the N- or C-terminus of an Fc domain, IL18-Fc or Fc-IL18 (depicted in FIG. 3), were expressed and purified as described in Section 8.1.1. The activity of the IL18 receptor agonists was evaluated via luciferase assay as described in Section 8.1.4.

The IL18 receptor agonist comprising a C-terminal IL18 moiety displayed no detectable luciferase activity in EL4/NFkB-Luc cells at any of the concentrations evaluated. The IL18 receptor agonist comprising an N-terminal IL18 moiety displayed a dose-dependent activity in EL4/NFkB-Luc cells (FIG. 3). These results suggested that maintaining a free N-terminus was important for the bioactivity of IL18 constructs.

### 8.3. Example 2: Expression, Purification, and Binding Activity of IL18 Receptor Agonists

Based on the results obtained in Section 8.2, IL18 receptor agonists were designed to comprise an IL18 moiety at the N-termini. The constructs were designed to further comprise cis- or trans-oriented masking moieties and Fab arms as targeting moieties. All cis- or trans-masked IL18 receptor agonist constructs with Fab arms were produced and purified as described in Section 8.1.1. The binding activity of PD-1-targeted, IL18 receptor agonists to cell surface PD-1 was assessed with FACS as described in Section 8.1.3.

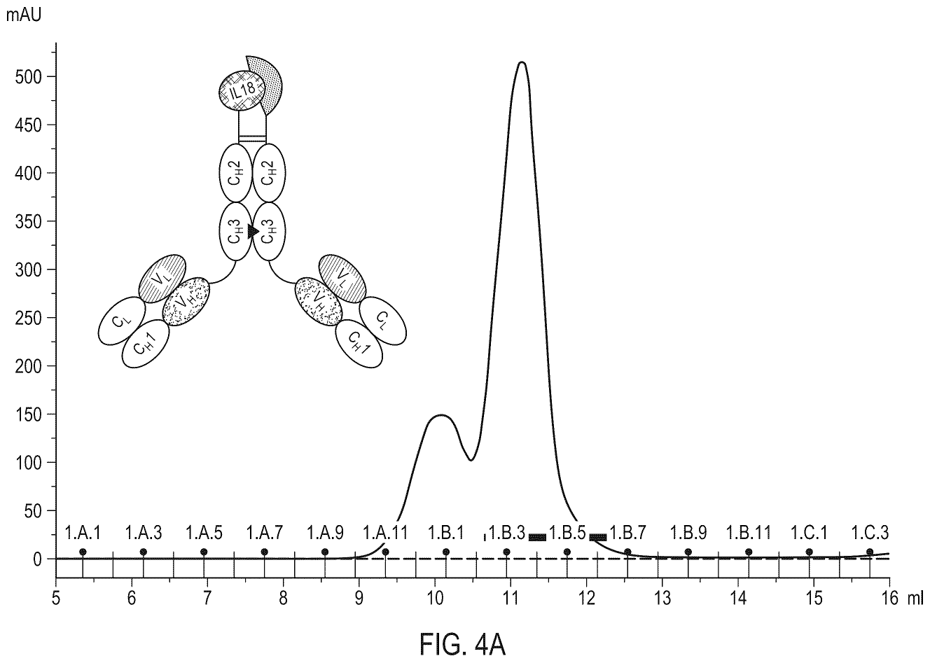

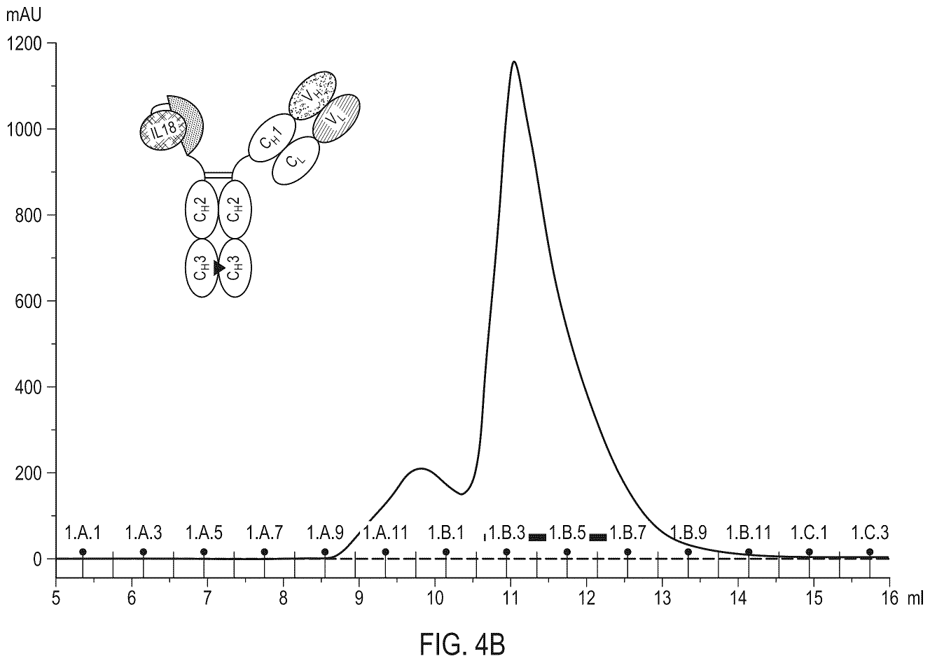

Standard SEC purification resulted in the removal of the majority of high molecular weight (HMW) species from samples of trans-masked (FIG. 4A) and cis-masked (FIG. 4B) IL18 receptor agonist constructs with Fab arms.

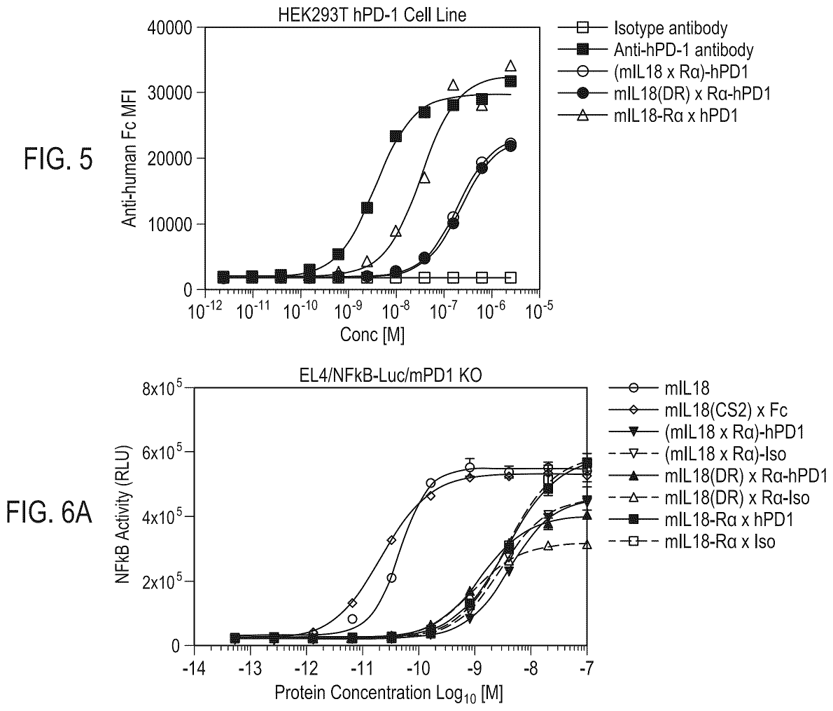

Both cis- and trans-masked IL18 receptor agonist constructs with PD1-targeting Fab arms displayed dose-dependent binding to HEK29T cells overexpressing huPD-1. There was no difference in binding activity between trans-masked IL18 receptor agonists that comprised a wildtype IL18 moiety and trans-masked IL18 receptor agonists that comprised a decoy resistant (DR) IL18 moiety (FIG. 5).

### 8.4. Example 3: Target-Dependent Activity of IL18 Receptor Agonists

The activity of the IL18 receptor agonists was evaluated in EL4/NFkB-Luc/mPD1^{KO} and EL4/NFkB-Luc/mPD1^{KO}/hPd1^{OE} reporter cells in the presence or absence of mIL18BP as described in Section 8.1.4 and compared to the activity of free mIL18.

Compared to recombinant mIL18 or a decoy resistant IL18 mutein-Fc fusion construct (IL18(CS2)×Fc), all non-targeted or PD1-targeted, masked IL18 constructs displayed attenuated activities on EL4/NFkB-Luc/mPD1^{KO} cells in the absence of mIL18BP (FIG. 6A). The presence of mIL18BP did not affect the activity of the IL18(CS2)×Fc or IL18(DR)-containing constructs (mIL18(DR)×Rα-hPD1 and mIL18(DR)×Rα-Iso) but attenuated the activity of mIL18 and all non-targeted and PD1-targeted IL18 receptor agonists in EL4/NFkB-Luc/mPD1^{KO} cells (FIG. 6B).

In EL4/NFkB-Luc/mPD1^{KO}/hPd1^{OE} cells, the activities of PD1-targeted IL18 receptor agonists were relatively higher than those of isotype control IL18 receptor agonists in the absence of mIL18BP (FIG. 6C). In the presence of IL18BP, PD1 targeting was associated with partial recovery of activity relative to isotype control IL18 agonist constructs (FIG. 6D).

### 8.5. Example 4: In Vivo Administration of IL18 Receptor Agonists in Mice

Mice were inoculated with tumor cells, randomized into treatment groups and i.p. injected with assigned IL18 receptor agonists or control constructs as described in Section 8.1.5. The changes in body weight were monitored throughout the assessment and blood samples were collected for evaluation of cytokine release associated with construct administration, and immune cell profiling as described in Section 8.1.7.

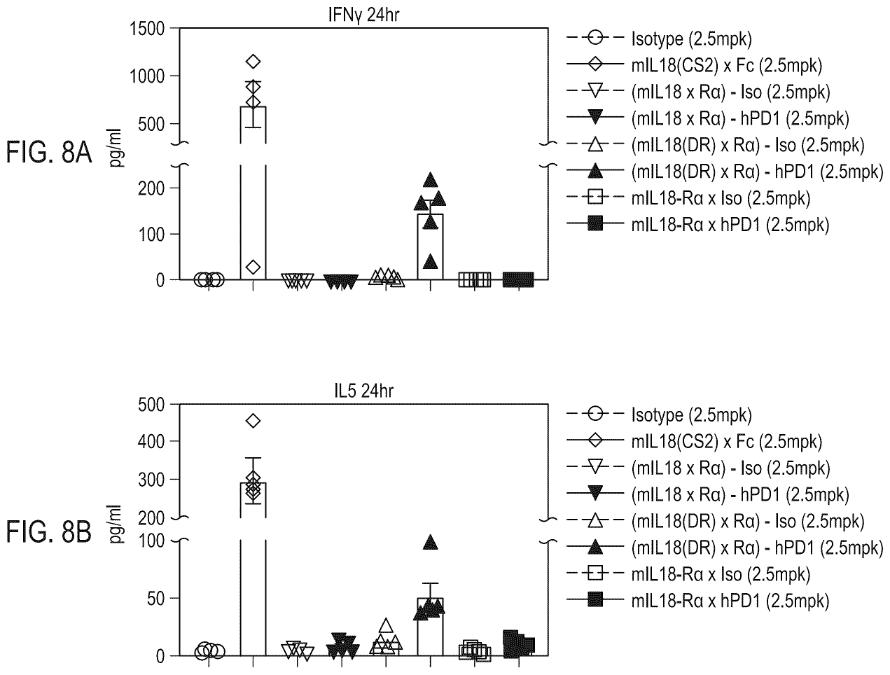

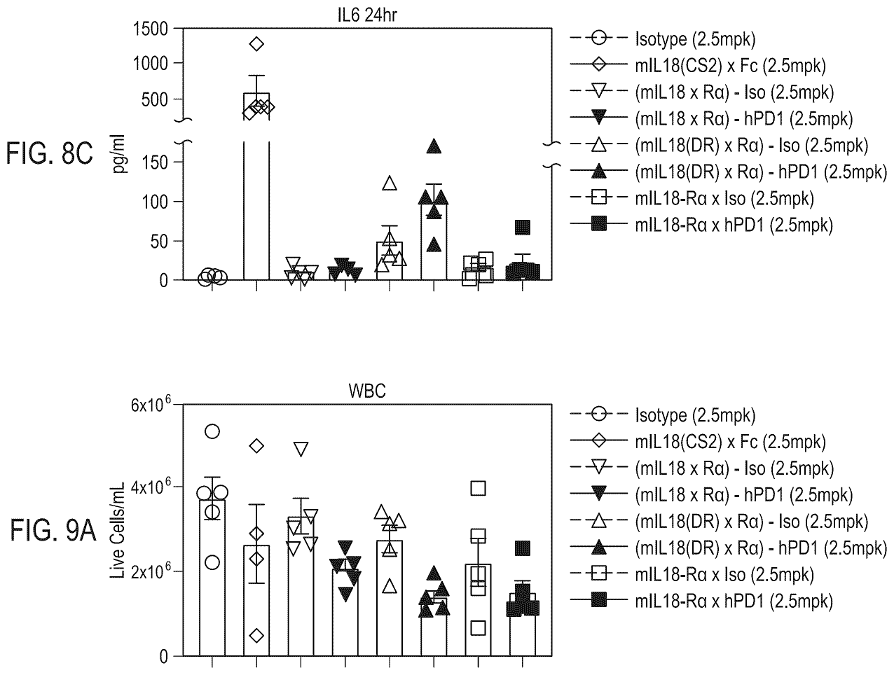

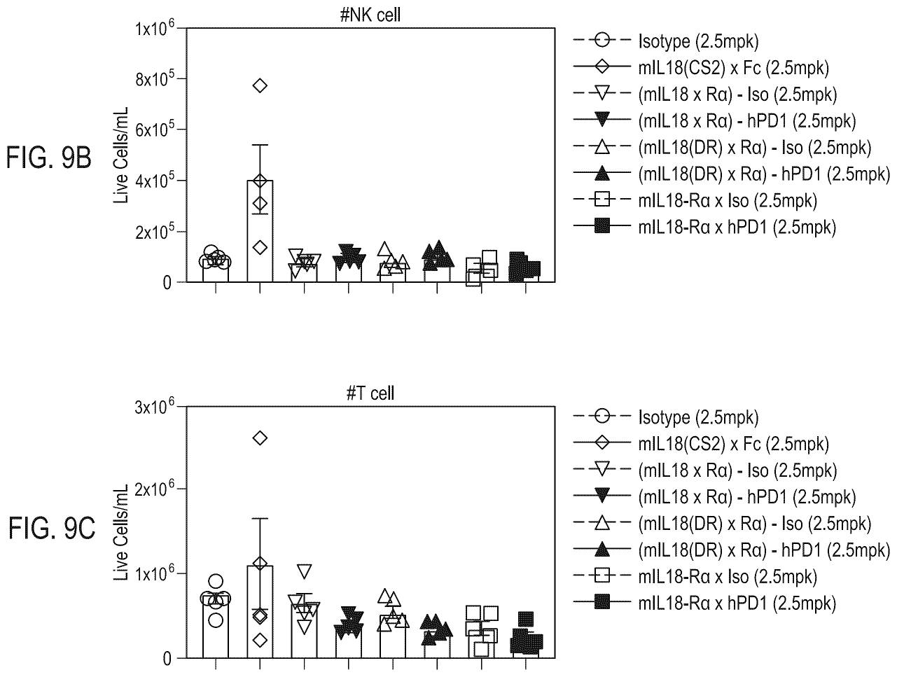

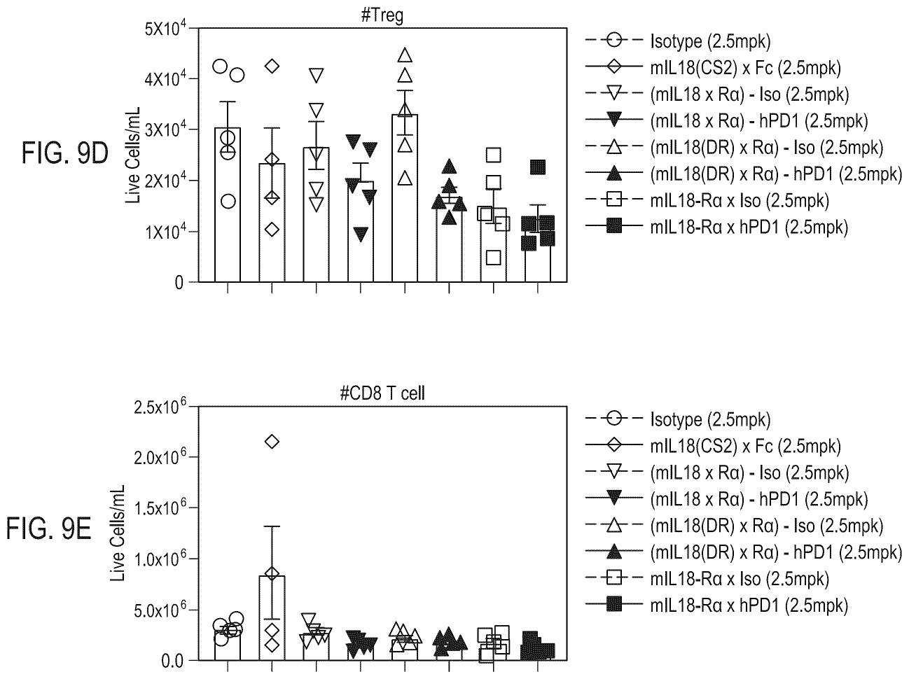

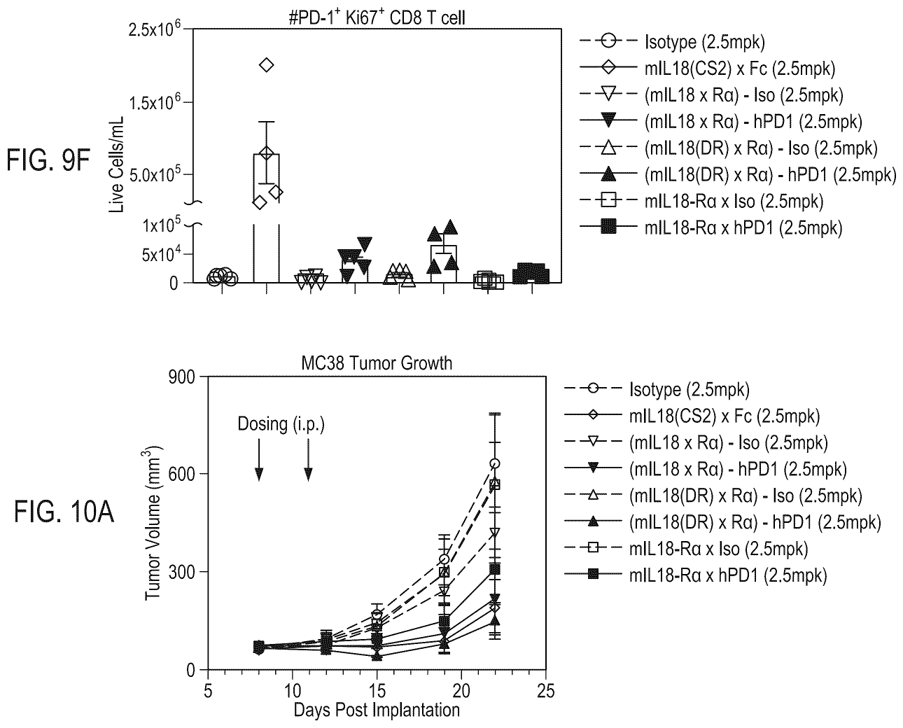

While mIL18(CS2)×Fc treatment was associated with loss of body weight (FIGS. 7A and 7B), none of the masked IL18 receptor agonists led to loss of body weight (FIGS. 7A, 7C-7H). Assessment of plasma cytokines revealed high cytokine levels in mice treated with mIL18(CS2)×Fc. In contrast, minimal cytokine release was detected in mice treated with trans or cis-masked mIL18, regardless of whether they're hPD1-targeted or not. A modest increase of cytokines was observed in mice treated with (mIL18(DR)×Rα)-hPD1. (FIGS. 8A-8C). Similarly, mIL18(CS2)×Fc was associated with increases in NK cells, CD8⁺ T cells, and PD1+Ki67+CD8+ T cells, whereas the masked IL18 receptor agonists had little to no effect on NK and CD8+ T cells, with a moderate increase of PD1+Ki67+CD8+ T cells in the presence PD1 targeting (FIGS. 9A-9F). Taken together, these results suggest that masked IL18 receptor agonists have improved in vivo safety profiles relative to mIL18(CS2)×Fc.

### 8.6. Example 5: Anti-Tumor Efficacy of IL18 Receptor Agonists

Mice were inoculated with tumor cells, randomized into treatment groups and i.p. injected with assigned IL18 receptor agonists or control constructs as described in Section 8.1.5. Tumor volumes were assessed twice per week for the duration of assessments as described in Section 8.1.6.

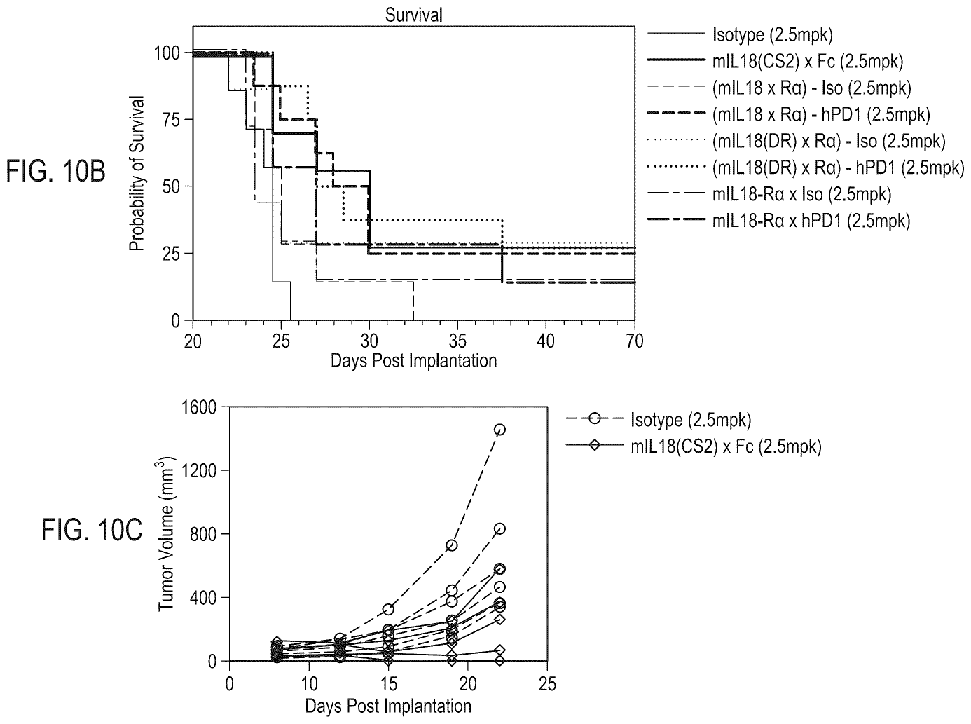

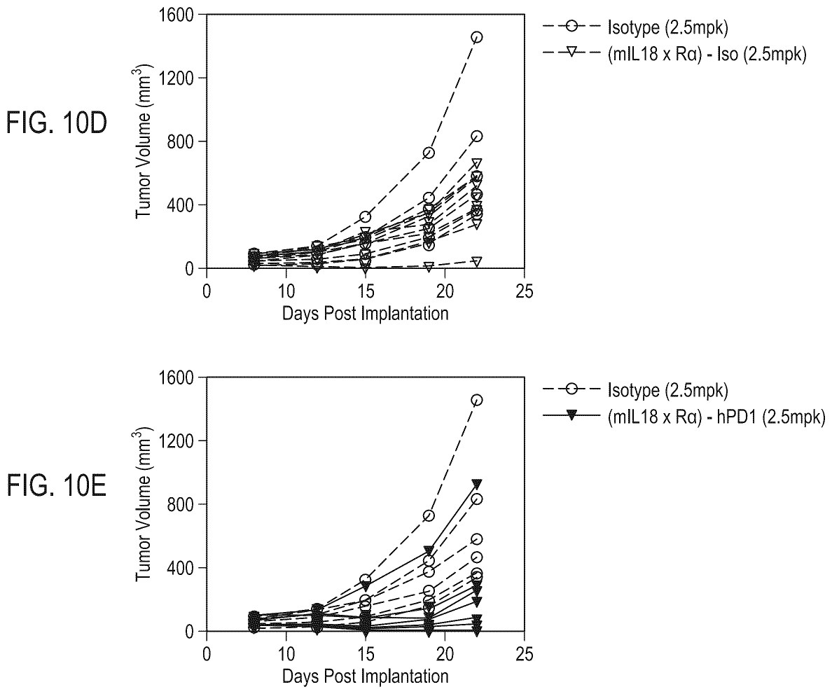

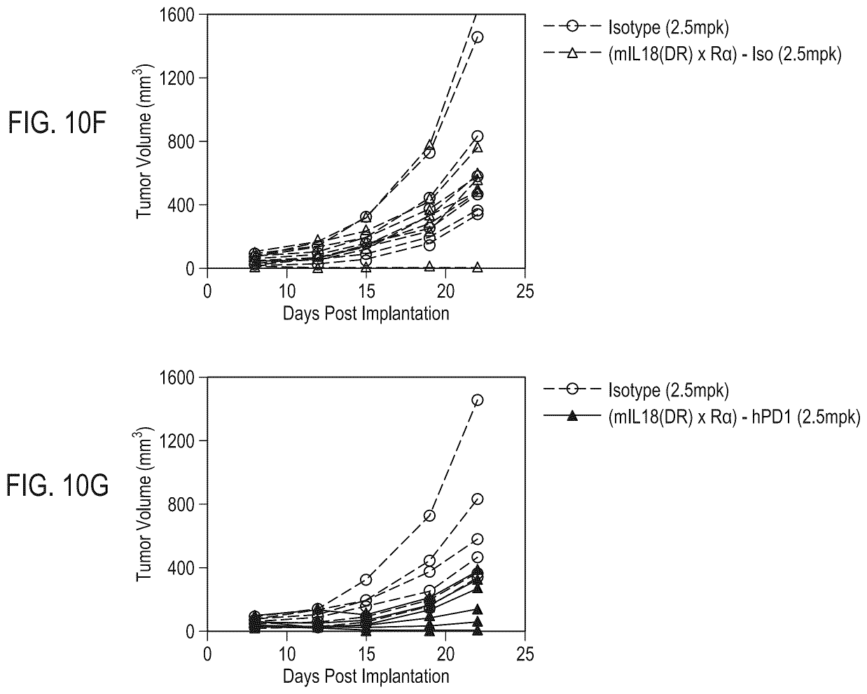

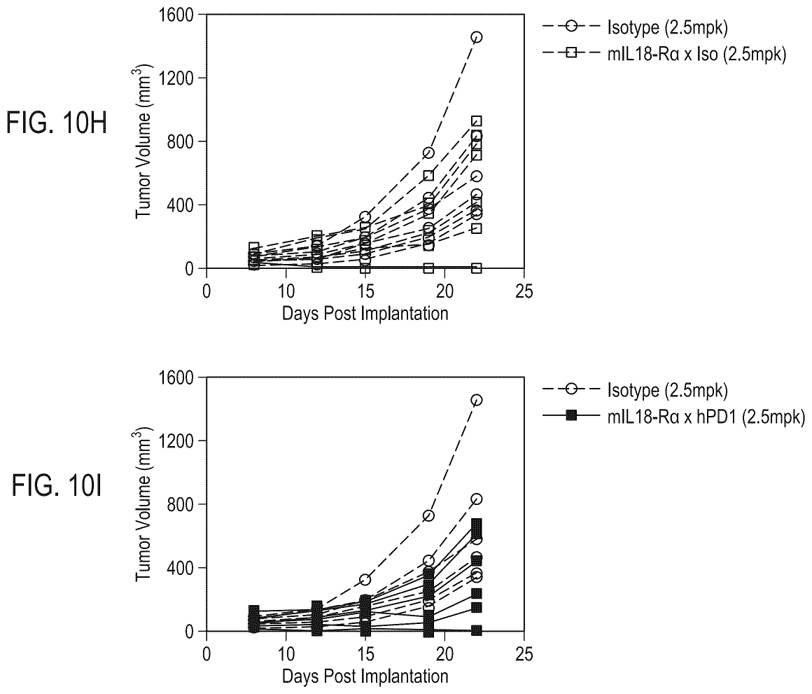

Mice treated with PD1-targeted IL18 receptor agonists had slower rates of tumor growth (FIG. 10A) and higher probabilities of survival (FIG. 10B) than mice treated with non-targeted IL18 receptor agonists. At the dose evaluated, the highest levels of anti-tumor efficacy and survival probabilities were associated with trans-masked PD1-targeted IL18 receptor agonists and mIL18(CS2)×Fc (FIGS. 10A-10I). Taken together with the results in Section 8.5, these results suggest that tumor targeted and masked IL18 receptor agonists have improved safety profiles over mIL18(CS2) while providing similar levels of anti-tumor efficacy.

### 7. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An IL18 receptor agonist comprising, on a first polypeptide chain and a second polypeptide chain dimerized through a first Fc domain and a second Fc domain:
(a) an IL18 moiety N-terminal to the first Fc domain or the second Fc domain; and
(b) an IL18Rα moiety N-terminal to the first Fc domain or the second Fc domain.

2.-14. (canceled)

15. The IL18 receptor agonist of claim 1, wherein:
(a) the first polypeptide chain comprises, in N- to C-terminal orientation, the IL18 moiety and the first Fc domain; and
(b) the second polypeptide chain comprises, in N- to C-terminal orientation, the IL18Rα moiety and the second Fc domain.

16. The IL18 receptor agonist of claim 15, wherein the first polypeptide chain lacks an IL18Rα moiety.

17. The IL18 receptor agonist of claim 15, wherein the second polypeptide chain lacks an IL18 moiety.

18. The IL18 receptor agonist of claim 15, further comprising a targeting moiety.

19. The IL18 receptor agonist of claim 18, wherein the targeting moiety or a component thereof is C-terminal to the first Fc domain.

20.-21. (canceled)

22. The IL18 receptor agonist of claim 1, wherein:
(a) the first polypeptide chain comprises, in N- to C-terminal orientation, the IL18 moiety, the IL18Rα moiety, and the first Fc domain; and
(b) the second polypeptide chain comprises the second Fc domain.

23. The IL18 receptor agonist of claim 22, which is monovalent for the IL18 moiety.

24. The IL18 receptor agonist of claim 22, which is bivalent for the IL18 moiety.

25. The IL18 receptor agonist of claim 23, which is monovalent for the IL18Rα moiety.

26. The IL18 receptor agonist of claim 24, which is bivalent for the IL18Rα moiety.

27. The IL18 receptor agonist of claim 22, wherein the second polypeptide chain further comprises, N-terminal to the second Fc domain, an additional IL18 moiety and an additional IL18Rα moiety.

28. The IL18 receptor agonist of claim 27, wherein the additional IL18 moiety is N-terminal to the additional IL18Rα moiety.

29. The IL18 receptor agonist of claim 22, further comprising a targeting moiety.

30. The IL18 receptor agonist of claim 29, wherein the targeting moiety or a component thereof is N-terminal to the second Fc domain.

31.-40. (canceled)

41. The IL18 receptor agonist of claim 1, wherein the IL18Rα moiety comprises or consists of an amino acid sequence having at least 90% sequence identity to the amino acid sequence of SEQ ID NO:8.

42. The IL18 receptor agonist of claim 1, wherein the IL18Rα moiety comprises or consists of an amino acid sequence having at least 90% sequence identity to the amino acid sequence of SEQ ID NO: 10.

43. The IL18 receptor agonist of claim 1, wherein the IL18Rα moiety comprises or consists of an amino acid sequence having at least 90% sequence identity to the amino acid sequence of SEQ ID NO:9.

44. The IL18 receptor agonist of claim 1, wherein the IL18 moiety comprises or consists of an amino acid sequence having at least 90% sequence identity to the amino acid sequence of SEQ ID NO:2.

45. The IL18 receptor agonist of claim 1, wherein the IL18 moiety is a human IL18 mutein having at most six amino acid substitutions relative to SEQ ID NO: 1.

46. The IL18 receptor agonist of claim 1, wherein (a) the first Fc domain comprises a sequence having at least 90% sequence identity to an amino acid sequence of any one of SEQ ID NOs: 12-30 and/or (b) the second Fc domain comprises a sequence having at least 90% sequence identity to an amino acid sequence of any one of SEQ ID NOs: 12-30.

47. A nucleic acid or plurality of nucleic acids encoding the IL18 receptor agonist of claim 1.

48. A cell transfected with one or more expression vectors comprising one or more nucleic acid sequences encoding the IL18 receptor agonist of claim 1 under the control of one or more promoters.

49. A method of producing an IL18 receptor agonist comprising culturing the cell of claim 48 and recovering the IL18 receptor agonist expressed thereby.

50. A method of activating IL18 receptor signaling in a cell or population of cells comprising administering to the cell or population of cells the IL18 receptor agonist of claim 1.

51. A pharmaceutical composition comprising the IL18 receptor agonist of claim 1 and an excipient.

52. A method of treating cancer or inhibiting growth of a tumor cell in a subject, comprising administering to a subject in need thereof the IL18 receptor agonist of claim 1.

53.-55. (canceled)


SEQUENCE LISTING (136 sequences)

SEQ ID NO: 1 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 3 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 4 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 5 - protein, 311 aa, Homo sapiens
AESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTR

SEQ ID NO: 6 - protein, 96 aa, Homo sapiens
AESCTSRPHITVVEGEPFYLKHCSCSLAHEIETTTKSWYKSSGSQEHVELNPRSSSRIALHDCVLEFWPVELNDTGSYFFQMKNYTQKWKLNVIRR

SEQ ID NO: 7 - protein, 95 aa, Homo sapiens
NKHSCFTERQVTSKIVEVKKFFQITCENSYYQTLVNSTSLYKNCKKLLLENNKNPTIKKNAEFEDQGYYSCVHFLHHNGKLFNITKTFNITIVED

SEQ ID NO: 8 - protein, 107 aa, Homo sapiens
RSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRK

SEQ ID NO: 9 - protein, 298 aa, Homo sapiens
AESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRK

SEQ ID NO: 10 - protein, 184 aa, Homo sapiens
KKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRK

SEQ ID NO: 11 - protein, 307 aa, Mus musculus
KSCIHRSQIHVVEGEPFYLKPCGISAPVHRNETATMRWFKGSASHEYRELNNRSSPRVTFHDHTLEFWPVEMEDEGTYISQVGNDRRNWTLNVTKRNKHSCFSDKLVTSRDVEVNKSLHITCKNPNYEELIQDTWLYKNCKEISKTPRILKDAEFGDEGYYSCVFSVHHNGTRYNITKTVNITVIEGRSKVTPAILGPKCEKVGVELGKDVELNCSASLNKDDLFYWSIRKEDSSDPNVQEDRKETTTWISEGKLHASKILRFQKITENYLNVLYNCTVANEEAIDTKSFVLVRKEIPDIPGHVFTG

SEQ ID NO: 12 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 19 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 24 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 26 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 28 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 30 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 15 aa, synthetic construct
GGGGSGGGGSGGGGS

SEQ ID NO: 32 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 36 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 37 - protein, 4 aa, synthetic construct
CPPC

SEQ ID NO: 38 - protein, 4 aa, Homo sapiens
CPSC

SEQ ID NO: 39 - protein, 20 aa, synthetic construct
EPKSCDKTHTCPPCPAPPVA

SEQ ID NO: 40 - protein, 17 aa, synthetic construct
ESKYGPPCPPCPAPPVA

SEQ ID NO: 41 - protein, 15 aa, synthetic construct
CPPCPAPGGGGPSVF

SEQ ID NO: 42 - protein, 14 aa, synthetic construct
CPPCPAPGGGPSVF

SEQ ID NO: 43 - protein, 13 aa, synthetic construct
CPPCPAPGGPSVF

SEQ ID NO: 44 - protein, 12 aa, synthetic construct
CPPCPAPGPSVF

SEQ ID NO: 45 - protein, 11 aa, synthetic construct
  VARIANT 1..10: note = This region may encompass 1-10 residues
GGGGGGGGGGS

SEQ ID NO: 46 - protein, 11 aa, synthetic construct
  VARIANT 2..11: note = This region may encompass 1-10 residues
SGGGGGGGGGG

SEQ ID NO: 47 - protein, 5 aa, synthetic construct
GGGGS

SEQ ID NO: 48 - protein, 4 aa, synthetic construct
GGGG

SEQ ID NO: 49 - protein, 5 aa, synthetic construct
GGGGG

SEQ ID NO: 50 - protein, 6 aa, synthetic construct
GGGGGG

SEQ ID NO: 51 - protein, 7 aa, synthetic construct
GGGGGGG

SEQ ID NO: 52 - protein, 8 aa, synthetic construct
GGGGGGGG

SEQ ID NO: 53 - protein, 9 aa, synthetic construct
GGGGGGGGG

SEQ ID NO: 54 - protein, 4 aa, synthetic construct
GGGS

SEQ ID NO: 55 - protein, 5 aa, synthetic construct
GSGGS

SEQ ID NO: 56 - protein, 5 aa, synthetic construct
AAAGG

SEQ ID NO: 57 - protein, 5 aa, synthetic construct
ADAAP

SEQ ID NO: 58 - protein, 11 aa, synthetic construct
ADAAPTVSIFP

SEQ ID NO: 59 - protein, 12 aa, synthetic construct
ADAAPTVSIFPP

SEQ ID NO: 60 - protein, 6 aa, synthetic construct
AKTTAP

SEQ ID NO: 61 - protein, 13 aa, synthetic construct
AKTTAPSVYPLAP

SEQ ID NO: 62 - protein, 17 aa, synthetic construct
AKTTPKLEEGEFSEARV

SEQ ID NO: 63 - protein, 9 aa, synthetic construct
AKTTPKLGG

SEQ ID NO: 64 - protein, 6 aa, synthetic construct
AKTTPP

SEQ ID NO: 65 - protein, 13 aa, synthetic construct
AKTTPPSVTPLAP

SEQ ID NO: 66 - protein, 6 aa, synthetic construct
ASTKGP

SEQ ID NO: 67 - protein, 26 aa, synthetic construct
ASTKGPSVFPLAPASTKGPSVFPLAP

SEQ ID NO: 68 - protein, 14 aa, synthetic construct
EGKSSGSGSESKST

SEQ ID NO: 69 - protein, 15 aa, synthetic construct
GEGESGEGESGEGES

SEQ ID NO: 70 - protein, 20 aa, synthetic construct
GEGESGEGESGEGESGEGES

SEQ ID NO: 71 - protein, 15 aa, synthetic construct
GEGGSGEGGSGEGGS

SEQ ID NO: 72 - protein, 15 aa, synthetic construct
GENKVEYAPALMALS

SEQ ID NO: 73 - protein, 15 aa, synthetic construct
GGEGSGGEGSGGEGS

SEQ ID NO: 74 - protein, 15 aa, synthetic construct
GGGESGGEGSGEGGS

SEQ ID NO: 75 - protein, 15 aa, synthetic construct
GGGESGGGESGGGES

SEQ ID NO: 76 - protein, 10 aa, synthetic construct
GGGGSGGGGS

SEQ ID NO: 77 - protein, 20 aa, synthetic construct
GGGGSGGGGSGGGGSGGGGS

SEQ ID NO: 78 - protein, 15 aa, synthetic construct
GGGKSGGGKSGGGKS

SEQ ID NO: 79 - protein, 15 aa, synthetic construct
GGGKSGGKGSGKGGS

SEQ ID NO: 80 - protein, 5 aa, synthetic construct
GGGSG

SEQ ID NO: 81 - protein, 15 aa, synthetic construct
GGKGSGGKGSGGKGS

SEQ ID NO: 82 - protein, 4 aa, synthetic construct
GGSG

SEQ ID NO: 83 - protein, 5 aa, synthetic construct
GGSGG

SEQ ID NO: 84 - protein, 9 aa, synthetic construct
GGSGGGGSG

SEQ ID NO: 85 - protein, 13 aa, synthetic construct
GGSGGGGSGGGGS

SEQ ID NO: 86 - protein, 15 aa, synthetic construct
GHEAAAVMQVQYPAS

SEQ ID NO: 87 - protein, 15 aa, synthetic construct
GKGGSGKGGSGKGGS

SEQ ID NO: 88 - protein, 15 aa, synthetic construct
GKGKSGKGKSGKGKS

SEQ ID NO: 89 - protein, 20 aa, synthetic construct
GKGKSGKGKSGKGKSGKGKS

SEQ ID NO: 90 - protein, 15 aa, synthetic construct
GKPGSGKPGSGKPGS

SEQ ID NO: 91 - protein, 21 aa, synthetic construct
GKPGSGKPGSGKPGSGKPSGS

SEQ ID NO: 92 - protein, 15 aa, synthetic construct
GPAKELTPLKEAKVS

SEQ ID NO: 93 - protein, 12 aa, synthetic construct
GSAGSAAGSGEF

SEQ ID NO: 94 - protein, 5 aa, synthetic construct
GSGGG

SEQ ID NO: 95 - protein, 5 aa, synthetic construct
GSGSG

SEQ ID NO: 96 - protein, 4 aa, synthetic construct
GSSG

SEQ ID NO: 97 - protein, 10 aa, synthetic construct
GSSGGSGGSG

SEQ ID NO: 98 - protein, 11 aa, synthetic construct
GSSGGSGGSGG

SEQ ID NO: 99 - protein, 12 aa, synthetic construct
GSSGGSGGSGGS

SEQ ID NO: 100 - protein, 13 aa, synthetic construct
GSSGGSGGSGGSG

SEQ ID NO: 101 - protein, 16 aa, synthetic construct
GSSGGSGGSGGSGGGS

SEQ ID NO: 102 - protein, 11 aa, synthetic construct
GSSGGSGGSGS

SEQ ID NO: 103 - protein, 5 aa, synthetic construct
GSSGT

SEQ ID NO: 104 - protein, 5 aa, synthetic construct
GSSSG

SEQ ID NO: 105 - protein, 18 aa, synthetic construct
GSTSGSGKPGSGEGSTKG

SEQ ID NO: 106 - protein, 19 aa, synthetic construct
GTAAAGAGAAGGAAAGAAG

SEQ ID NO: 107 - protein, 19 aa, synthetic construct
GTSGSSGSGSGGSGSGGGG

SEQ ID NO: 108 - protein, 14 aa, synthetic construct
IRPRAIGGSKPRVA

SEQ ID NO: 109 - protein, 18 aa, synthetic construct
KESGSVSSEQLAQFRSLD

SEQ ID NO: 110 - protein, 15 aa, synthetic construct
KTTPKLEEGEFSEAR

SEQ ID NO: 111 - protein, 19 aa, synthetic construct
PRGASKSGSASQTGSAPGS

SEQ ID NO: 112 - protein, 6 aa, synthetic construct
QPKAAP

SEQ ID NO: 113 - protein, 13 aa, synthetic construct
QPKAAPSVTLFPP

SEQ ID NO: 114 - protein, 27 aa, synthetic construct
RADAAAAGGGGSGGGGSGGGGSGGGGS

SEQ ID NO: 115 - protein, 12 aa, synthetic construct
RADAAAAGGPGS

SEQ ID NO: 116 - protein, 6 aa, synthetic construct
RADAAP

SEQ ID NO: 117 - protein, 9 aa, synthetic construct
RADAAPTVS

SEQ ID NO: 118 - protein, 6 aa, synthetic construct
SAKTTP

SEQ ID NO: 119 - protein, 18 aa, synthetic construct
SAKTTPKLEEGEFSEARV

SEQ ID NO: 120 - protein, 10 aa, synthetic construct
SAKTTPKLGG

SEQ ID NO: 121 - protein, 14 aa, synthetic construct
STAGDTHLGGEDFD

SEQ ID NO: 122 - protein, 5 aa, synthetic construct
TVAAP

SEQ ID NO: 123 - protein, 12 aa, synthetic construct
TVAAPSVFIFPP

SEQ ID NO: 124 - protein, 24 aa, synthetic construct
TVAAPSVFIFPPTVAAPSVFIFPP

SEQ ID NO: 125 - protein, 400 aa, synthetic construct
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 400 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 127 - protein, 400 aa, synthetic construct
HFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 129 - protein, 395 aa, synthetic construct
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 533 aa, synthetic construct
KSCIHRSQIHVVEGEPFYLKPCGISAPVHRNETATMRWFKGSASHEYRELNNRSSPRVTFHDHTLEFWPVEMEDEGTYISQVGNDRRNWTLNVTKRNKHSCFSDKLVTSRDVEVNKSLHITCKNPNYEELIQDTWLYKNCKEISKTPRILKDAEFGDEGYYSCVFSVHHNGTRYNITKTVNITVIEGRSKVTPAILGPKCEKVGVELGKDVELNCSASLNKDDLFYWSIRKEDSSDPNVQEDRKETTTWISEGKLHASKILRFQKITENYLNVLYNCTVANEEAIDTKSFVLVRKGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 131 - protein, 395 aa, synthetic construct
NFGRLHCTTAVIRNINDQVLFVDKRQPVFEDMTDIDQSASEPQTRLIIYMYKDSEVRGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 710 aa, synthetic construct
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQSGGGGSGGGGSGGGGSKSCIHRSQIHVVEGEPFYLKPCGISAPVHRNETATMRWFKGSASHEYRELNNRSSPRVTFHDHTLEFWPVEMEDEGTYISQVGNDRRNWTLNVTKRNKHSCFSDKLVTSRDVEVNKSLHITCKNPNYEELIQDTWLYKNCKEISKTPRILKDAEFGDEGYYSCVFSVHHNGTRYNITKTVNITVIEGRSKVTPAILGPKCEKVGVELGKDVELNCSASLNKDDLFYWSIRKEDSSDPNVQEDRKETTTWISEGKLHASKILRFQKITENYLNVLYNCTVANEEAIDTKSFVLVRKGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 133 - protein, 221 aa, synthetic construct
CPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 134 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 235 aa, synthetic construct
DKKVEPKSCDKTHTCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK